(19) United States
(12) Patent Application Publication
(10) Pub. No.: US 2013/0225433 A1
(43) Pub. Date: Aug. 29, 2013

(54) PROSTATE CANCER MARKERS AND USES THEREOF

(71) Applicants: Arul M. Chinnaiyan, Plymouth, MI (US); Scott A. Tomlins, Ann Arbor, MI (US)

(72) Inventors: Arul M. Chinnaiyan, Plymouth, MI (US); Scott A. Tomlins, Ann Arbor, MI (US)

(73) Assignee: THE REGENTS OF THE UNIVERSITYS OF MICHAGAN, Ann Arbor, MI (US)

(21) Appl. No.: 13/780,585

(22) Filed: Feb. 28, 2013

Related U.S. Application Data
(60) Provisional application No. 61/604,955, filed on Feb. 29, 2012.

(51) Int. Cl. C12Q 1/68; G01N 33/68

(52) U.S. Cl. CPC C12Q 1/6886; G01N 33/6893
USPC 506/9; 435/7.1

(57) ABSTRACT

The present invention relates to compositions and methods for cancer diagnosis, research and therapy, including but not limited to, cancer markers. In particular, the present invention relates to mutations in cancer markers as diagnostic markers and clinical targets for prostate cancer.

This application claims priority to U.S. Provisional Application No. 61/604,955, filed Feb. 29, 2012, which is herein incorporated by reference in its entirety.

### STATEMENT REGARDING FEDERALLY SPONSORED RESEARCH OR DEVELOPMENT

This invention was made with government support under CA111275, CA113913 and CA69568 awarded by the National Institutes of Health and W81XWH-09-2-0014 awarded by the Army Medical Research and Material Command. The government has certain rights in the invention.

### FIELD OF THE INVENTION

The present invention relates to compositions and methods for cancer diagnosis, research and therapy, including but not limited to, cancer markers. In particular, the present invention relates to mutations in cancer markers as diagnostic markers and clinical targets for prostate cancer.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed.

### SUMMARY OF THE INVENTION

The present invention relates to compositions and methods for cancer diagnosis, research and therapy, including but not limited to, cancer markers. In particular, the present invention relates to mutations in cancer markers as diagnostic markers and clinical targets for prostate cancer.

Embodiments of the present invention provide compositions, kits, and methods useful in the detection and screening of prostate cancer. For example, in some embodiments, the present invention provides a method of screening for or diagnosing metastatic castrate resistant prostate cancer (CRPC) in a sample from a subject, comprising: (a) contacting a biological sample from a subject with a reagent for detecting a mutation in one or more cancer marker genes (e.g., including but not limited to, v-ets erythroblastosis virus E26 oncogene homolog 2 (avian) (ETS2), Myeloid/lymphoid or mixed-lineage leukemia (MLL), Myeloid/lymphoid or mixed-lineage leukemia 3 (MLL3), Myeloid/lymphoid or mixed-lineage leukemia 5 (MLL5), Myeloid/lymphoid or mixed-lineage leukemia 2 (MLL2), Forkhead box A1 (FOXA1), Lysine (K)-specific demethylase 6A (UTX), or ASXL1); and (b) detecting the presence of a mutation in one more of the cancer marker genes using an in vitro assay, wherein the presence of the mutation is indicative of CRCP in the subject. In some embodiments, the sample is tissue, blood, plasma, serum, urine, urine supernatant, urine cell pellet, semen, prostatic secretions or prostate cells. In some embodiments, detection is carried out utilizing a method selected from, for example, a sequencing technique, a nucleic acid hybridization technique, a nucleic acid amplification technique, or an immunoassay. In some embodiments, the nucleic acid amplification technique is, for example, polymerase chain reaction, reverse transcription polymerase chain reaction, transcription-mediated amplification, ligase chain reaction, strand displacement amplification, or nucleic acid sequence based amplification. In some embodiments, the reagent is of a pair of amplification oligonucleotides and an oligonucleotide probe. In some embodiments, the mutation is a loss of function mutation. In some embodiments, the ETS2 mutation is R437c, the MLL mutation is Q1815fp, the MLL3 mutation is R1742fs or F4463fs, the MLL5 mutation is E1397fs, the ASXL2 mutation is Y1163*, Q1104*, Q172*, P749fs, L2240V or R2248*, and the FOXA1 mutation is S453fs or F400I.

In further embodiments, the present invention provides a method of screening for the presence of metastatic castrate resistant prostate cancer (CRPC) in a sample from a subject, comprising: (a) contacting a biological sample from a subject with a reagent for detecting a deletion of ETS2; and (b) detecting the presence of a deletion of ETS2 using an in vitro assay, wherein the present of the deletion is indicative of CRCP in the subject.

The present invention additionally provides a method of screening for the presence of prostate cancer in a sample from a subject, comprising (a) contacting a biological sample from a subject with a reagent that specifically detects a deletion of SPOPL; and (b) detecting the presence of a deletion of SPOPL using an in vitro assay, wherein the presence of the deletion is indicative of prostate cancer in the subject.

Additional embodiments are described herein.

### DESCRIPTION OF THE FIGURES

FIG. 1 shows integrated mutational landscape of lethal metastatic castrate resistant prostate cancer (CRPC). a. Genome wide copy number analysis of each sample was performed using exome sequencing. b. Heatmap of high-level copy number alterations and non-synonymous mutations.

FIG. 2 shows that integrated exome sequencing and copy number analysis highlights novel aspects of ETS genes in prostate cancer biology. a. Genome wide copy number analysis of castrate resistant prostate cancer and high-grade localized prostate cancer was performed using exome sequencing. b. As in a, except from a prostate cancer copy number profiling study by Taylor et al. (Cancer Cell 18, 11-22 (2010)) using array CGH (aCGH). c. Co-expression of CHD1 and ETS family members was analyzed using Oncomine. d. Co-occurrence of CHD1 deregulation (CHD1−) and ETS gene fusions (ETS+) from the current exome study and three prostate cancer copy number profiling studies (aCGH) in Oncomine (Exome/aCGH), 9 prostate cancer gene expression profiling studies in Oncomine (Gene Expr.), and both sets of studies (All). e-g. ETS2 is a prostate cancer tumor suppressor deregulated through deletion and mutation. e. As in a, but centered on the peak of copy number loss on chr 21 between TMPRSS2 and ERG (consistent with TMPRSS2:ERG fusions through deletion). f. Domain structure of ETS2, with the Pointed (gray) and ETS DNA binding domains (black) indicated. g. VCaP prostate cancer cells (ERG+) stably expressing wild type (wt) ETS2 (black), ETS2 R437c (yellow) or LACZ as control (purple) were generated and evaluated for cell migration (left panel), invasion (middle panel) and proliferation (right panel).

FIG. 3 shows that castrate resistant prostate cancer (CRPC) harbors mutational aberrations in chromatin/histone modifiers that physically interact with AR. a. Interaction of deregulated chromatin/histone modifiers with AR. b. As in a, but reverse immunoprecipitation with the indicated chromatin/histone modifier and western blotting for AR. c. VCaP cells were treated with siRNAs against MLL or ASH2L (or non-targeting as control), starved, stimulated with vehicle or 1 nm R1881 for the indicated times and harvested. d. Summary of genes interacting with AR that are deregulated in CRPC.

FIG. 4 shows that recurrent mutations in the androgen receptor (AR) collaborating factor FOXA1 promote tumor growth and disrupt AR signaling. a. Exome sequencing and subsequent screening of 147 localized (n=101) and CRPCs (n=46) identified 5 samples with FOXAl mutations, and sequencing of 11 prostate cancer cell lines identified indels in LAPC-4 and DU-145.

b. Wild type FOXA1 (wt, black) and FOXA1 mutants observed in clinical samples were cloned and expressed in LNCaP cells as Nterminal FLAG fusions (empty vector, purple, used as control) through lentiviral infection. c. Cell proliferation in 1% charcoal-dextran stripped serum with 10 nM DHT was measured by WST-1 colorimetric assay (absorbance at 450 nM) at the indicated time points. Mean+S.E. (n=4) are plotted; * indicates p<0.05 from two tailed t-test. d. FOXA1 wild-type and mutations identified in prostate cancer repress androgen signaling. e.
Representative photographs and quantification of colonies formed in the absence (starved, white) or presence of 1 nm R1881 are shown. Mean+S.E. (n=3) are plotted; * indicates p values<0.05 from two tailed t-test. f. As in e, but subcutaneous xenografts using the indicated cells. Tumor volume is plotted at the indicated time points and representative tumors are shown. For e and f, mean±S.E. (n=3) are plotted; * indicates p values<0.05 from two tailed t-test.

FIG. 5 shows somatic mutation validation as a function of the number of reads calling the variant and the total number of reads.

FIG. 6 shows tumor content estimates across prostate cancer samples.

FIG. 7 shows mutational burden of castrate resistant metastatic prostate cancer (CRPC).

FIG. 8 shows deletion of genes involved in DNA repair in hypermutated CRPC samples.

FIG. 9 shows mutation spectrum of prostate cancer. The percentage of coding somatic mutations for each of the six classes of base substitutions and indels are shown for a) both castrate resistant prostate cancer (CPRC) and localized prostate cancer (PC), b) just CRPC, and c) just PC.

FIG. 10 shows somatic mutations in three different metastatic foci from the same patient confirm the monoclonal origin of lethal metastatic castrate resistant prostate cancer. Venn diagram displaying somatic mutations, including missense, nonsense, indels, and splice site, identified in the celiac lymph node metastatic site (WA43-27), the lung metastatic site (WA43-71), and the bladder local extension/metastatic site (WA43-44).

FIG. 11 shows genome wide copy number analysis by exome sequencing and identification of 1 copy and >1 copy gains/losses. a. Distribution histogram of all Log 2 copy number ratios (tumor to normal) for each targeted exon in WA15. b. Genome wide copy number aberrations for WA15.

FIG. 12 shows comparison of copy number aberrations identified by exome sequencing in castrate resistant prostate cancer (CRPC) and localized prostate cancer. For all genes, the sum of somatic copy number calls (+/−1: one copy gain or loss, respectively; +/−2: high level copy gain/loss, respectively) across a) all profiled samples, b) only CRPC samples or c) only localized prostate cancers was plotted and ordered by genome location (WA43-24 and -71 are excluded from a and b).

FIG. 13 shows a comparison of copy number profiling studies of prostate cancer. a. aCGH profiling of localized prostate cancer (PC, n=59) and CRPC (n=35) was uploaded into Oncomine for analysis and visualization. b. As in a, except the overall sum of log 2 copy numbers from three individual prostate cancer profiling studies available in Oncomine (Demichelis et al. 48, n=49, localized PC; Taylor et al. 16, n=218, localized and hormone treated localized PC and metastatic PC; and TCGA, n=64, localized PC) are plotted.

FIG. 14 shows differential expression of DLX1 between benign prostate tissue and localized prostate cancer. a. Gene expression profiles from benign prostate tissues (n=28), localized prostate cancer (PC, n=59), and CRPC (n=35, not shown), including samples subjected to exome sequencing, were loaded into Oncomine for automated analysis. b. DLX1 expression was measured by qPCR in 10 benign prostate tissues (all included in gene expression profiling), 55 localized PCs (samples included or not included in gene expression profiling indicated in cyan and dark blue, respectively) and 7 metastatic CRPCs (samples included or not included in gene expression profiling indicated in black and gray, respectively). c. Expression of DLX1 by western blotting in 4 benign prostate tissues, 7 localized prostate cancers and 8 metastatic CRPCs. (3-actin was used as loading control.

FIG. 15 shows significantly mutated PTEN protein-interaction subnetwork. a. Matrix indicating the mutations observed in each sample and gene in the PTEN subnetwork, according to the legend. b. Network graph showing the interactions (edges) between proteins (nodes) and indicating the percentage of samples with mutations affecting each protein, classified by type: indel, amplification (AMP), copy number loss (DEL), missense, nonsense and splice site.

FIG. 16 shows identification of high level, focal copy number aberrations in prostate cancer. a. Genome wide copy number analysis of each sample was performed using exome sequencing. b. As in a, but only the sum of high level copy gains/losses (+/−2) is plotted. c. Table showing genes with maximum of high level copy number aberrations.

FIG. 17 shows deregulation of genes at 5q21, including CHD1, confirmed by matched aCGH and gene expression profiling. a. Genome wide analysis by aCGH identified a similar peak of copy number loss on 5q21 (upper panel, sum log 2 copy number across all samples plotted) centered on CHD1. b. Co-expression of CHD1 and ETS family members. c. Genome wide copy number plot for T65, which shows focal, high level deletion of 5q21, including PJA2, but not CHD1. d. Expression of PJA2 stratified by benign prostate tissues, localized prostate cancers and CRPCs (black).

FIG. 18 shows CHD1 deregulation deletion in ETS fusion negative prostate cancer. Prostate cancer copy number profiling studies (by aCGH) from a) The Cancer Genome Atlas (TCGA) and b) Demichelis et al. were accessed at Oncomine.

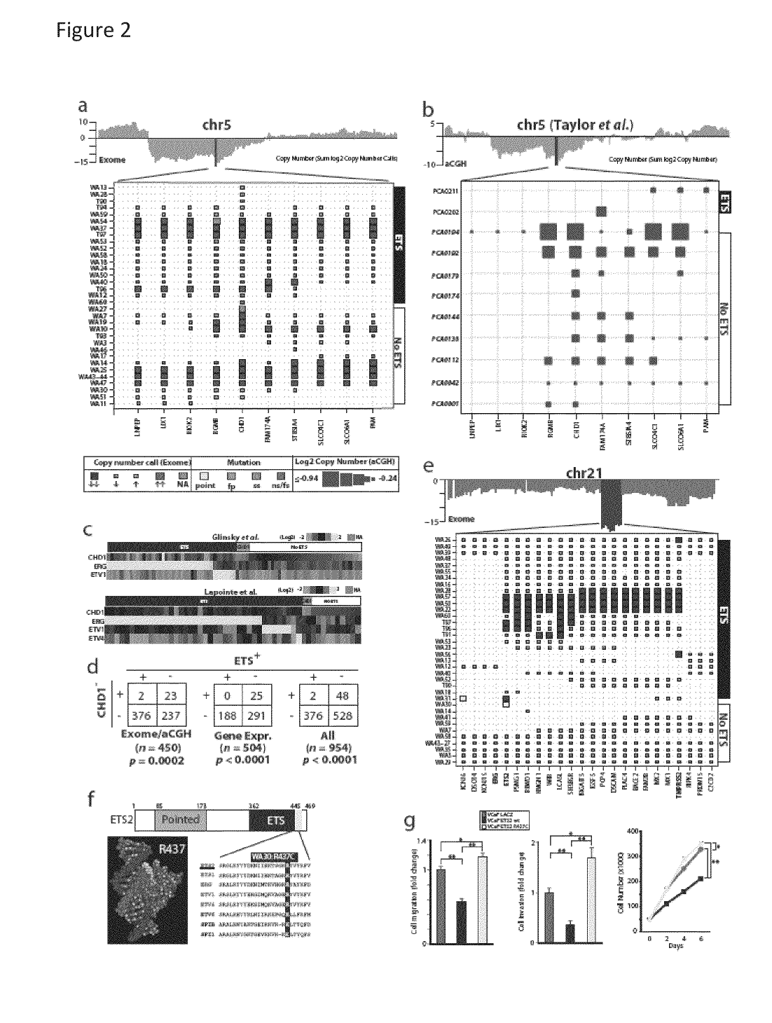

FIG. 19 shows ETS2 expression in prostate tissue samples and cell lines utilized for in vitro assays. a. Gene expression profiles from benign prostate tissues (n=28), localized prostate cancer (PC, n=59), and metastatic castrate resistant prostate cancer (CRPC, n=35), including samples subjected to exome sequencing, were loaded into Oncomine for automated analysis. b. VCaP prostate cancer cells (ERG+) stably expressing wild type (wt) ETS2 or ETS2 R437c with N-terminal HA tag, or LACZ as control, were generated using lentiviruses (see FIG. 2).

FIG. 20 shows confirmation of interaction between ASH2L and androgen receptor (AR), and siRNA knockdown of ASH2L and MLL. a. Reverse immunoprecipitation using two anti-ASH2L antibodies, an antibody against MLL, or IgG control, with western blotting for androgen receptor (AR). 1% whole lysate was used as control. b. VCaP cells were treated with siRNAs against ASH2L or MLL (or non-targeting as control).

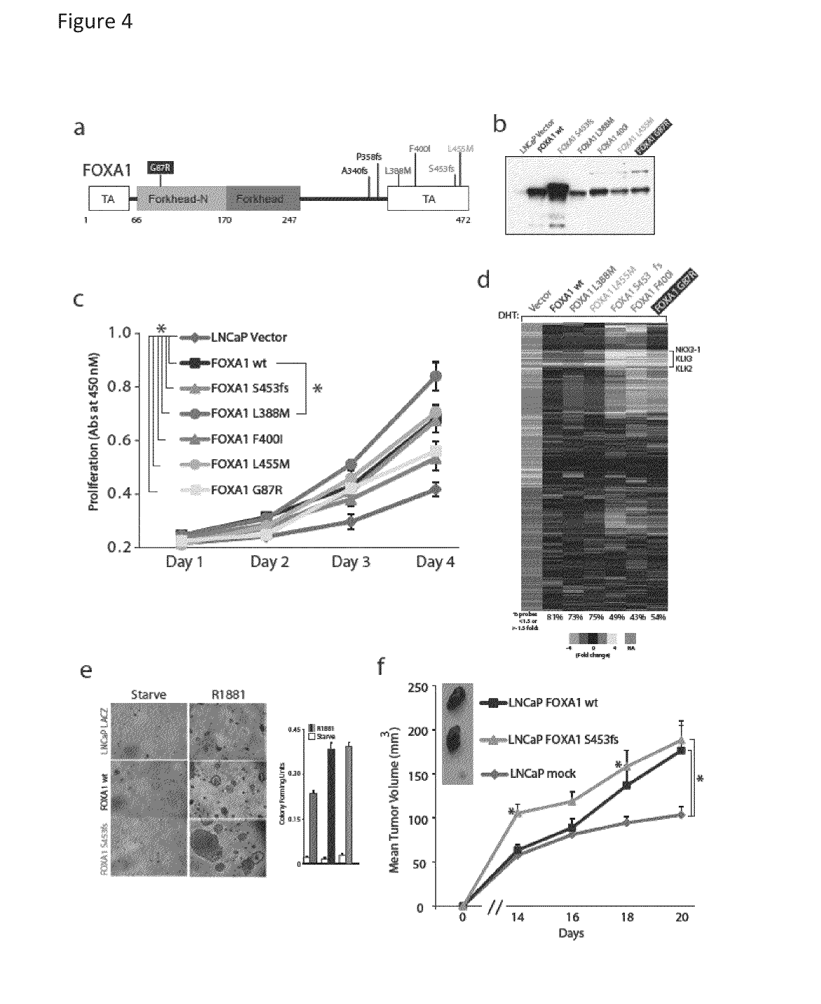

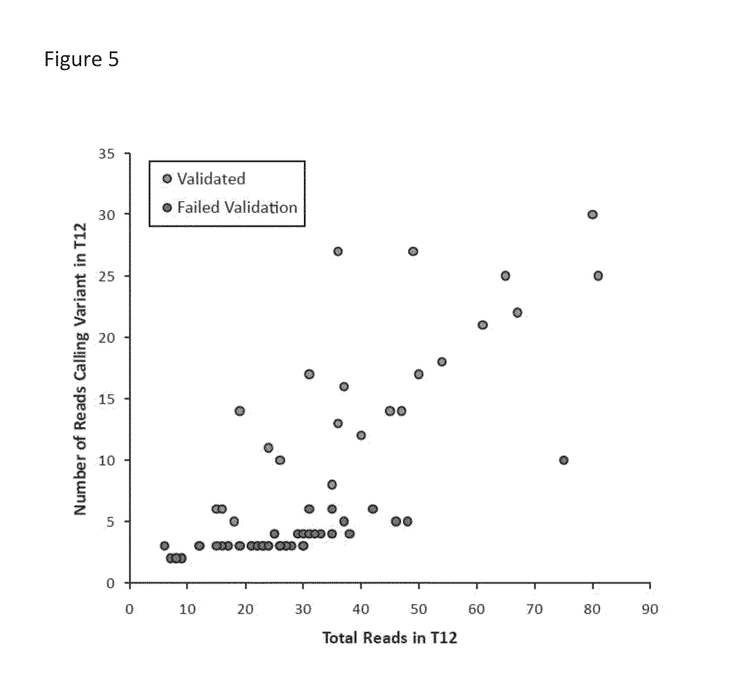

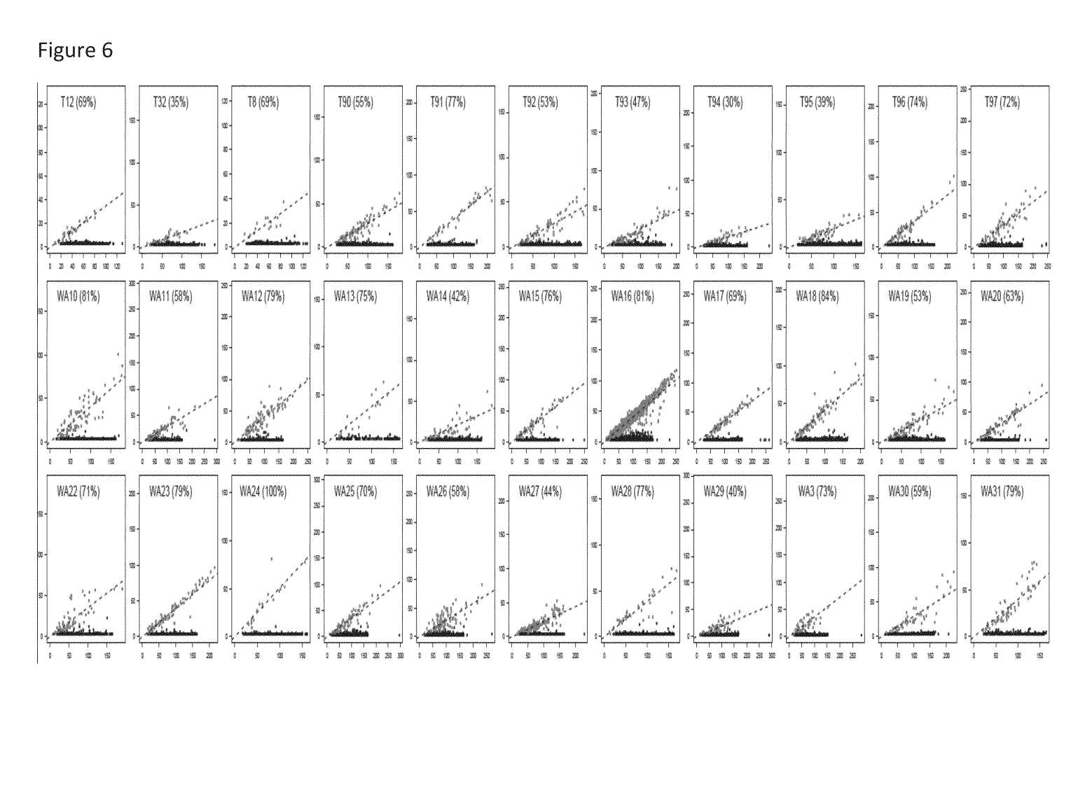

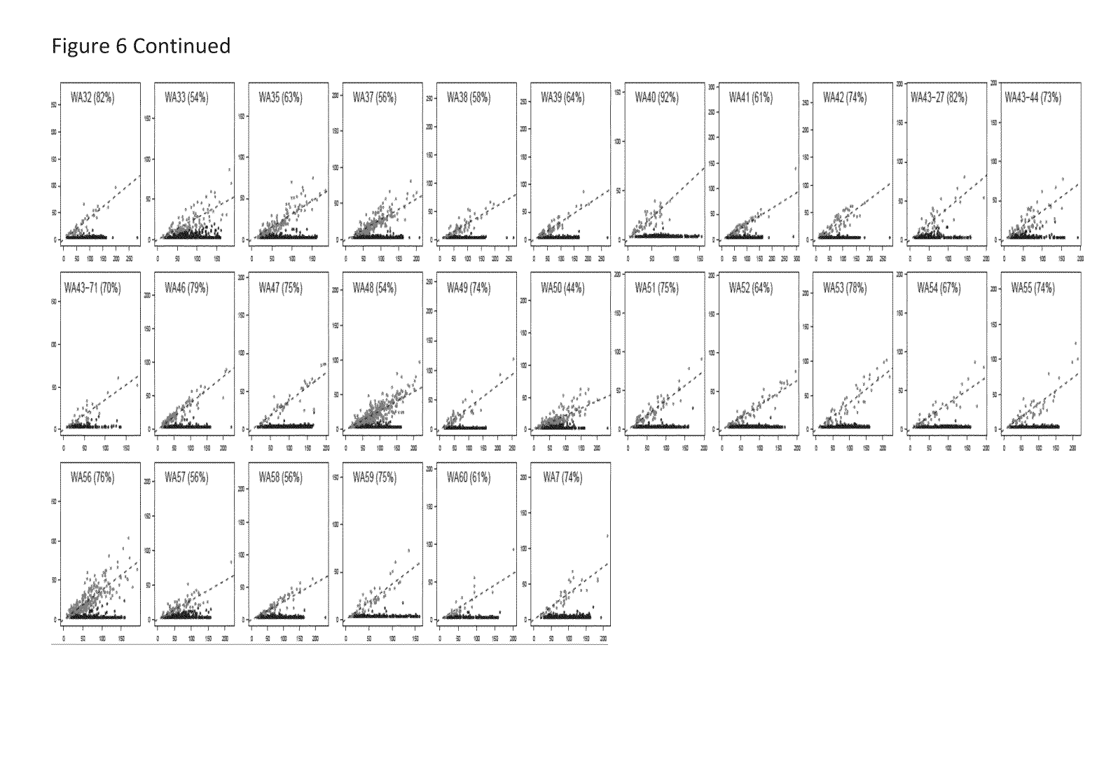

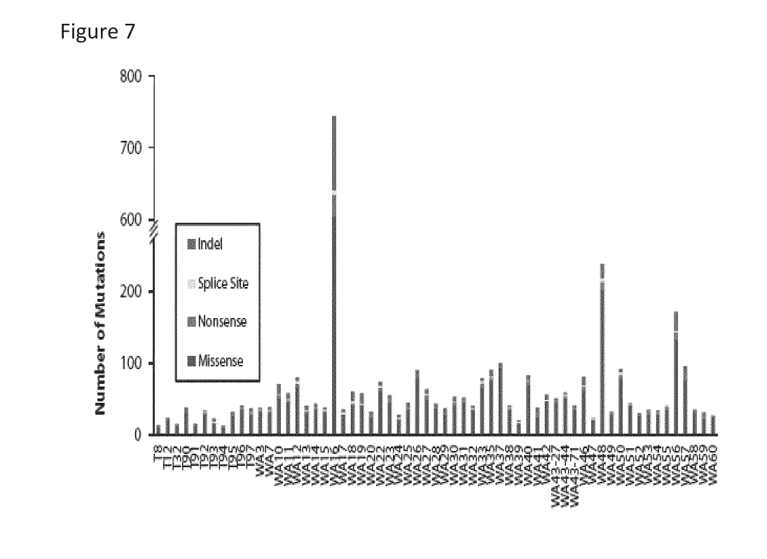

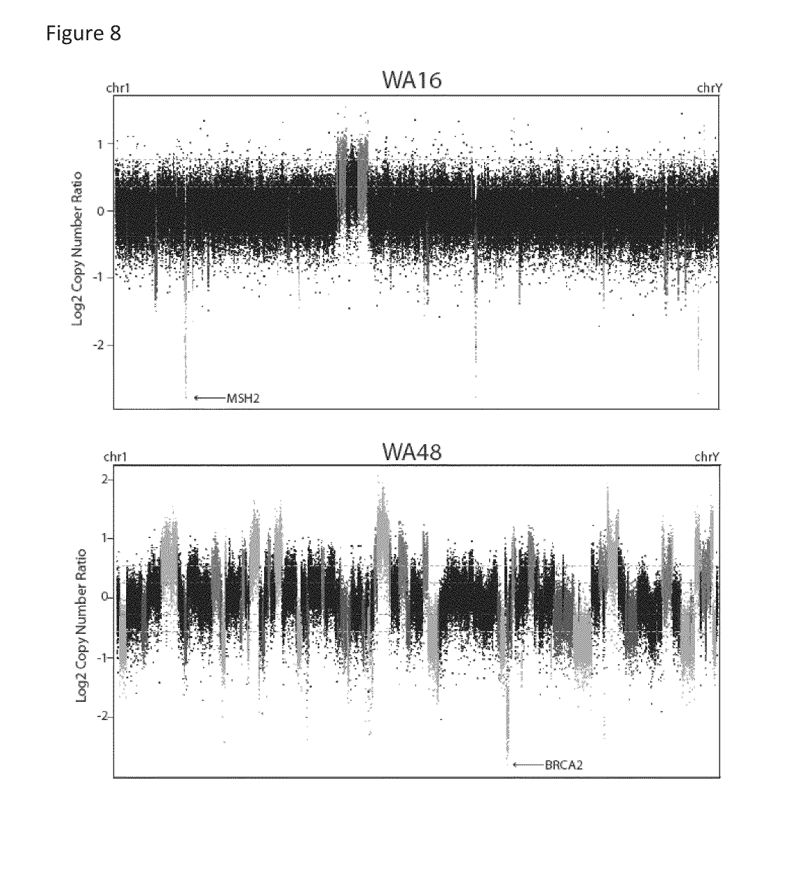

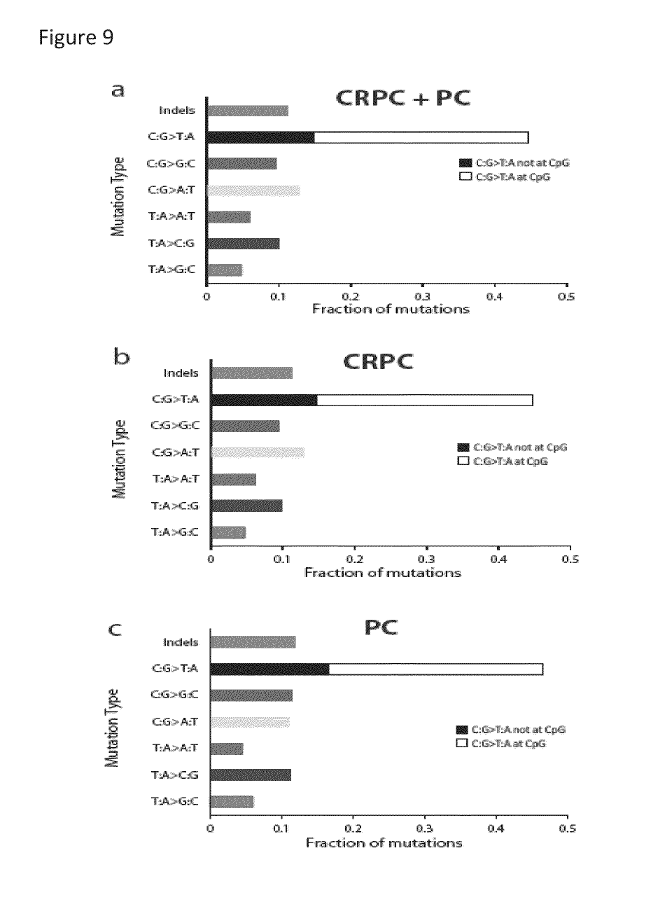

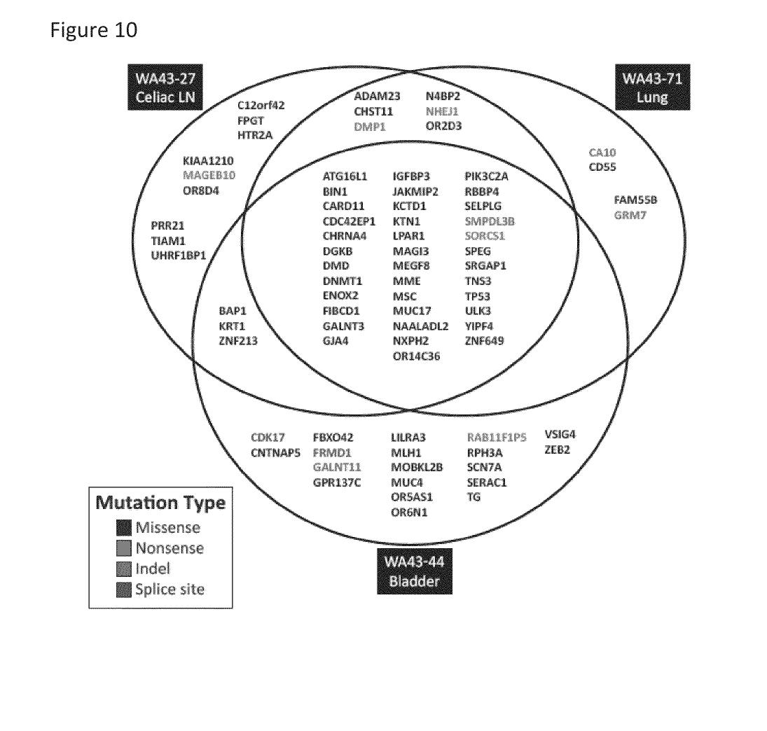

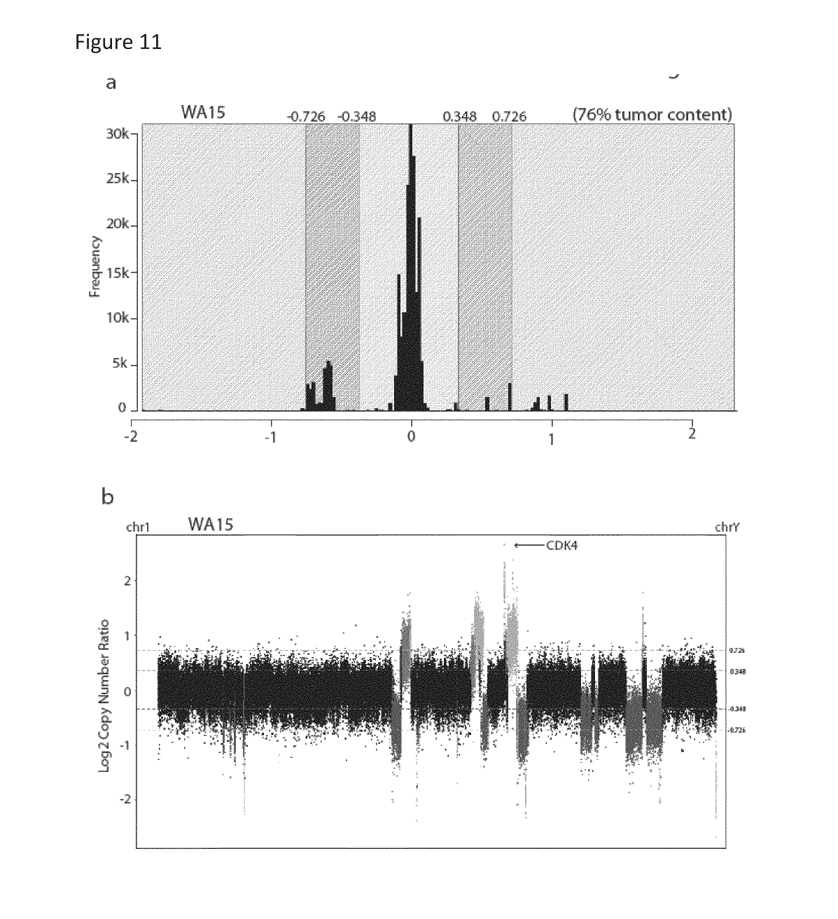

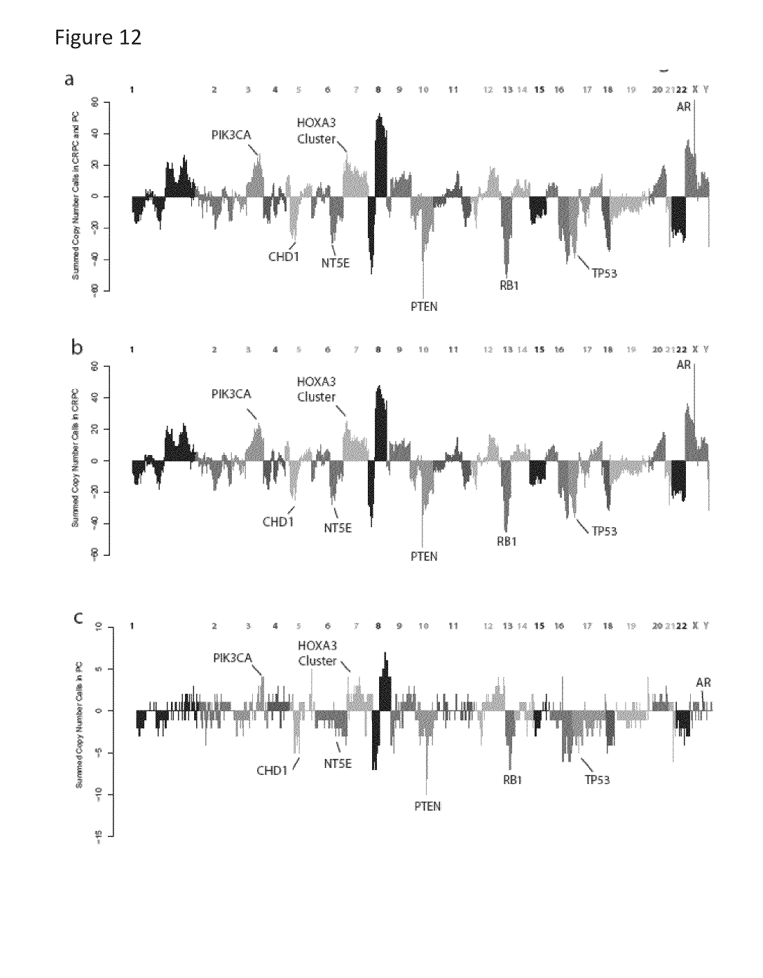

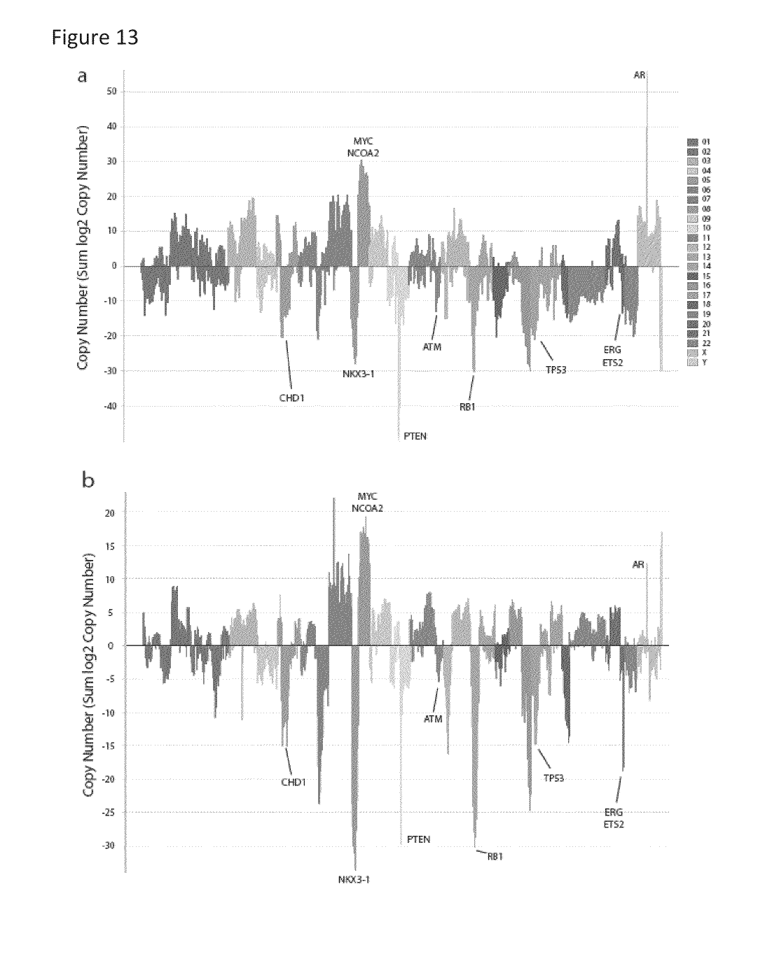

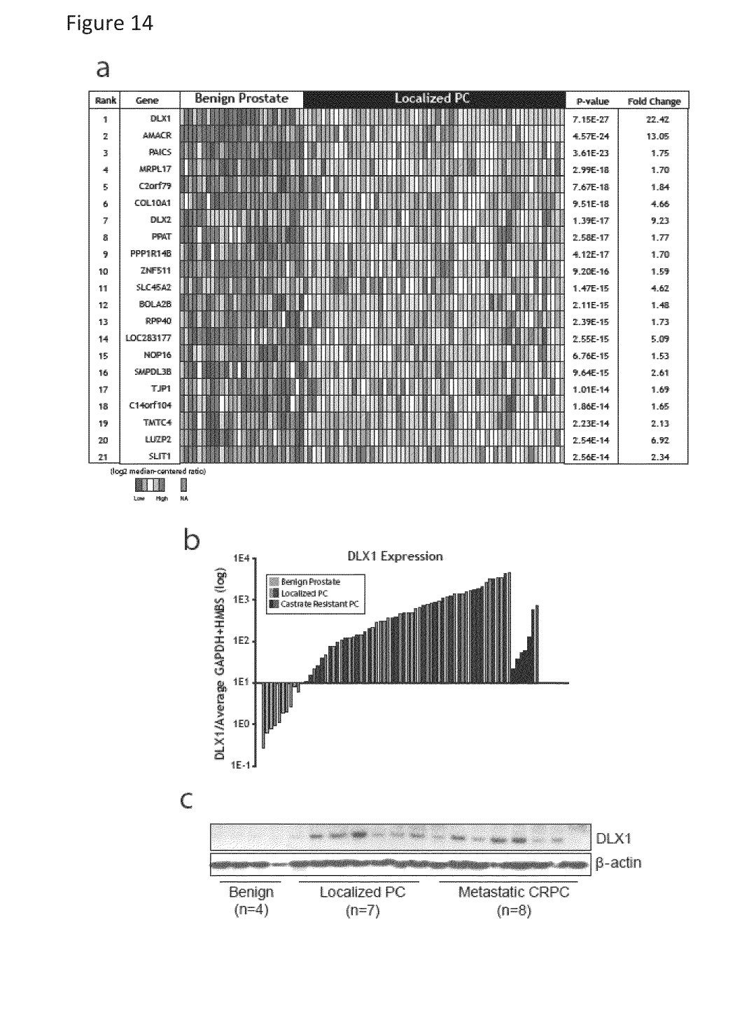

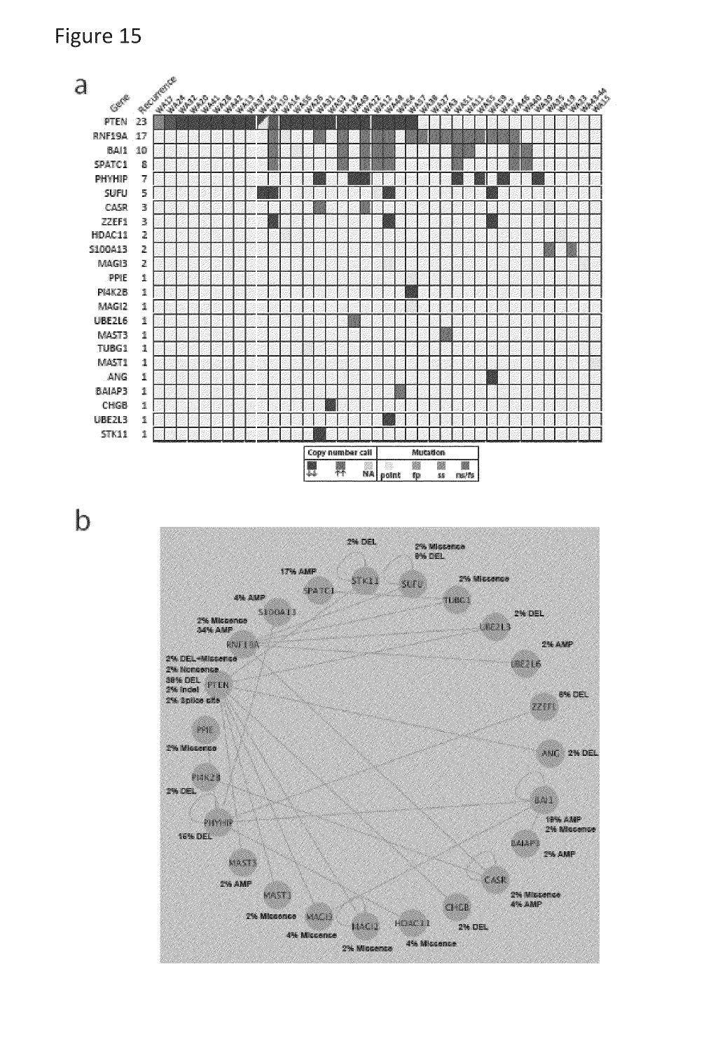

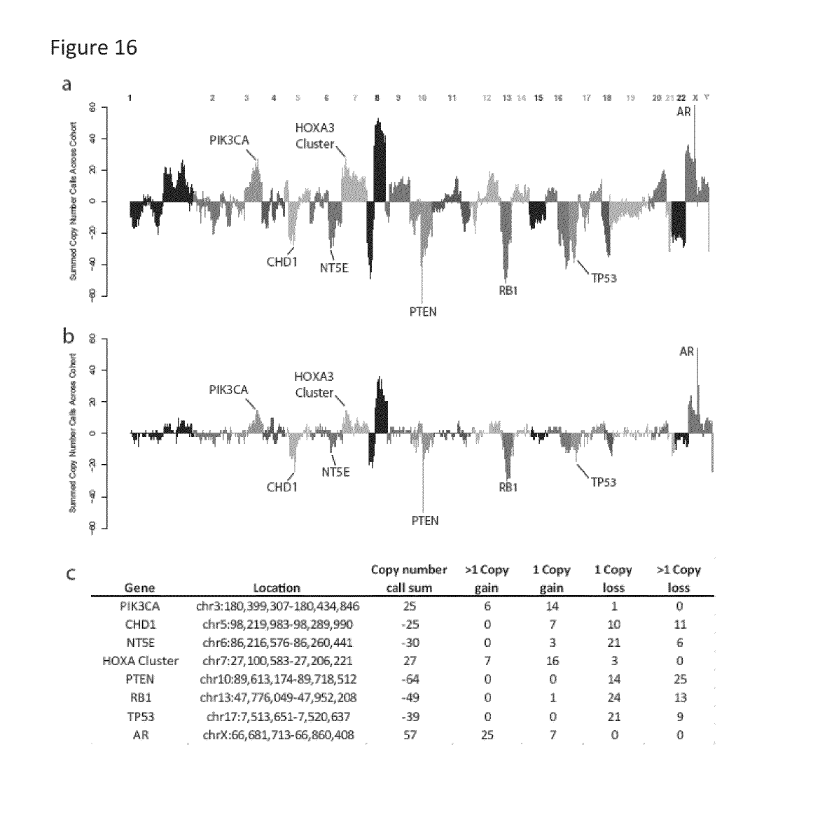

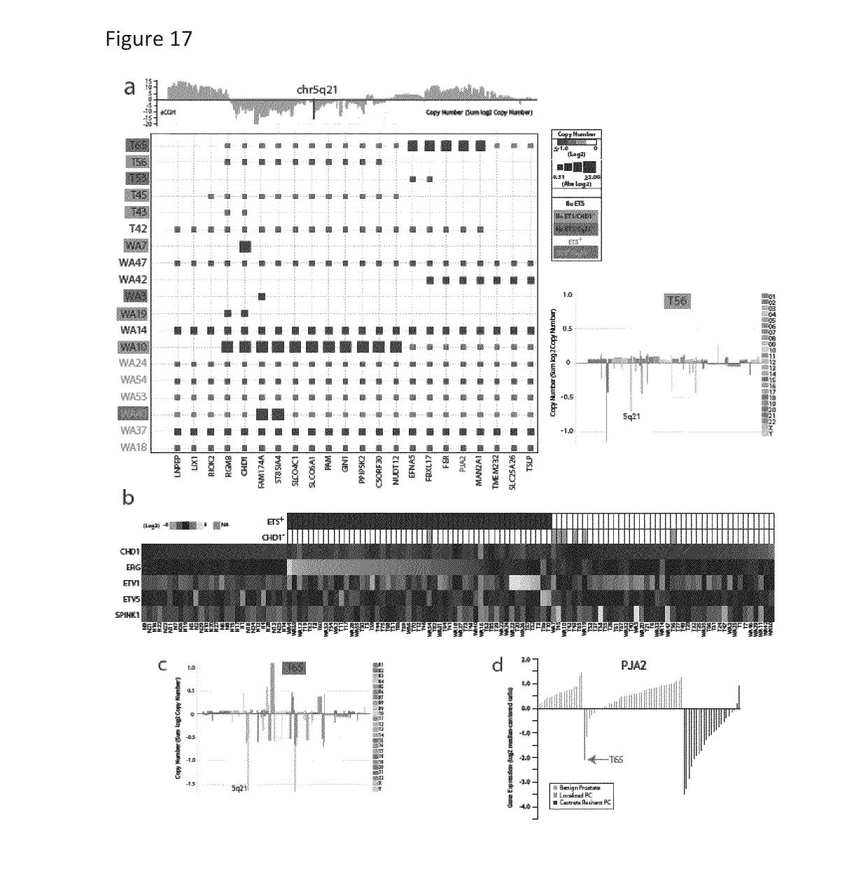

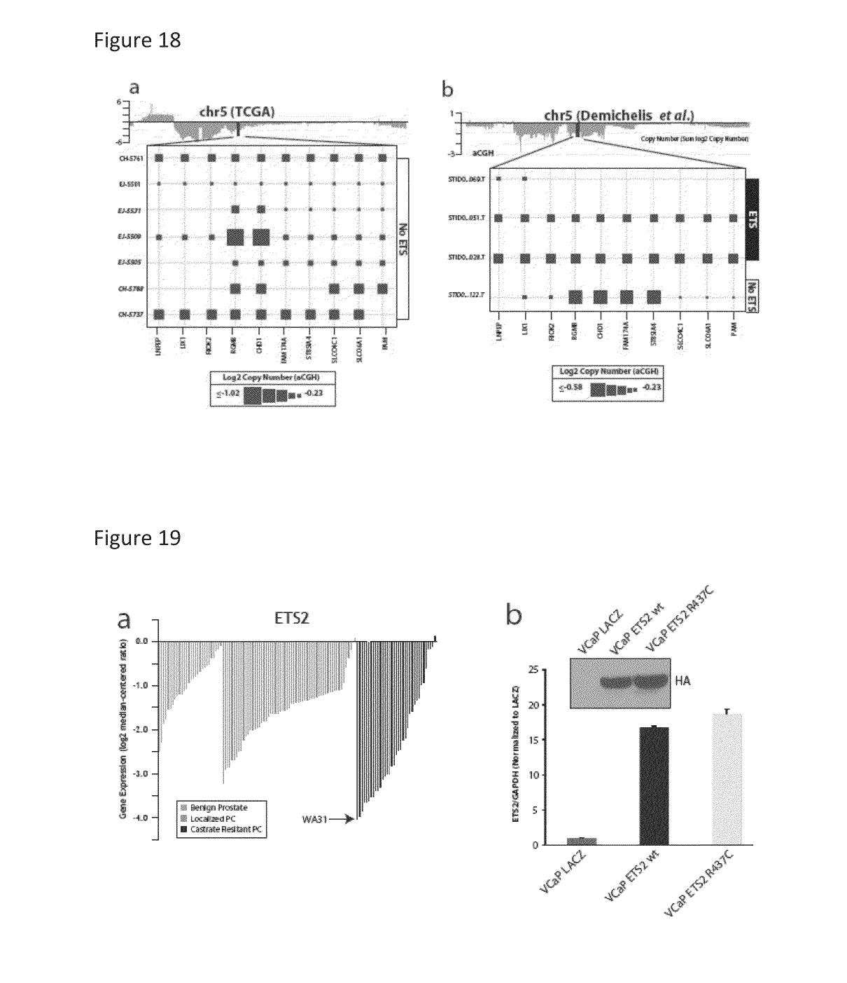

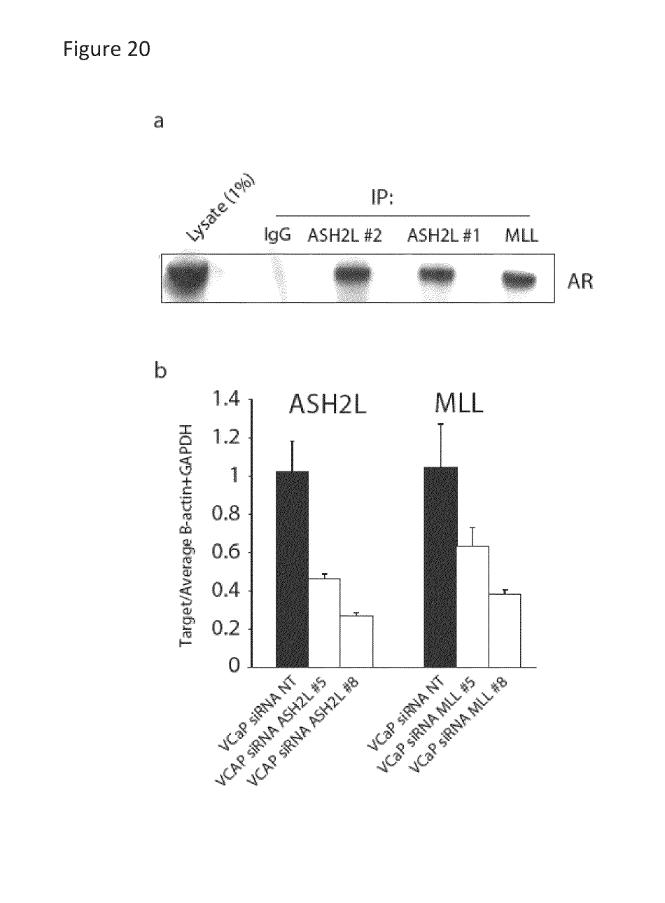

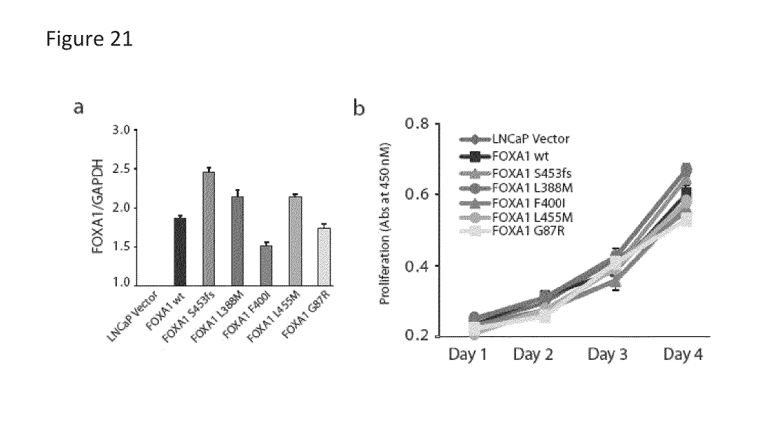

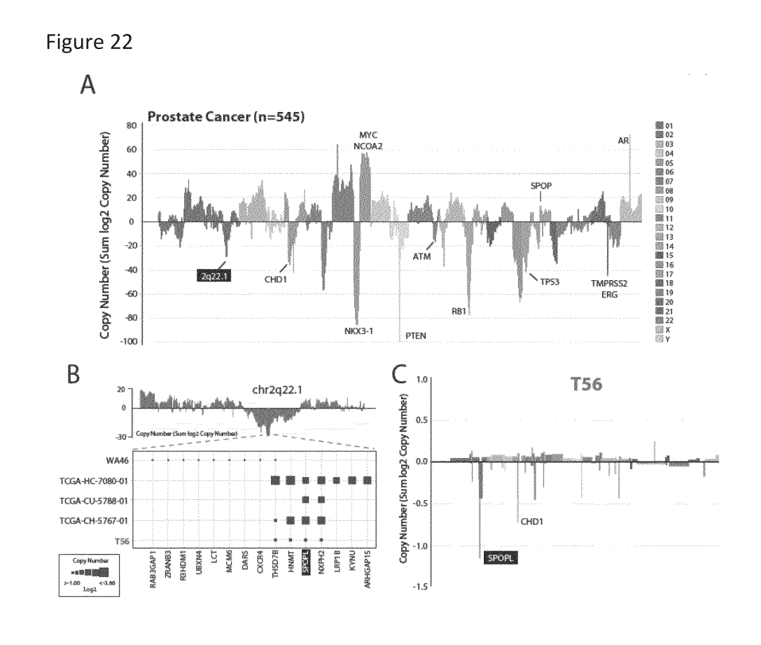

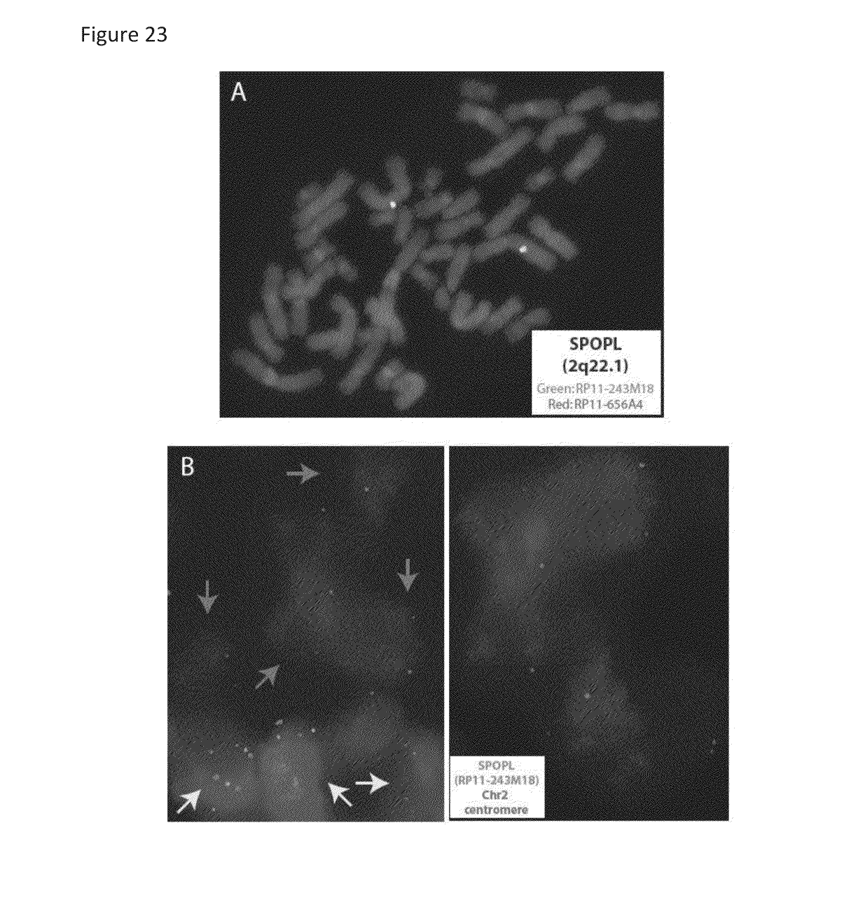

FIG. 21 shows expression of FOXA1 mutants and proliferation in the absence of androgen. a. Wild type FOXA1 (wt, black) and FOXA1 mutants observed in clinical samples were cloned and expressed in LNCaP cells as N-terminal FLAG fusions (empty vector, used as control) through lentiviral infection (see FIG. 4). b. Cell proliferation in 1% charcoal-dextran stripped serum was measured by WST-1 colorimetric assay (absorbance at 450 nM) at the indicated time points.

Mean+S.E. (n=3) are plotted.

FIG. 22 shows that copy number profiling identifies focal deletion of SPOPL in prostate cancer. A. Genome wide copy number profiles from 545 prostate cancers from 4 studies were visualized using the Oncomine Powertools DNA Copy Number Browser. B. High resolution view of chromosome 2 from A. C. Genome wide copy number plot for T56.

FIG. 23 shows fluorescence in situ hybridization (FISH) confirms homozygous deletion of SPOPL in T56. A. FISH probes were generated from BAC clones overlying SPOPL on 2q22.1 (RP11-243M18; RP11-656A4). B. Probes for SPOPL (RP11-243M18) and chromosome 2 centromeric region (Abbot Molecular) were applied to formalin fixed paraffin embedded tissue sections from T56, a localized prostate cancer with homozygous SPOPL deletion by aCGH.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or presence or absence or mutation status in cancer markers described herein indicative of cancer is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the term “characterizing prostate tissue in a subject” refers to the identification of one or more properties of a prostate tissue sample (e.g., including but not limited to, the presence of cancerous tissue, the presence or absence or mutation status of cancer markers, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for cancer diagnosis, research and therapy, including but not limited to, cancer markers. In particular, the present invention relates to mutations in cancer markers as diagnostic markers and clinical targets for prostate cancer.

### I. Diagnostic and Screening Methods

In some embodiments, the present invention provides compositions and method for screening for or diagnosing metastatic castrate resistant prostate cancer (CRPC), distinguishing CRPC from localized prostate cancer, or identifying cancers that are likely to progress from localized prostate cancer to CRPC. For example, experiments conducted during the course of developments of embodiments of the present invention identified mutations in one or more of ETS2, MLL, MLL2, FOXA1, UTX, and ASXL1 and/or deletion of ETS2 in CRPC. Accordingly, in some embodiments, the present invention provides methods of identifying CRPC or localized prostate cancer likely to progress to CRPC based on mutations in one or more cancer markers (e.g., including but not limited to, ETS2, MLL, MLL2, FOXA1, UTX, or ASXL1).

v-ets erythroblastosis virus E26 oncogene homolog 2 (avian) (ETS2) has accession number NM_{—}005239. In some embodiments, ETS2 is deleted or has a R437c mutation in CRPC.

Myeloid/lymphoid or mixed-lineage leukemia (MLL) genes (e.g., MLL, MLL2; accession number NM_{—}003482, MLL3 and MLL5) also demonstrated mutations in CRPC. In some embodiments, Q1815fp mutation in MLL, R1742fs and F4463fs in MLL3, and E1397fs in MLL5 are associated with CRPC.

Additional sex combs like 2 (Drosophila) (ASXL2) has accession number NM_{—}018263 and exhibits Y1163*, Q1104*, Q172*, P749fs, L2240V and R2248* mutations in CRCP.

Lysine (K)-specific demethylase 6A (UTX or KDM6A) has accession number NM_{—}021140 exhibits copy number alterations in CRCP.

Forkhead box A1 (FOXA1) has accession number NM_{—}004496 and exhibits S453fs and F400I mutations in CRCP and/or localized PCA.

In some embodiments, assays identify recurrent deletions in ETS2 and/or SPOPL.

speckle-type POZ protein-like (SPOPL) has the accession number NM_{—}001001664 and is deleted in prostate cancer.

Any patient sample suspected of containing the cancer markers may be tested according to methods of embodiments of the present invention. By way of non-limiting examples, the sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

In some embodiments, the patient sample is subjected to preliminary processing designed to isolate or enrich the sample for the cancer markers or cells that contain the cancer markers. A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

The cancer markers may be detected along with other markers in a multiplex or panel format. Markers are selected for their predictive value alone or in combination with the gene fusions. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); RAS/KRAS (Bos, Cancer Res. 49:4682-89 (1989); Kranenburg, Biochimica et Biophysica Acta 1756:81-82 (2005)); and, those disclosed in U.S. Pat. Nos. 5,854,206 and 6,034,218, 7,229,774, each of which is herein incorporated by reference in its entirety. Markers for other cancers, diseases, infections, and metabolic conditions are also contemplated for inclusion in a multiplex or panel format.

i. DNA and RNA Detection

Mutations in the cancer markers of the present invention are detected using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength. A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing (see, e.g., Astier et al., J. Am. Chem. Soc. 2006 Feb. 8; 128(5):1705-10, herein incorporated by reference) is utilized. The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the HeliScope by Helicos BioSciences technology is utilized (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

In some embodiments, the nucleic acid sequencing approach developed by Stratos Genomics, Inc. and involves the use of Xpandomers is utilized. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

2. Hybridization

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts (e.g., cancer markers) within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, cancer markers or loss of cancer markers are detected using fluorescence in situ hybridization (FISH). In some embodiments, FISH assays utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present invention further provides a method of performing a FISH assay on human prostate cells, human prostate tissue or on the fluid surrounding said human prostate cells or human prostate tissue. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

3. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., cancer markers or mutated cancer markers) by comparing gene expression or mutation status in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Nucleic acids (e.g., cancer markers) may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified nucleic acids can be detected by any conventional means.

For example, the cancer markers can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

In some embodiments, nucleic acids are detected and characterized by the identification of a unique base composition signature (BCS) using mass spectrometry (e.g., Abbott PLEX-ID system, Abbot Ibis Biosciences, Abbott Park, Ill.,) described in U.S. Pat. Nos. 7,108,974, 8,017,743, and 8,017,322; each of which is herein incorporated by reference in its entirety.

ii. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data.

Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a cancer marker) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

iiii. In vivo Imaging

Cancer markers may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of embodiments of the present invention are useful in the identification of cancers that exhibit mutated or deleted cancer markers described herein (e.g., prostate cancer). In vivo imaging is used to visualize the presence or level of expression of a cancer marker. Such techniques allow for diagnosis without the use of an unpleasant biopsy. The in vivo imaging methods of embodiments of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the cancer marker, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

iv. Compositions & Kits

Compositions for use in the diagnostic methods described herein include, but are not limited to, probes, amplification oligonucleotides, and the like.

The probe and antibody compositions of the present invention may also be provided in the form of an array.

### II. Drug Screening Applications

In some embodiments, the present invention provides drug screening assays (e.g., to screen for anticancer drugs). The screening methods of the present invention utilize cancer markers described herein. For example, in some embodiments, the present invention provides methods of screening for compounds that alter (e.g., increase or decrease) the expression or activity of cancer markers described herein. The compounds or agents may interfere with transcription, by interacting, for example, with the promoter region. The compounds or agents may interfere with mRNA (e.g., by RNA interference, antisense technologies, etc.). The compounds or agents may interfere with pathways that are upstream or downstream of the biological activity of cancer markers. In some embodiments, candidate compounds are antisense or interfering RNA agents (e.g., oligonucleotides) directed against cancer markers. In other embodiments, candidate compounds are antibodies or small molecules that specifically bind to a cancer markers regulator or expression products and inhibit its biological function.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of cancer markers is assayed for by detecting the level of cancer marker expressed by the cell. mRNA expression can be detected by any suitable method.

### EXPERIMENTAL

The following examples are provided in order to demonstrate and further illustrate certain preferred embodiments and aspects of the present invention and are not to be construed as limiting the scope thereof.

### Example 1

### A. Methods

### Tissue Samples and Cell Lines

Prostate tissues were from the radical prostatectomy series at the University of Michigan and from the Rapid Autopsy Program (Rubin, M. A. et al. Clin Cancer Res 6, 1038-1045 (2000)), both of which are part of the University of Michigan Prostate Cancer Specialized Program of Research Excellence (SPORE) Tissue Core. All samples were collected with informed consent of the patients and previous institutional review board approval.

The immortalized prostate cancer cell lines 22Rv1, C4-2B, CWR22, DU-145, LAPC-4, LNCaP, MDA-PCa-2B, NCI-H660, PC3, VCaP and WPE1-NB26 (Table 5) were obtained from the American Type Culture Collection (Manassas, Va.). PC3, DU-145, LNCaP, 22Rv1, and CRW22 cells were grown in RPMI 1640 (Invitrogen) and supplemented with 10% fetal bovine serum (FBS) and 1% penicillin-streptomycin. VCaP cells were grown in DMEM (Invitrogen) and supplemented with 10% fetal bovine serum (FBS) with 1% penicillinstreptomycin. NCl-H660 cells were grown in RPMI 1640 supplemented with 0.005 mg/ml insulin, 0.01 mg/ml transferrin, 30 nM sodium selenite, 10 nM hydrocortisone, 10 nM betaestradiol, 5% FBS and an extra 2 mM of L-glutamine (for a final concentration of 4 mM). MDAPCa-2B cells were grown in F-12K medium (Invitrogen) supplemented with 20% FBS, 25 ng/ml cholera toxin, 10 ng/ml EGF, 0.005 mM phosphoethanolamine, 100 pg/ml hydrocortisone, 45 nM selenious acid, and 0.005 mg/ml insulin. LAPC-4 cells were grown in Iscove's media (Invitrogen) supplemented with 10% FBS and 1 nM R1881. C4-2B cells were grown in 80% DMEM supplemented with 20% F12, 5% FBS, 3 g/L NaCo3, 5 ug/ml insulin, 13.6 pg/ml triiodothyonine, 5 ug/ml transferrin, 0.25 ug/ml biotin, and 25 μg/ml adenine. WPE1-NB26 cells were grown in Keratinocyte Serum Free Medium (Invitrogen) and supplemented with bovine pituitary extract (BPE, 0.05 mg/ml) and human recombinant epidermal growth factor (EGF, 5 ng/ml). Androgen treated LNCaP and VCaP cell line samples were also generated for transcriptome analysis, using cells grown in androgen-depleted media lacking phenol red and supplemented with 10% charcoal-stripped serum and 1% penicillin-streptomycin. After 48 hours, cells were treated with 5 nM methyltrienolone (R1881, NEN Life Science Products) or an equivalent volume of ethanol. Cells were harvested for RNA isolation at 6, 24, and 48 hours post-treatment.

High Molecular Weight Genomic DNA (gDNA) Isolation

Frozen tissue samples were taken as chunks or sections from OCT-embedded, flash frozen tissue blocks. gDNA was isolated using the Qiagen DNeasy Blood & Tissue Kit according to the manufacturer's instructions. Briefly, cell or tissue lysates were incubated at 56° C. in the presence of proteinase K and SDS, purified on silica membrane-based mini-columns, and eluted in buffer AE (10 mM Tris-HCl, 0.5 mM EDTA pH 9.0).

### Generation of Exome-Capture Libraries

Exome libraries of matched pairs of tumor/normal genomic DNAs (Table 1) were generated using the Illumina Paired-End Genomic DNA Sample Prep Kit, following the manufacturers' instructions. 3 μg of each genomic DNA was sheared using a Covaris S2 to a peak target size of 250 bp. Fragmented DNA was concentrated using AMPure XP beads (Beckman Coulter), and DNA ends were repaired using T4 DNA polymerase, Klenow polymerase, and T4 polynucleotide kinase. 3′ A-tailing with exo-minus Klenow polymerase was followed by ligation of Illumina paired-end adapters to the genomic DNA fragments. The adapter-ligated libraries were electrophoresed on 3% Nusieve 3:1 (Lonza) agarose gels and fragments between 300 to 350 bp were recovered using QIAEX II gel extraction reagents (Qiagen). Recovered DNA was then amplified using Illumina PE1.0 and PE2.0 primers for 9 cycles. The amplified libraries were purified using AMPure XP beads and the DNA concentration was determined using a Nanodrop spectrophotometer. 1 mg of the libraries were hybridized to the Agilent biotinylated SureSelect Capture Library at 65° C. for 72 hr or to the Roche EZ Exome capture library at 47° C. for 72 hr following the manufacturer's protocol. The targeted exon fragments were captured on Dynal M-280 streptavidin beads (Invitrogen), washed, eluted, and enriched by amplification with the Illumina PE1.0/PE2.0 primers for 8 additional cycles. After purification of the PCR products with AMPure XP beads, the quality and quantity of the resulting exome libraries were analyzed using an Agilent Bioanalyzer.

### Somatic Point Mutation Identification by Exome Capture Sequencing

All captured DNA libraries were sequenced with the Illumina GAII Genome Analyzer or the Illumina HiSeq in paired end mode, yielding 80 base pairs from the final library fragments. The reads that passed the chastity filter of Illumina BaseCall software were used for subsequent analysis. Next, matepairs were pooled and then mapped as single reads to the reference human genome (NCBI build 36.1, hg18), excluding unordered sequence and alternate haplotypes, using Bowtie (Langmead, et al., Genome Biol 10, R25 (2009)), keeping unique best hits, and allowing up to two mismatched bases per read. Reads in the tumor that mapped to another location in the genome with three mismatches were excluded from further consideration. Likely PCR duplicates were removed by removing reads that have the same match interval on the genomic sequence. Individual basecalls with Phred quality less than Q20 were excluded from further consideration. A mismatched base (SNV) was identified as a somatic mutation only when 1) it had six reads of support (this cut-off was selected based on Sanger validation rates in T12 2) it was in at least 10% of the coverage at that position in the tumor, 3) it was observed on both strands, 4) there was 8× coverage in the matched normal, and 5) it did not occur in the matched normal sample in more than two reads and 2% of the coverage (to ensure that somatic variants are not filtered out due to tumor contamination in the normal, variants present in 2-4% of the coverage in the matched normal were retained if they were in at least 20% of the coverage in the tumor). SNVs were excluded from further consideration as somatic mutations if 1) they did not fall within 50 bases of a target region, 2) they occurred in any two matched normal samples in at least two reads and 2% of the coverage, or 3) they occurred in another tumor and its matched normal sample in two reads and 4% of the coverage.

### Identification of Coding Indels in Exome Capture Data

The methodology for identifying indels in exome capture data was adapted from 24 with minor modifications. Reads for which Bowtie was unsuccessful in identifying an ungapped alignment were converted to fasta format and mapped to the target regions, padded by 200 bases on either side, with cross_match (v0.990329), using parameters -gap_ext-1-bandwidth 10-minmatch 20-maxmatch 24. Output options were -tags -discrep_lists - alignments. Alignments with an indel were then filtered for those that: 1) had a score at least 40 more than the next best alignment, 2) mapped at least 75 bases of the read, and 3) had two or fewer substitutions in addition to the indel. Reads from filtered alignments that mapped to the negative strand were then reverse-complemented and, together with the rest of the filtered reads, remapped with cross_match using the same parameters (to reduce ambiguity in called indel positions due to different read orientations). After the second mapping, alignments were refiltered using criteria 1-3. Reads that had redundant start sites were removed as likely PCR duplicates, after which the number of reads mapping to either the reference or the non-reference allele was counted for each. An indel was called if there were at least six non-reference allele reads making up at least 10% of all reads at that genomic position. Indels were reported with respect to genomic coordinates. For insertions, the position reported is the last base before the insertion. For deletions, the position reported is the first deleted base. Indel somatic mutation candidates were excluded from further consideration if 1) they did not occur on both strands, 2) they did not fall within 50 bases of a target region, 3) there wasn't 8× coverage in the matched normal at that position, 4) they occurred in the matched normal sample in more than 2 reads and 4% of the coverage, 5) they occurred in any two matched normal samples, or 6) they occurred in any single matched normal sample in more than 2 reads.

### Annotation

The resulting somatic mutations were annotated using CCDS transcripts wherever possible.

If no CCDS transcript was available, the coding regions of RefSeq transcripts were used. HUGO gene names were used. The impact of coding nonsynonymous amino acid substitutions on the structure and function of a protein was assessed using PolyPhen-225. It was also assessed whether the somatic variant was previously reported in dbSNP or COSMIC v5626.

### Calculation of Somatic Mutation Rates

The somatic mutation rate was calculated as described (Berger, M. F. et al. Nature 470, 214-220 (2011)). A base was identified as “covered”, if there was at least 14× total coverage after PCR duplicate removal in the tumor and 8× total coverage after PCR duplicate removal in the matched normal sample. Only mutations called at covered annotated targeted positions were covered; the total number of covered annotated targeted positions ranged from 22.3-30.4 Mb per sample, with 74.4-94.3% of annotated targeted positions covered per sample. Because this calculation does not take into consideration the sensitivity of the somatic mutation calling method or tumor purity, it may underestimate the actual mutation rate for the sample.

### Tumor Content Estimation

Tumor content was estimated for each cancer sample by fitting a binomial mixture model with two components to the set of most likely SNV candidates on 2-copy genomic regions. The set of candidates used for estimation consisted of coding variants that (1) exhibited at least >=3 variant fragments in the cancer sample, (2) exhibited zero variant fragments in the matched benign sample with at least 16 fragments of coverage, (3) were not present in dbSNP, (4) were within a targeted exon or within 100 base pairs of a targeted exon, (5) were not in homopolymer runs of four or more bases, and (6) exhibited no evidence of amplification or deletion. In order to filter out regions of possible amplification or deletion, exon coverage ratios were used to infer copy number changes, following the approach of 27. Resulting SNV candidates were not used for estimation of tumor content if the segmented log-ratio exceeded 0.25 in absolute value. Candidates on the X and Y chromosomes were also eliminated because they were unlikely to exist in 2-copy genomic regions.

Using this set of candidates, a binomial mixture model was fit with two components using the R package flexmix, version 2.2-828. One component consisted of SNV candidates with very low variant fractions, presumably resulting from recurrent sequencing errors and other artifacts The other component, consisting of the likely set of true SNVs, was informative of tumor content in the cancer sample. Specifically, under the assumption that most or all of the observed SNV candidates in this component are heterozygous SNVs, we expect the estimated binomial proportion of this component to represent one-half of the proportion of tumor cells in the sample. Thus, the estimated binomial proportion as obtained from the mixture model was doubled to obtain an estimate of tumor content in each sample.

### Determination of Significantly Mutated Genes and Pathways

The determination of significantly mutated genes and pathways was done as described (Berger et al., supra; Chapman, M. A. et al. Nature 471, 467-472 (2011).) using methodology based on that of Getz et al. (Science 317, 1500 (2007)) and Ding et al. (Nature 455, 1069-1075 (2008)). Before doing the calculations, one of the three samples derived from distinct metastatic sites from the same individual (WA43) was selected for inclusion in the sample set in order to ensure that the requirement of independence was met for the set of considered mutations. WA43-44 was selected because it contained all of the recurrent somatic mutations that occurred in WA43-27 or WA43-71, along with additional recurrent mutations not contained in the other two. Hyper-mutated sample WA16 was excluded. In this approach, significantly mutated genes are identified based on the observed number of mutations for each sequence context-based mutation class (CpG, other C:G, A:T, and indels), the sample-specific and class-specific background mutation rates, and the number of covered bases per gene. Before calculating the background mutation rate, genes that have been reported in the literature as having recurrent somatic mutations in prostate cancer: AR, TP53, CHEK2, KLF6, EPHB2, ZFHX3, NCOA2, PLXNB1, SPTA1, and SPOP were excluded (Berger et al. supra; Taylor, B. S. et al. Cancer Cell 18, 11-22 (2010); Yamaoka, et al., Clin Cancer Res 16, 4319-4324 (2010); Huusko, P. et al. Nat Genet. 36, 979-983 (2004); Sun, X. et al. Nat Genet. 37, 407-412 (2005); Dong, J. T. J Cell Biochem 97, 433-447 (2006); Agell, L. et al. Mod Pathol 21, 1470-1478 (2008); Narla, G. et al. Science 294, 2563-2566 (2001); Wong, O. G. et al. Proc Natl Acad Sci USA 104, 19040-19045 (2007).). The resulting background mutation rate for localized prostate cancer samples was 5.03/MB for CpG, 0.71/Mb for other C:G, 0.39/Mb A:T and 0.10/Mb indels. The resulting background mutation rate for metastatic prostate cancer samples was 8.45/MB for CpG, 1.80/Mb for other C:G, 0.95/Mb A:T and 0.21/Mb indels. For each gene, the probability of obtaining the observed set of mutations (or a more extreme one) given the observed background mutation rates was calculated. P-values are converted to q-values using the Benjamini-Hochberg procedure for controlling False Discovery Rate (FDR).

This analysis was repeated to consider significantly mutated pathways, considering a list of 880 gene sets corresponding to the set of canonical pathways used in Gene Set Enrichment Analysis (GSEA). For this analysis, the number of mutations and the number of covered bases in all component genes of each gene set and the total number of covered bases in the set were tabulated. As in the single-gene analysis, the mutation counts were broken down into the context-based mutation classes (CpG, other C:G, and A:T), and then the P-value and subsequent q-value were calculated.

### Sanger Sequencing to Validate Somatic Point Mutations and Indels

Various genomic locations nominated for somatic point mutations and indels were amplified from whole genome amplified DNA (Kim, J. H. et al. Cancer Res 67, 8229-8239 (2007)) from corresponding matched normal-tumor tissue pairs or cell lines. Briefly, fifty ng of input genomic DNA was subjected to fragmentation, library preparation and amplification steps using Genomeplex-Complete Whole Genome Amplification Kit (Sigma-Aldrich) according to manufacturer's instructions. The final whole genome amplified DNA was purified by AMPure XP beads (Beckman-Coulter) and quantified by a Nanodrop spectrophotometer (Thermo Scientific). Fifty ng of DNA were used as template in PCR amplifications with HotStar Taq DNA polymerase (Qiagen) with the suggested initial denaturation and cycling conditions. Primer sequences were as described (Jones, S. et al. Science 321, 1801-1806 (2008)) and are based on human hg18, March 2006 assembly. Primers for FOXA1 can be found in Table 13. The PCR products were subjected to Sanger sequencing by the University of Michigan DNA Sequencing Core after treatment with ExoSAP-IT (GE Healthcare) and sequences were analyzed using MacVecotr software (MacVector).

### Exome Copy Number Analysis Copy

Copy number aberrations were quantified and reported for each gene as the segmented normalized log 2-transformed exon coverage ratios between each tumor sample and its matched normal as described (Lonigro, R. J. et al. Neoplasia 13, 1019-1025 (2011)). Sample-specific cutoffs, based on estimated tumor content, were used to define regions of gain and loss, as follows. For a sample with tumor percentage P, genomic regions with N copies in cancer cells and 2 copies in normal cells would be predicted to give log-ratios centered at log 2(N*P+2*(1-P))-1. For each sample, using its estimated tumor content the predicted locations of these N-copy peaks in the distribution of log-ratios were computed and cutoffs were chosen to fall between these predicted peaks. To define high-level gains (e.g., greater than 3 copies), the weighted average of the 3-copy and 4-copy predicted peaks was computed with weights 0.25 and 0.75, respectively.

Similarly, to define low-level gains (e.g., greater than 2 copies), the weighted average of the 2-copy and 3-copy predicted peaks was computed, using the same weights. These weighted averages were used as cut-offs to define high-level gain and low-level gain, respectively. Next, the negatives of the cutoffs for high-level gain and low-level gain were used as the cut-offs for high-level loss (two-copy loss) and low-level loss (single-copy loss), respectively. Histograms of the distributions of segmented log 2 copy number ratios were then examined (FIG. 11) and cutoffs adjusted manually in cases in which this algorithmic approach appeared to misclassify large numbers of genomic regions (due to lower tumor content, multiple clones, severe aneuploidy, etc). All cutoffs with estimated tumor percentages are given in Table 14. The resulting copy number alterations were reported for all sixty one prostate cancer tumors with +2 representing high-level (>1 copy) gain, +1 representing 1 copy gain, 0 representing no change, −1 representing 1 copy loss, and −2 representing high-level (>1 copy) loss (Table 20). To identify potential drivers, all called copy number alterations were summed across all samples and identified genes with the maximum number of high level gains or losses occurring in peaks summed copy number gain or loss, respectively. For all analyses and visualizations, WA43-27 and WA43-71 were excluded.

### Identification of Significantly Mutated Protein-Protein Interaction Subnetworks

HotNet (Vandin, et al., J Comput Biol 18, 507-522 (2011)) was used to find subnetworks of a large protein-protein interaction network containing a significant number of mutations and copy number alterations (CNAs). The input to HotNet is a dataset of matched somatic mutations and copy number alterations for a set of tumor samples. The output of HotNet is a list of subnetworks, each containing at least n genes. HotNet employs a two-stage statistical test to assess the significance of the output. In the first stage the p-value for the number of subnetworks in the list is computed. In the second stage the false discovery rate (FDR) of the list of subnetworks is estimated. At the end, the significance of each individual subnetwork in the list is assessed by comparison to known pathways and protein complexes.

Combined somatic mutations and CNAs (generated from exome data [Table 20]), considering only high-level (>1 copy) gains and two-copy losses, were analyzed for the 47 metastatic samples (hyper-mutated sample WA16 was not included and one of the three metastatic sites from the same individual, WA43-44 was included). CNAs for which the sign of aberration was not consistent in at least 90% of the altered samples were removed. The interaction network derived from the Human Protein Reference Database (HPRD) was used (Keshava Prasad, T. S. et al. Human Nucleic Acids Res 37, D767-772 (2009)). For the statistical test, random aberrations were generated as follows. Simulated mutations were observed using the estimated background mutation rate (1.97×10⁻⁶). CNAs from the observed distribution of CNA lengths were simulated, permuting their positions. In this way artifacts resulting from functionally related genes that are both neighbors on the network and close enough on the genome (and thus affected by the same CNA) are minimized. Subnetworks reported by HotNet that contain 3 or more genes in the same CNA in one or more samples were discarded. Moreover, for subnetworks with two genes g1, g2 in the same CNA in 1 or more samples, the genes that were not reported when alterations in either g1 or g2 are removed were removed.

Using the approach above, HotNet identifies 28 candidate subnetworks containing at least genes (p<0.01) with FDR=0.32. A total of 24 subnetworks remained after CNA filtering. Those 24 subnetworks were compared with known pathways in the KEGG database and with protein complexes from PINdb (Mertz et al., Neoplasia 9, 200-206 (2007)). Of the 24 subnetworks, 14 had statistically significant (p<0.05 after Bonferroni correction) overlap with at least one KEGG pathway or protein complex (Table 11).

RNA Isolation and cDNA Synthesis

Total RNA was isolated from frozen prostate tissue samples (for gene expression analysis and qPCR) and cell lines (for transcriptome sequencing, qPCR/expression profiling from cell lines) using Trizol (or Qizol [Qiagen]) and an RNeasy Kit (Invitrogen) with DNase I digestion according to the manufacturer's instructions. RNA integrity was verified on an Agilent Bioanalyzer 2100 (Agilent Technologies). cDNA was synthesized from total RNA using Superscript 111 (Invitrogen) and random primers (Invitrogen).

### Transcriptome Library Preparation and Sequencing

Next generation RNA sequencing was performed on 11 prostate cell lines according to Illumina's protocol using 2 μg of total RNA. RNA integrity was measured using an Agilent 2100 Bioanalyzer, and only samples with a RIN score>7.0 were advanced for library generation. PolyA+ RNA was selected for using Sera-Mag oligo(dT) beads (Thermo Scientific) and fragmented with the Ambion Fragmentation Reagents kit (Ambion, Austin, Tex.). cDNA synthesis, end-repair, A-base addition, and ligation of the Illumina PCR adaptors (single read or paired-end where appropriate) were performed according to Illumina's protocol. Libraries were then size-selected for 250-300 bp cDNA fragments on a 3.5% agarose gel and PCR-amplified using Phusion DNA polymerase (Finnzymes) for 15-18 PCR cycles. PCR products were then purified on a 2% agarose gel and gel-extracted. Library quality was credentialed by assaying each library on an Agilent 2100 Bioanalyzer for product size and concentration. Libraries were sequenced as 36-45mers on an Illumina Genome Analyzer I or Genome Analyzer II flowcell according to Illumina's protocol. All single read samples were sequenced on a Genome Analyzer I, and all paired-end samples were sequenced on a Genome Analyzer II.

### Somatic Point Mutation Identification in Transcriptome Sequence Data

Transcriptome short reads were trimmed to remove the first two bases and as many bases as necessary to ensure the read length was less than 40 bp. Trimmed short read sequences were mapped to the reference human genome (NCBI build 36.1, hg18), excluding unordered sequence and alternate haplotypes, and the 2008 Illumina splice junction set using Bowtie in single read mode keeping unique best hits and allowing up to two mismatched bases. Matepairs from paired end runs were pooled and treated as single reads. Likely PCR duplicates were removed by removing reads that have the same match interval on the genomic sequence or an exon junction. Individual basecalls with Phred quality less than Q20 were excluded from further consideration. A mismatched base (SNV) was identified as a candidate somatic mutation when it had three reads of support and was in at least 10% of the coverage at that position in the tumor. Less stringent criteria were applied for nominating candidate somatic mutations in the transcriptome as compared to the exome capture data, since only variants in the transcriptome recurrent to known somatic mutations were further considered (see below). SNVs were excluded from further consideration as recurrent somatic mutations if 1) they occurred in any two matched normal exomes in at least two reads and 2% of the coverage, or 2) they occurred in another tumor exome and its matched normal exome in two reads and 4% of the coverage.

### Identification of Coding Indels in Transcriptome Data

The methodology for identifying indels in transcriptome data was adapted from Ng, S. B. et al. (Nature 461, 272-276 (2009)). Reads for which Bowtie was unsuccessful in identifying an ungapped alignment were converted to fasta format and mapped to the set of full-length CCDS transcripts, padded by 32 genomic bases on either side, with cross_match (v0.990329), using parameters -gap_ext-1-bandwidth 10-minscore 24-minmatch 16-maxmatch 24. Output options were -tags - discrep_lists -alignments. Alignments with an indel were then filtered for those that: 1) had a score at least 20 more than the next best alignment; and 2) had two or fewer substitutions in addition to the indel. Reads from filtered alignments that mapped to the negative strand were then reverse-complemented and, together with the rest of the filtered reads, remapped with cross_match using the same parameters (to reduce ambiguity in called indel positions due to different read orientations). After the second mapping, alignments were re-filtered using criteria 1) and 2). Reads that had redundant start sites were removed as likely PCR duplicates, after which the number of reads mapping to either the reference or the non-reference allele were counted for each. An indel was called if there were at least four non-reference allele reads making up at least 10% of all reads at that transcript position. Indels were reported with respect to genomic coordinates. For insertions, the position reported is the last base before the insertion. For deletions, the position reported is the first deleted base. Indel somatic mutation candidates were excluded from further consideration if they were present in dbSNP132, or if they occurred in a single read in any two matched normal exome samples or in a single matched normal exome sample with two or more reads. Identified indel variants are given in Table 6.

### Identification of Transcriptome Somatic SNVs Recurrent to Known Somatic Variants

The somatic mutations identified in the exome data in this example (excluding the eight that did not validate by Sanger sequencing) were combined with the confirmed somatic variants in COSMIC v56 to yield a comprehensive somatic mutation dataset. A transcriptome SNV was considered recurrent to a known somatic variant, if it resulted in the same nucleotide change, amino acid change, or if it disrupted the same amino acid. Identified variants recurrent to our exome data are given in Table 7, and those recurrent to somatic variants in COSMIC are given in Table 8.

Array Comparative Genomic Hybridization (aCGH)

aCGH of 28 benign prostate tissues, 59 localized prostate cancers (including 56 not subjected to exome sequencing) and 35 CRPCs (including 4 not subjected to exome sequencing, see Table 4) was performed using gDNA on Agilent's 105K or 244K aCGH microarrays (Human Genome CGH 105K or 244K Oligo Microarray) using Agilent's standard Direct Method protocol and Wash Procedure B. Briefly, 1.5-3 ug of gDNA from prostate specimens (isolated as above) was restriction digested with Alul and RsaI, labeled with Cy-5 (test channel), purified using Microcon YM-30 columns and hybridized with an equal amount of Cy-3 (reference channel) labeled Human Male Genomic DNA (Promega) for 40 hours at 65° C.

Post-hybridization wash was performed with acetonitrile wash and Agilent Stabilization and Drying Solution wash according to the manufacturer's instructions. Scanning was performed on an Agilent scanner Model G2505B (5 micron scan with software v7.0), and data was extracted using Agilent Feature Extraction software v9.5 using protocol CGH-v4_{—}95_Feb07. For data analysis, probes on all arrays were limited to those on the 105K array. Log(2) ratios for each probe were determined as rProcessedSignal/gProcessedSignal. To remove copy number variants, all probes with log(2) values>1 or <−1 in any of the 28 benign prostate samples were excluded. The final dataset (consisting of localized prostate cancer and castrate resistant metastatic samples) was uploaded into a custom instance of Oncomine for automated copy number analysis. In Oncomine, circular binary segmentation was performed on the dataset using the DNACopy package (v1.18) available via the Bioconductor package. Agilent Probe IDs are mapped to segments and reporter values are used to generate segment values (mean of reporters). Resulting segments are mapped to hg18 (NCBI 36.1) RefSeq coordinates (UCSC refGene) as provided by UCSC (UCSC refGene, July 2009, hg18, NCBI 36.1, March 2006) and segment values are assigned to each gene. Copy number profiles were visualized using Oncomine Power Tools.

### Gene Expression Microarray Analysis

Gene expression microarray analysis of the same prostate tissue samples subjected to aCGH (Table 4) was performed using Agilent Whole Human 44k element arrays (1×44k or 4×44k format) as described (Tomlins, S. A. et al. Nature 448, 595-599 (2007)). RNA from indicated prostate samples were labeled with Cy-5 (test channel) and hybridized against Cy-3 (reference channel) labeled pooled benign prostate RNA (Clontech). Arrays were scanned using an Agilent Model G2505B scanner, and data was extracted using Agilent Feature Extraction software. Control probes were removed from all arrays and the LogRatio for all probes, which were used for subsequent analysis, were converted to log(2). Although the 1×44k and 4×44k arrays have the same probes, the 4×44k arrays have 10 replicates of some probes. Thus, to generate a final data set, the median value of replicated probes was used for 4×44k arrays. The final data set (including benign prostate, localized prostate cancer and CRPC) was uploaded into a custom instance of Oncomine for automated analysis. In Oncomine, the dataset was median centered (per array) prior to indicated analyses.

### ETS/RAF/CHD1 Status

ETS/RAF gene fusion status for all samples was assigned based on expression of TMPRSS2:ERG by qPCR (Tomlins, S. A. et al. Science 310, 644-648 (2005).), outlier expression and/or rearrangement of ERG, ETV1, ETV4 or ETV5 by FISH (Mehra, R. et al. Cancer Res 68, 3584-3590 (2008); Tomlins, S. A. et al. Nature 448, 595-599 (2007); Tomlins, S. A. et al. Science 310, 644-648 (2005); Helgeson, B. E. et al. Cancer Res 68, 73-80 (2008)), RAF family member rearrangement by transcriptome sequencing and subsequent qPCR and FISH validation (Palanisamy, N. et al. Nat Med 16, 793-798 (2010)), presence of deletion between TMPRSS2 and ERG by aCGH, or ERG protein expression by immunohistochemistry (Park, K. et al. Neoplasia 12, 590-598 (2010)). CHD1− status was determined by examination of exome copy number profiles (or aCGH profiles) for all samples, and those with focal deletions involving CHD1 (without a larger focal deletion within 10 MB) or nonsynonymous mutations in CHD1 were considered CHD1−. Assessment of ETS status in aCGH profiling studies in Oncomine was performed as follows, and samples in each study with focal deletions (log 2 ratio<−0.23 or −0.24) or high level focal deletions arising in background deletions were considered CHD1−. For the Demichelis et al. study (supra), ETS+ samples were those identified by the authors as harboring TMPRSS2:ERG gene fusions. For the Taylor et al. study 16, samples with specific deletions between TMPRSS2 and ERG, or those with outlier expression in matched gene expression data of ERG, ETV1, ETV4 or ETV5, were considered ETS+. For the TCGA study, samples with specific deletions between TMPRSS2 and ERG were considered ETS+. For evaluation of ETS/CHD1 status from gene expression profiling studies, 9 prostate cancer profiling studies (Glinsky et al., J Clin Invest 113, 913-923 (2004); Lapointe, J. et al. Proc Natl Acad Sci USA 101, 811-816 (2004); LaTulippe, E. et al. Cancer Res 62, 4499-4506 (2002); Liu, P. et al. Cancer Res 66, 4011-4019 (2006); Tamura, K. et al. Cancer Res 67, 5117-5125 (2007); Wallace, T. A. et al. Cancer Res 68, 927-936 (2008); Welsh, J. B. et al. Cancer Res 61, 5974-5978 (2001); Yu, Y. P. et al. J Clin Oncol 22, 2790-2799 (2004)) (and the International Genomics Consortium's expO dataset) were accessed in Oncomine. In each study, samples with outlier over-expression of ERG, ETV1, ETV4 or ETV5 were considered ETS+, samples with CHD1 outlier under-expression were considered CHD1− and samples with outlier over-expression of SPINK1 were considered SPINK1+.

### ETS2

Full length wild type ETS2 with N-terminal HA-tag was PCR amplified and cloned into pCR8/GW/TOPO vector (Invitrogen). ETS2 R437c was generated using the Quick changemutagenesis kit (Stratagene). ETS2 wildtype and R437c were transferred into pLenti-4-V5 DEST vector (Invitrogen). After confirmation of the insert sequence, lentiviruses were generated by the University of Michigan Vector Core. VCaP cells were infected and stably expressing ETS2 wild type, ETS2 R437c mutant and lacZ control were generated by selection with Zeocin (Invitrogen). ETS2 expression was confirmed by qPCR for ETS2 expression and western blotting with anti-HA antibody as above. For proliferation assays, 50,000 cells (n=4) were plated per well in 24-well poly-lysine coated plates, and cells were harvested and counted at the indicated time points by Coulter counter (Beckman Coulter, Fullerton, Calif.). For in vitro migration and invasion, 2.0×10⁵ cells (migration n=8; invasion n=12) were placed in the top chamber with a noncoated membrane or Matrigel coated membrane, respectively (24-well insert; pore size 8 μm; BD Biosciences). In both the assays, cells were plated in medium without serum, and medium supplemented with 10% serum was used as a chemoattractant in the lower chamber. Cells were incubated for 48 hr and cells that did not migrate or invade through the pores were gently removed with a cotton swab. Cells on the lower surface of the membrane were stained with crystal violet and counted.

AR Interaction with Histone/Chromatin Remodelers

VCaP cells were lysed in Triton X-100 lysis buffer (20 mM MOPS, pH 7.0, 2 mM EGTA, 5 mM EDTA, 30 mM sodium fluoride, 60 mM β-glycerophosphate, 20 mM sodium pyrophosphate, 1 mM sodium orthovanadate, 1% Triton X-100, 1 mM DTT, protease inhibitor cocktail (Roche, #14309200)). Cell lysates (0.5-1.0 mg) were then pre-cleaned with protein A/G agarose beads (Santa Cruz, #sc-2003) by incubation for 1 hour with shaking at 4° C. followed by centrifugation at 2000 rpm for 3 minutes. Antibody coupling reactions were performed according to the Dynabeads Antibody Coupling Kit (Invitrogen, Cat#143.11D). Briefly, 10 mg Dynabeads M-270 were washed with buffer and mixed with primary antibody as indicated. Reactions were then incubated on a roller at 37° C. overnight (16-24 hours), washed with buffer and resuspended to a final concentration of 10 mg antibody coupled beads/mL. Lysates were then incubated overnight with the coupled antibodies as indicated. The mixture was then incubated with shaking at 4° C. for another 4 hours or overnight prior to washing the lysate-bead precipitate (centrifugation at 2000 rpm for 3 minutes) 4 times in Triton X-100 lysis buffer. Beads were finally precipitated by centrifugation, resuspended in 25 L of 2× loading buffer and boiled at 80° C. for 10 minutes for separation of proteins and beads.

Samples were then analyzed by SDS-PAGE and transferred onto Polyvinylidene Difluoride membrane (GE Healthcare, Piscataway, N.J.). The membrane was then incubated in blocking buffer [Tris-buffered saline, 0.1% Tween (TBS-T), 5% nonfat dry milk] for 1 hours at room temperature with the following: anti-ASH2L rabbit polyclonal (1:4000 in blocking buffer, Bethyl lab #A300-489A), anti-MLL mouse monoclonal (1:1000 in blocking buffer, Millipore#05-765), anti-AR rabbit polyclonal (1:1000 in blocking buffer, Millipore Cat #06-680), anti-FOXA1 mouse monoclonal (1:2000 in blocking buffer, Abcam Cat #ab23738), anti-UTX mouse monoclonal (1:1000 in blocking buffer, Abcam #ab91231), anti-MLL2-Rabbit polyclonal (1:2000 in blocking buffer, Bethyl lab Cat #A300-113A), anti-ASXL2 Rabbit polyclonal (1:2000 in blocking buffer, Abcam Cat #ab69420), anti-CHD1 (1:4000 in blocking buffer, Bethyl lab Cat#A310-411A) and anti-ERG (1:1000 in blocking buffer, Epitomics Cat #EPR 3864. Following washes with TBS-T, the blot was incubated with horseradish peroxidaseconjugated secondary antibody and the signals visualized by enhanced chemiluminescence system as described by the manufacturer (GE Healthcare).

Knockdown of ASH2L or MLL in VCaP cells was accomplished by RNA interference using commercially available siRNA duplexes for ASH2L (Dharmacon, Cat#J-019831-05 and J-019831-08) and MLL (Dharmacon, Cat#J-009914-05 and J-009914-08). Transfections were performed with OptiMEM (Invitrogen) and Oligofectamine (Invitrogen) as previously described 57. For evaluation of effect on androgen signaling, cells were first hormone starved and treated with indicated siRNAs against ASH2L or MLL. After 48 hours, cells were treated with 1 nM R1881 for 3, 6 and 24 hrs for qPCR prior to RNA isolation. qPCR was performed essentially as described using Power SYBR Green Mastermix (Applied Biosystems) on an Applied Biosystems 7300 Real Time PCR system for quantification of ASH2L and MLL knockdown and PSA expression 43. Primer sequences are in Table 13.

### FOXA1

FOXA1 wildtype and FOXA1 mutants (S453fs, G87R, L388M, L455M and F400I) were cloned and inserted into pCDH (System Biosciences), which has been modified to express an Nterminal FLAG tag and puromycin resistance. Lentiviruses were generated in 293FT cells using the ViraPower Lentiviral Expression System (Invitrogen). LNCaP cells were infected with the generated viruses (or empty control virus) and stable pooled populations were selected with puromycin. Expression was confirmed by western blotting with anti-FLAG antibody (Sigma) or qPCR for FOXA1 expression as above, and FOXA1 primers are in Table 13. qPCR experiments were performed in triplicate, and FOXA1 expression was normalized to GAPDH. For proliferation, cells were starved for 24 hours in phenol red-free media with 1% charcoal-dextran stripped serum, and grown in media with 1% charcoal-dextran stripped serum+/−10 nM DHT. Relative cell numbers were measured in quadruplicate by WST-1 assays at indicated time points following the manufacturer's instructions (Roche).

Gene expression microarray analysis was performed as above, using LnCAP cells expressing empty vector, FOXA1 wild type, or FOXA1 mutants as just described. Cells were starved in 1% charcoal stripped media for 24 hours and treated with 10 nM DHT or vehicle for 48 hours. RNA was isolated using Qiazol. All samples were hybridized against vehicle stimulated vector control in duplicate. Probes not passing filtering in both duplicate hybridizations were excluded from further analysis, and remaining probes were averaged. For each set of FOXA1 wildtype or mutant hybridization (duplicates of DHT and vehicle treated), DHT vs. vehicle stimulated ratios for each probe passing filtering on all four arrays were computed. Probes were filtered to include only those with average LogRatio (converted to log base 2) of >1 or <−1 in the DHT vs. vehicle stimulated pair. Clustering of probes using centroid linkage clustering was performed using Cluster 3.0 and heatmaps were generated using JavaTreeview.

In parallel, FOXA1 wildtype and FOXA1 mutant (S453fs; resulting from chr14:37130381insCC observed in T12) ORFs were generated by gene synthesis (Blue Heron) and cloned into the pLL_IRES_GFP lentival vector. Lentiviruses (and pLL_IRES_GFP expressing LACZ as control) were generated by the University of Michigan Vector Core. LNCaP cells were transduced in the presence of 4 g/mL polybrene (Sigma). After 72 hours, GFP+ cells were sorted at the University of Michigan flow cytometry core. Cells were genotyped to confirm identify. GFP fluorescence was monitored every other day. Soft agar colony forming assays were performed as described 58, except colonies were counted and photographed without staining.

For xenograft experiments, four week-old male SCID C.B17 mice were procured from a mice breeding colony at University of Michigan. Mice were anesthetized using a cocktail of xylazine (80 mg/kg IP) and ketamine (10 mg/kg IP) for chemical restraint. Indicated LNCaP cells (2 million cells per implantation site) as above (or parental LNCaP cells) were suspended in 100 ul of 1×PBA with 20% high concentration Matrigel (BD Biosciences). Cells were implanted subcutaneously on both sides into the flank region. Tumor growth was monitored bi-weekly by using digital calipers in LNCaP FOXA1 wildtype (n=9), LNCaP FOXA1 S453fs (n=10) and parental LNCaP (n=6) groups. Tumor volumes were calculated using the formula (r/6) (L×W2), where L=length of tumor and W=width. All procedures involving mice were approved by the University Committee on Use and Care of Animals (UCUCA) at the University of Michigan and conform to their relevant regulatory standards.

### DLX1

For DLX1 immunoblotting, prostate tissues were homogenized in NP40 lysis buffer containing 50 mm Tris-HCl (pH 7.4), 1% NP40 (Sigma), and complete proteinase inhibitor mixture (Roche). Western blotting with ten micrograms of each protein extract was performed as above. Transferred membrane was incubated for 1 h in blocking buffer and over-night with anti-DLX1 rabbit polyclonal antibody (PTG laboratory, #13046-1-AP, 1:1000 dilution). After washing three times with TBS-T buffer, the membrane was incubated with horseradish peroxidase-linked donkey anti-rabbit IgG antibody (GE Healthcare, 1:5,000) for 1 h at room temperature prior to visualization by enhanced chemiluminescence (GE Healthcare). To monitor equal loading, the membrane was re-probed with anti-β-Actin mouse monoclonal antibody (1:30,000 dilution; Sigma, #A5316).

qPCR was performed on 10 benign prostate tissues (included in gene-expression profiling), 55 localized prostate cancers (including 32 samples subjected to gene-expression profiling) and 7 CRPCs (including 6 samples subjected to gene-expression profiling) as above. The amount of DLX1 in each sample was normalized to the average of GAPDH and HMBS for each sample. Primers for DLX1 are given in Table 13; GAPDH and HMBS primers were as described (Vandesompele, J. et al. Genome Biol 3, RESEARCH0034 (2002)). All oligonucleotide primers were synthesized by Integrated DNA Technologies.

### B. Results

The Mutational Landscape of CRPC by Whole Exome Sequencing The exomes of 50 lethal CRPCs, including three derived from different sites from the same patient, and eleven treatment naïve high grade localized prostate cancers (Table 1), with corresponding paired normal tissue, were sequenced using the SureSelect Enrichment System and next-generation sequencing on the Illumina GAIIx and HiSeq 2000 platforms. In total 25,525,520,145 bases, with an average 116-fold coverage of each targeted base per tissue sample, and 91.78% of annotated targeted bases with sufficient coverage to call somatic mutations were generated (Tables 2&3). A total of 3,875 high confidence protein-altering somatic mutations were identified in 3,044 genes (out of ˜19,365 targeted coding genes) among the 61 tumors, including 3,169 missense, 203 nonsense, 68 splice site mutations, and 435 indels. Neutral mutations were also identified, including 2,179 intronic and 1,225 synonymous. Confirmation as somatic by Sanger sequencing of candidate point mutations (219/227, 96%) and indels (16/16, 100%), confirmed the stringency of the somatic mutation calling algorithm (FIG. 5). The estimated average tumor content for CRPC and localized prostate cancer samples was 68% (range 40%-100%) and 56% (range 35%-77%), respectively (p=0.04) (FIG. 6).

Of the 3,875 identified non-synonymous somatic mutations, only 54 somatic SNVs are present in COSMIC, including, but not limited to, one each in SPOP, ARIDIA, and KRAS (G12V), two in TTN, three each in APC, CTNNB1, and RB1 and 23 in TP53. The average number of mutations per tumor was 46.6 over an average of 28.7 Mb of annotated targeted bases in each exome with sufficient coverage to call somatic mutations (range 13-100 somatic mutations per sample, FIG. 7), excluding three samples with outlier number of mutations: WA56 (169 mutations), WA48 (238 mutations) and WA16 (731 mutations).

Rare CRPC xenografts with outlier number of mutations were observed by Kumar et al. (Proc Natl Acad Sci US A 108, 17087-17092 (2011), in one case likely due to a mutation in the mismatch repair gene MSH6 previously associated with Lynch syndrome. WA16 harbored a somatic, focal homozygous deletion in the mismatch repair gene MSH2, while WA48 harbored a somatic homozygous deletion of a −2 MB region on chr 13 harboring BRCA2 (FIG. 8).

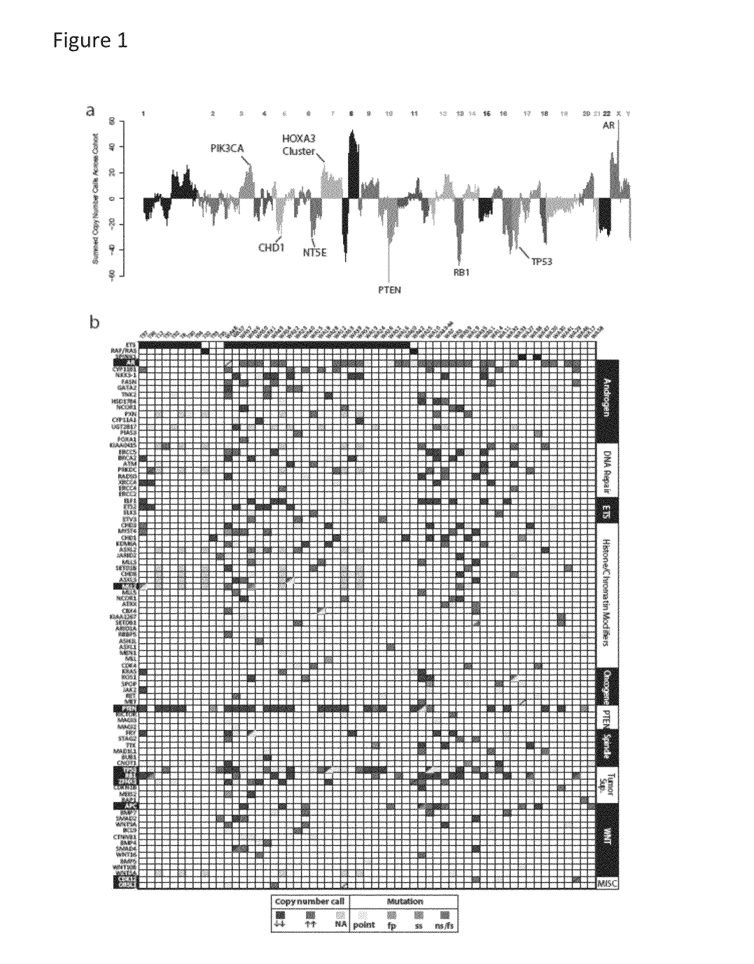

In the cohort, the mutation rate for localized prostate cancers (0.93/Mb) was consistent with the rate observed in the whole genome sequencing of seven localized prostate tumors (0.9/Mb) (Berger, M. F. et al. cancer. Nature 470, 214-220 (2011)) and with the low reported rates in other targeted studies of localized prostate cancer (0.33 and 0.31/Mb) (Kan, Z. et al. Nature 466, 869-873 (2010); Tomlins, S. A. et al. Eur Urol 56, 275-286 (2009)). The mutation rate for heavily treated CRPC (2.00/Mb) was only two-fold higher than that of the localized tumors. Additional observations on the prostate cancer mutation signature, including the mutational spectrum of CRPC (FIG. 9), confirmation of the monoclonal origin of lethal CRPC (FIG. 10), and overlap with mutations observed by Berger et al (Nature 470, 214-220 (2011)) and Kumar et al. (Proc Natl Acad Sci USA 108, 17087-17092 (2011)), are provided below. Mutations effecting the same residue (F133) in SPOP and splice site mutations involving CHD1 were observed (see FIG. 1).

It was recently showed that exome sequencing can be used to identify somatic copy number alterations in cancer (Lonigro, R. J. et al. Detection of somatic copy number alterations in cancer using targeted exome capture sequencing. Neoplasia 13, 1019-1025 (2011)), hence this methodology was applied to all profiled CRPC and localized prostate cancer samples (see FIG. 11). As shown in FIG. 1a, recurrent aberrations previously associated with prostate cancer development and progression were identified, including broad losses of 1p, 8p and 6q, and gains of 1q, 3q, 7q and 8q, and deletions between TMPRSS2 and ERG (in cases with TMPRSS2:ERG fusions through deletion)-4, 18-20. Profiles for all samples, CRPC and localized prostate cancer are shown in FIG. 12.

To further characterize the landscape of CRPC, array CGH based copy number profiling and gene expression profiling of a matched cohort of 28 benign prostate tissues, 59 localized prostate cancers (including 3 subjected to exome sequencing) and 35 CRPCs (including 31/35 subjected to exome sequencing was performed; Table 4), as well as transcriptome sequencing of 11 prostate cancer cell lines (primarily CRPC, see Table 5) to identify potential somatic variants. Copy number and gene expression profiles were uploaded into Oncomine for automated data processing, analysis and visualization, and are also available for exploration. aCGH profiles were similar to copy number analysis by exome sequencing and to other prostate cancer profiling studies available in Oncomine (FIG. 1a and FIG. 11). Global gene expression profiles were similar to previous studies (analyses available in Oncomine), although as described below, DLX1, a gene not monitored in most previous microarray studies, was identified to be the most differentially expressed gene between benign prostate tissue and localized prostate cancer (FIG. 14a), and overexpression of DLX1 in prostate cancer and CRPC was confirmed by qPCR and western blotting (FIG. 14b&c). Finally, transcriptome sequencing for prostate cancer cell lines was performed and high-stringency filters were used to identify likely somatic variants (see Tables 8-10 and Methods). As described below, this analysis identified additional mutations in genes prioritized by exome sequencing.

Nine genes were identified that were significantly mutated (point mutations and indels) at a false discovery rate (FDR) of <0.10 were identified (FIG. 1b, Tables 11&4), six of which have been reported as recurrently mutated in prostate cancer: TP53 (mutated in 23 unique samples, 39.7%, q<1.0×10⁻⁶), AR (5 samples, 8.6%, q<0.0002), ZFHX3 (8 samples, 13.8%, q<0.0012) (Sun, X. et al. Nat Genet. 37, 407-412 (2005), RB1 (6 samples, 10.3%, q<0.0012), PTEN (6 samples, 10.3%, q<0.0013) and APC (6 samples, 10.3%, q<0.0015). Three other significantly mutated genes do not have described roles in prostate cancer: MLL2 (5 samples, 8.6%, q<0.0169), OR5L1 (2 samples, 3.4%, q<0.0573) and CDK2 (3 samples, 5.2%, q<0.0859).

MLL2 encodes a H3K4-specific histone methyltransferase (Varier, R. A. & Timmers, H. T. Biochim Biophys Acta 1815, 75-89 (2011)) that is recurrently mutated in diffuse large B-cell lymphoma (Morin, R. D. et al. Nature 476, 298-303 (2011)), urothelial carcinoma (Gui, Y. et al. Nat Genet. 43, 875-878 (2011)) and medulloblastoma (Parsons, D. W. et al. Science 331, 435-439 (2011)), and is a direct coactivator of the estrogen receptor (Mo et al., J Biol Chem 281, 15714-15720 (2006)). CDK12, which encodes a transcription elongation-associated C-terminal repeat domain (CTD) kinase (Bartkowiak, B. et al. Genes Dev 24, 2303-2316 (2010)), was recently identified as one of nine significantly mutated genes in ovarian serous carcinoma (Nature 474, 609-615 (2011)), and silencing of CDK2 has previously been shown to cause resistance to tamoxifen and estrogen deprivation in ER-dependent breast cancer models (lorns et al., Carcinogenesis 30, 1696-1701 (2009)), indicating a potential role in endocrine resistance in CRPC. OR5L1 is an olfactory gene that exhibits a higher than average mutation rate as a result of its late replication, arguing against a role in cancer (Stamatoyannopoulos, J. A. et al. Nat Genet. 41, 393-395 (2009)).

88 significantly mutated canonical pathways out of 880 considered were identified (see Methods & Table 10), including 49 with substantial contributions from the nine significantly mutated genes. For example, the ‘WNT signaling’ KEGG pathway was identified as significantly mutated (57 somatic mutations, 38 samples, q-value=1E-6). Half of these mutations occurred in genes other than TP53 and APC, including three missense mutations in CTNNB1 and a splice site mutation in MYC. Additionally, WA57 harbored concurrent nonsense mutation (W509*) and high-level copy loss in SMAD4, a gene which has recently been described as controlling lethal metastasis in CRPC (Ding, Z. et al. Nature 470, 269-273 (2011)).

The matched somatic point mutation and exome copy number data was used to identify altered subnetworks in a large protein-protein interaction network using HOTNET (Vandin et al., J Comput Biol 18, 507-522 (2011). This analysis identified 14 known KEGG pathways or protein complexes (Table 11) as significantly mutated in CRPC, including a PTEN interaction network, which was altered in 81% of samples (FIG. 15). While 48% of CRPC samples have PTEN mutations, 33% of CRPC samples have mutations in a protein that directly interacts with PTEN, indicating an even broader role for PTEN in prostate cancer pathogenesis and indicating that mutational status of numerous genes may be required for stratification of therapies targeting the PTEN pathway. For example, R215W mutation was identified in WA57 of MA G2, which encodes a PTEN interacting protein and was reported as recurrently deregulated by rearrangements by Berger et al (Nature 470, 214-220 (2011).) Similarly, while most members of the PTEN interacting protein network were altered as a result of copy number changes, two genes exhibited recurrent somatic point mutations: MAGI3 and HDAC11 (each mutated in 4% of CRPC samples), indicating potential roles in prostate cancer progression.

In addition, candidate driver mutations were identified in genes associated with androgen receptor signaling (see below), DNA damage response, histone/chromatin modification (see below), the spindle checkpoint, and classical tumor suppressors and oncogenes (FIG. 1b). For example, two deleterious mutations were identified in PRKDC (11137fs and E640*), which encodes the catalytic subunit of the DNA-dependent protein kinase involved in DNA double strand break repair and recombination, in patient T96, who had an extremely aggressive localized prostate cancer. Similarly, three CRPC samples were identified with mutations in FRY (R100C in WA32, 11480T in WA56, S25 100N in WA57), the homologue of the Drosophila gene Furry that encodes a microtubule binding protein required for precise chromosome alignment (Chiba et al., Curr Biol 19, 675-681 (2009)). Mutations in FRY may promote chromosomal instability in CRPC or result from selection during treatment with docetaxel (a microtubule binding agent), a standard therapy for men with CRPC. Finally, a KRAS G12V mutation was identified in WA42 (ETS−), consistent with previous reports of rare RAF and RAS family aberrations in ETS-prostate cancers (Palanisamy, N. et al. Nat Med 16, 793-798 (2010); Wang, X. S. et al. Cancer Discov 1, 35-43 (2011)).

To identify potential drivers of CRPC, genes with recurrent highlevel gains or losses present in peaks of global copy number change were compared to genes with identified mutations (FIG. 16). For example, AR on chr X had the maximum copy number sum (57), with 25 samples showing high-level copy number gain. Likewise PTEN on chr 10 had the minimum copy number sum (−64), with 25 samples showing high-level copy number loss. Both genes also harbored recurrent somatic mutations (FIG. 1B), supporting the validity of this approach. The peak of copy number loss on chr 5q21 (FIGS. 1a, 2a and 15) that harbors CHD1, which encodes an ATPdependent chromatin-remodeling enzyme that was reported as recurrently deregulated in 3 of 7 prostate cancer genomes by Berger et al. (one somatic splice-site mutation and two rearrangements) were identified (Berger et al., supra). Three CRPCs (WA7, WA19 and WA10), all of which were ETS-, showed focal high-level copy loss of CHD1. Additionally, a single CHD1 mutation, a splice site mutation (e28+1) in WA27 (which is ETS−) was identified. One additional ETS− localized prostate cancer (T93) and two ETS+ CRPCs (WA12 and WA60), showed focal single copy losses involving CHD1. Finally, by aCGH analysis of the matched cohort, the focal deletions of WA7, WA19 and WA10 were confirmed and three additional localized PCs with focal, high copy loss of CHD1 (FIG. 17a) were identified. Thus, focal deletion/mutation of CHD1 was observed in 10/119 (8%) prostate cancers in the total cohort (exome and aCGH), with focal deletion/mutation of CHD1 (CHD1−) being significantly associated with ETS− status (two sided Fisher's exact test, p=0.02). The correlation of CHD1 gene expression and genomic CHD1− status in the cohort is shown in FIG. 13b.

The association between CHD1 and ETS status in prostate cancer was analyzed using three prostate cancer aCGH studies totaling an additional 331 cancers using Oncomine Powertools. Each study showed a peak of copy number loss on 5q21, and in each study, all cancers with focal deletions of CHD1 were ETS− (15 of 331 total, 4%, FIGS. 13&18). For example, in the Taylor et al. study with 218 prostate cancers 4, 9 were identified with focal deletions of CHD1, all of which were ETS− (FIG. 2b). Thus, in total, 25 of 450 prostate cancers were identified as CHD1− in DNA based studies, 23 of which were ETS− (two sided Fisher's exact test, p=0.0002). Finally, the association of CHD1 and ETS status were analyzed by gene expression profiling using an additional 9 microarray studies (totaling 504 prostate cancers) available on Oncomine. 25 of 504 (5%) prostate cancers with outlier underexpression of CHD1 were identified, all of which were ETS− as assessed by lack of outlier overexpression of ERG, ETV1, ETV4 or ETV5 (p<0.0001, two sided Fisher's exact test), with the Glinsky et al. and Lapionte et al. datasets (Glinsky et al., J Clin Invest 113, 913-923 (2004); Lapointe, J. et al. Proc Natl Acad Sci USA 101, 811-816 (2004)) shown in FIG. 2c. Thus, in total, across 13 DNA and RNA based studies, 50 of 954 prostate cancers were identified as being CHD1−, 48 of which (96%) were ETS− (p<0.0001, two sided Fisher's exact test, FIG. 2d). Of note, CHD1-prostate cancers show some overlap with SPINK1+ cancers, indicating that these are not mutually exclusive classes of ETS− tumor.

CHD1 is frequently deleted in prostate cancer (exclusively in ETS− cancers in Liu et al.'s cohort) and has tumor suppressor properties, confirming our observations (Liu, W. et al. Oncogene (2011); Huang, S. et al. Oncogene (2011)). Additionally, other tumors with focal deletions involving other genes at 5q21, including PJA2 (high-level copy loss in T65 and T53, and Y505C in WA53) were identified, indicating the existence of other potential drivers at 5q21 (FIGS. 17a,c&d). The integrated analysis identifies deletion or mutation of CHD1 as defining a novel subtype (CHD1−) of ETS− prostate cancer.

### Deletion and Mutation of ETS2 in Prostate Cancer

The dataset was evaluated for aberrations in additional ETS family members. The majority of ETS+ CRPCs retained marked over-expression of the rearranged ETS gene (ERG, ETV1 or ETV5), consistent with active androgen signaling in the majority of men with lethal CRPC (FIG. 137). However, those with copy number loss of the −3 Mb intervening region between TMPRSS2 and ERG on chromosome 21 (TMPRSS2:ERG fusions through deletion), but loss of ERG gene expression (such as WA22 and WA24) represent true androgen signaling independent CRPC (FIG. 17b and FIG. 2e).

Two CRPC samples with deleterious mutations in ETV3, (P327fs in WA56 and W38* in WA26, both ETS+), which does not have a described role in prostate cancer were identified. In addition, the mutation of ETS2 (R437C) in WA30 (ETS−) was identified. ETS2 binds to a similar DNA binding motif as ERG39 and is located immediately telomeric to ERG (head-to-head orientation) in the commonly deleted region in TMPRSS2:ERG fusions through deletion. Given observations that prostate cancers with ERG rearrangements through deletion may have a more aggressive clinical course than those with ERG rearrangements through insertion, it was contemplated that this intervening region may harbor additional tumor suppressors, including ETS218 (Perner, S. et al. Cancer Res 66, 8337-8341 (2006); Yoshimoto, M. et al. Neoplasia 8, 465-469 (2006)). The copy number profiling data generated here demonstrates that multiple ERG rearrangement positive CRPCs show focal deletions extending telomeric from ERG (FIG. 2e), consistent with previous observations in the LuCap35 xenograft and the NCI-H660 prostate cancer cell line (small cell ERG+) (Demichelis, F. et al. Genes Chromosomes Cancer 48, 366-380 (2009); Mertz, K. D. et al. Neoplasia 9, 200-206 (2007)). WA31 (ERG+ through insertion) shows a focal, high copy number loss of ETS2, and the gene expression data demonstrates decreased ETS2 expression in localized cancer and CRPC, with the lowest expression in WA31 (FIG. 19a). Finally, the R437c mutation in ETS2 occurs in the ETS domain at a DNA contacting residue conserved in class I ETS transcription factors39, which include all ETS genes known to be involved in gene fusions in prostate cancer (FIG. 2f). Hence, to investigate whether ETS2 is a tumor suppressor deregulated through both deletion and mutation in prostate cancer, VCaP cells (a prostate cancer cell line that endogenously expresses TMPRSS2:ERG) that stably over-express wild type ETS2 (VCaP ETS2 wt), ETS2 R437c (VCaP ETS2 R437C) or LACZ as control (VCaP LACZ) were generated (FIG. 19b). As shown in FIG. 2g, VCaP ETS2 wt showed decreased cell migration (in a Boyden chamber migration assay) compared to VCaP LACZ (0.6 fold, p=1.0E-5), while VCaP ETS2 R437c showed increased migration compared to VCaP LACZ (1.2 fold p=0.03) and VCaP ETS2 wt (2.0 fold, p=7.1E-7). Effects on cell invasion were even more pronounced (FIG. 2g), with expression of ETS2 wt significantly decreasing invasion compared to LACZ (0.4 fold, p=2.1E-5), while expression of ETS2 R437c resulted in significantly increased invasion (1.7 fold, p=0.006). Lastly, while VCaP ETS2 R437c showed only minimally increased cell proliferation compared to VCaP LACZ (1.07 fold, p=0.004), VCaP ETS2 wt showed markedly decreased cell proliferation compared to VCaP LACZ (0.65 fold, p=8.2E-9). Together, these results support ETS2 as a prostate cancer tumor suppressor that can be deregulated through deletion (resulting in both increased invasion and proliferation) or mutation (predominantly increasing invasion). As ETS genes involved in gene fusions have been shown to dramatically impact cell invasion (Tomlins, S. A. et al. Neoplasia 10, 177-188 (2008); Hollenhorst, P. C. et al. Genes Dev 25, 2147-2157 (2011)), ETS2 may directly compete with other ETS transcription factors for binding to target.

Identification of Chromatin/Histone Modifying Genes Mutated in CRPC that Interact with Androgen Receptor

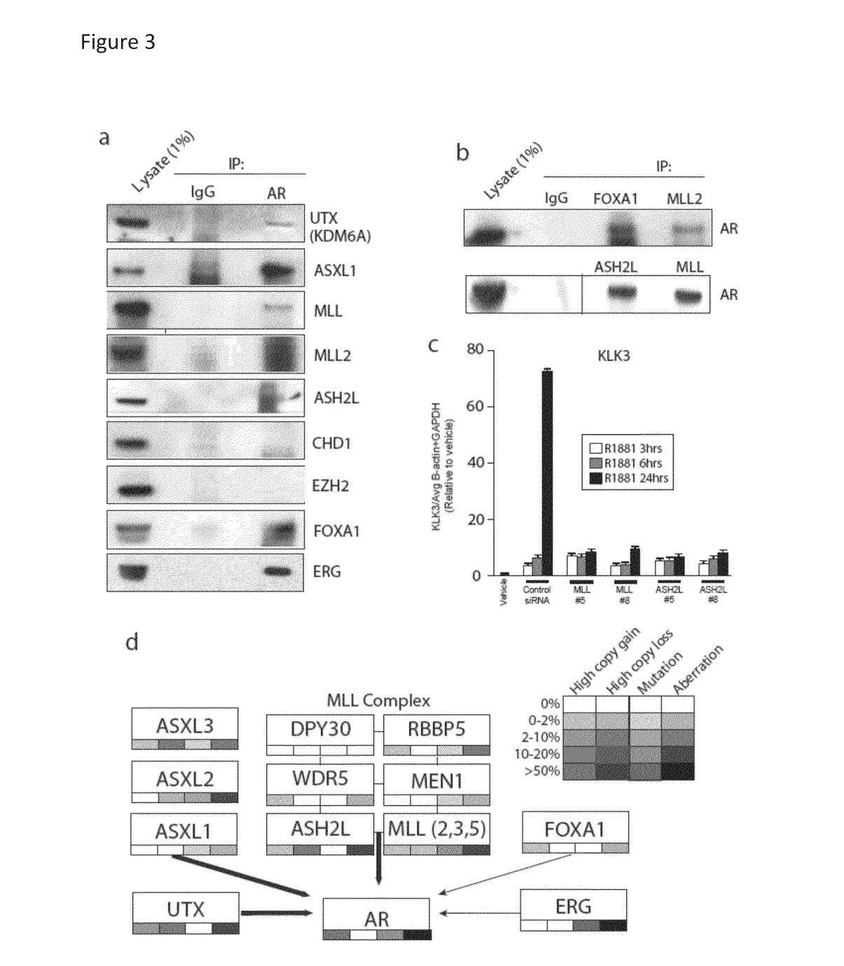

In addition to CHD1, which defines an ETS− subset of prostate cancer, the integrated analysis identified mutations and copy number aberrations in multiple other genes involved in chromatin/histone modification (FIG. 1), including MLL2, which was the 7th ranked significantly mutated gene in the data set. The MLL genes (MLL, MLL2 and others) encode histone methyltransferases that function in multi-protein complexes that mediate H3K4 methylation required for epigenetic transcriptional activation (Varier, R. A. & Timmers, Biochim Biophys Acta 1815, 75-89 (2011)). In addition to MLL2, a frame preserving indel in MLL (Q1815fp in WA28) and deleterious mutations in MLL3 (R1742fs in WA18 and F4463fs in WA56) and MLL5 (E1397fs in WA57) were identified. In total, 10 of 58 (17.2%) of all samples harbored mutations in an MLL gene. Additionally, while the MLL proteins possess catalytic activity through a SET domain, MLL and MLL2 function as part of a multi-protein complex that includes ASH2L, RBBP5, WDR5 and MEN1 (menin)-all of which harbor varying levels of aberration in CRPC (see below and FIG. 3).

Additional deregulated epigenetic modifiers identified included the polycomb group gene ASXL2 which was the 17th significantly ranked significantly mutated gene in the data set (p=3.4E-4) and was mutated in 4 samples, with 3 samples harboring nonsense mutations (Y1163* in WA31, Q1104* in WA56 and Q172* in WA23) (FIG. 1B). Single samples with nonsense mutations in ASXL1 (P749fs in WA52) and ASXL3 (L2240V and R2248* in WA22) were also identified. ASXL1 is recurrently mutated in myeloid disorders, predominantly through frameshift mutations in the last exon45, the same exon affected by the P749fs mutation observed in WA52. Similarly, although UTX (KDM6A), which encodes a histone H3K27 demethylase that complexes with MLL321, is located in a broad region of copy number gain on chr X, it is located at a local copy number minimum, and two samples (WA28 and WA40) show focal high copy loss (FIG. 1b). UTX has been shown to be mutated in a number of cancers including renal carcinoma and urothelial carcinoma (Varier, supra; Dalgliesh, G. L. et al. Nature 463, 360-363 (2010); van Haaften, G. et al. Nat Genet. 41, 521-523 (2009)). Additional putative somatic mutations in histone/chromatin remodelers were identified through transcriptome sequencing of prostate cancer cell lines. Besides CHD1, which shows deregulation in both localized prostate cancer and CRPC (FIG. 2 and FIGS. 17&18), mutations of other chromatin/histone remodeling genes were infrequent in localized prostate cancer and concentrated in a single sample (e.g. MYST4 E1501*, MLL2 A4223fs/D2056G and CHD3 M576fs all in T97, FIG. 1b). The present invention is not limited to a particular mechanism. Indeed, an understanding of the mechanism is not necessary to practice the present invention. Nonetheless, it is contemplated that given the importance of androgen signaling to progression to CRPC and the selection for deregulation of AR signaling components in CRPC (e.g., high-level copy gains or mutations in AR in 30/48 CRPCs and 0/11 localized prostate cancers in the cohort), it was contemplated that the mutated chromatin/histone remodelers identified may play a direct role in AR signaling through interaction with AR.

Thus, AR was immunoprecipitated from VCaP cells (ERG+CRPC that maintains active AR signaling) and blotted for members of the MLL complex (MLL2, MLL, ASH2L), UTX, ASXL1 and CHD1. FOXA1, a known direct interacting cofactor of AR (Yu, X. et al. Ann N Y Acad Sci 1061, 77-93 (2005)), and EZH2 (a H3K27 histone methyltransferase over-expressed in CRPC), were also evaluated as positive and negative controls, respectively. As shown in FIG. 3a, members of the MLL complex (MLL, MLL2 and ASH2L), UTX and ASXL1 all endogenously interact with AR, while interaction between CHD1 and AR was not observed. Interaction between AR and MLL, MLL2, ASH2L and FOXA1 (as positive control) was confirmed by reverse immunoprecipitation in VCaP cells (FIG. 3b and FIG. 20a). As the MLL complex is implicated in epigenetic transcriptional activation, its role in AR signaling was analyzed. RNA interference of MLL or ASH2L using independent siRNAs (FIG. 20b) significantly inhibited AR signaling, as assessed by inhibition of R1881 (synthetic androgen) stimulation of KLK3 (PSA) expression, with two siRNAs against MLL and ASH2L each inhibiting KLK3 expression at 24 hours by >7.5 fold (each p<0.001) (FIG. 3c). Together, the data show that mutation and copy number alteration of histone modifiers are common in CRPC, and that aberrations in AR and proteins that physically interact with AR, including chromatin/histone remodelers, ETS genes (exemplified by ERG, which directly interacts with AR50) and known AR co-regulators including FOXA1 (see below), drive prostate cancer development and progression to CRPC (FIG. 3d).

Disruption of FOXA1 in Prostate Cancer Through Mutation Given the central role of AR signaling in CRPC, and the selection for aberrations in AR occurring in CRPC, the identification of a somatic 2 bp insertion in FOXA1 (S453fs) in the localized prostate cancer sample T12 and transcriptome sequencing identification of 340fs and P358fs indels in DU-145 and LAPC-4, respectively, were investigated as FOXA1 has a well described role in AR signaling (Gao, N. et al. Mol Endocrinol 17, 1484-1507 (2003); Wang, Q. et al. Mol Cell 27, 380-392 (2007); Wang, Q. et al. Cell 138, 245-256 (2009); Lupien, M. et al. Cell 132, 958-970 (2008); Sahu, B. et al. Embo J 30, 3962-3976 (2011); Zhang, C. et al. Cancer Res 71, 6738-6748 (2011)). Thus, 101 localized and 46 CRPCs (including all foci from all CRPC samples subjected to exome sequencing) prostate cancer samples were screened. Somatic mutations of FOXA1 were identified in 5 of 147 (3.4%) prostate cancers (FIG. 4a), including 4 localized prostate cancers (including the S453 insertion identified in T12 in the exome sequencing, G87R in T68, L388M in T70 and L455M in T18086), and 1 CRPC (F400I in WA40, a small cell CRPC, which was a different metastatic focus from that used for exome sequencing. Four of the 5 mutations, as well as both FOXA1 indels identified in the transcriptome screen, occurred in the C-terminal transactivating domain (last 130 AA) (FIG. 4a).

Exploring the role of FOXA1 in androgen signaling, Wang et al. recently reported that down-regulation of FOXA1 (by siRNA) in LNCaP cells triggers dramatic reprogramming of the hormonal response and enhances entrance to S phase, and decreased expression of FOXA1 is associated with poor outcome in CRPC57. In contrast, Gerhardt et al. reported that FOXA1 is over-expressed in CRPC and siRNA knockdown of FOXA1 results in decreased growth of LNCaP cells58.

Thus, stable LNCaP cells expressing empty vector (LNCaP vector), wild type FOXA1 (FOXA1 wt) and the five FOXA1 mutants were generated as N-terminal FLAG fusions. Western blot and QPCR analyses confirmed equivalent levels of expression of each FOXA1 construct (FIG. 4b and FIG. 21a). The S453fs insertion allele encodes a protein with a predicted molecular weight 49 kDa, similar to wild type FOXA1 (49.2 kDa).

In LNCaP cells grown in the presence of 10 nM DHT, all FOXA1 mutants, as well as FOXA1 wt, showed significantly increased cell proliferation compared to LNCaP vector (p=0.006 for FOXA1 F400I, p<0.001 for all comparisons to LNCaP vector), while only FOXA1 L388M showed significantly increased growth compared to FOXA1 wt (p=0.005). Expression of FOXA1 wt or mutants had no significant effect on LNCaP proliferation in the absence of androgen (FIG. 21b).

Given the role of FOXA1 as a cofactor for AR signaling, and the reported ability of FOXA1 to repress portions of the AR program (Sahu et al., supra; Wang, D. et al. Nature 474, 390-394 (2011)), gene expression profiling from LNCaP vector, FOXA1 wt and FOXA1 mutant cells stimulated with vehicle or 10 nM DHT for 48 hours was performed. Focusing on the AR mediated program, 352 probes showing ≧2 fold over-expression and 262 probes showing ≦−2 fold underexpression upon DHT stimulation in LNCaP vector cells were identified (FIG. 4d).

Generalized repression of AR signaling in LNCaP FOXA1 wt and FOXA1 mutant cells, with 81% of these DHT stimulated probes in LNCaP vector cells showing <1.5 (for overexpressed probes) or >−1.5 fold change (for under-expressed probes) in LNCaP FOXA1 wt cells was observed. In contrast, only 6% of probes showed enhanced expression in LNCaP FOXA1 wt cells (>2 or <−2 fold change). Similar effects were observed in FOXA1 mutant cell lines with an average of 59% repressed probes (range 43-73%) vs. 23% enhanced probes (range 5-39%). The stimulation of KLK2, KLK3 (PSA) and NKX3-1 were not significantly repressed by FOXA1 wt or FOXA1 mutants.

Based on the effects of FOXA1 wt and FOXA1 mutants on proliferation, LNCaP cells stably expressing 3×HA-N-terminally tagged FOXA1 wt, FOXA1 S453fs, or LACZ (as control) were generated through a different lentivirus construct. These cells were used for soft agar colony forming assays, and as shown in FIG. 4e, both FOXA1 wt and FOXA1 S453fs formed significantly more colonies than LACZ cells (p<0.05 for each) in the presence of 1 nM of the synthetic androgen R1881. Finally, parental LNCaP, LNCaP FOXA1 wt and LNCaP FOXA1 S453fs cells were used in xenograft experiments. As shown in FIG. 4f, by 20 days, both LNCaP FOXA1 wt and FOXA1 S453fs cells formed significantly larger tumors than parental LNCaP cells. Taken together, mutations in the AR collaborating factor FOXA1, which occur in both untreated localized prostate cancer and CRPC, and promote cell growth and repress AR signaling, with similar effects to over-expression of wild type FOXA1 were identified.

### Mutational Spectrum of Castrate Resistant Prostate Cancer

Based on the low mutation rate, the metastatic prostate cancer mutation signature likely does not reflect exposure to tobacco carcinogens, UV light or mutagenic alkylating chemotherapy (Greenman, C. et al. Nature 446, 153-158 (2007), consistent with lack of etiologic associations with prostate cancer. The metastatic prostate cancer mutation signature was enriched for C to T transitions at 5′-CG base pairs (30.5% of nonsynonymous mutations) (FIG. 9), similar to the mutational spectrum of ovarian clear cell carcinoma identified by exome sequencing (Jones, S. et al. Science 330, 228-231 (2010)), and gastric (Greenman et al., supra), colorectal (Greenman et al., supra; Sjoblom, T. et al. Science 314, 268-274 (2006); Wood, L. D. et al. Science 318, 1108-1113 (2007)) and pancreatic adenocarcinoma (Jones, S. et al. Science 321, 1801-1806 (2008)), and glioblastoma multiforme (Parsons, D. W. et al. Science 321, 1807-1812 (2008)). Unlike breast (Greenman et al., supra; Wood et al., supra), lung and ovarian carcinoma, and melanoma (Greenman et al., supra), the prostate cancer mutation signature is not enriched for C:G>G:C changes at 5′-TC base pairs. The localized prostate cancer mutation spectrum was almost identical to the spectrum for metastatic prostate cancer (R2=0.974), indicating that heavy treatment does not substantially alter the types of mutations arising in prostate cancer with C to T transitions at 5′-CG being the dominant type of mutation (27.9% of nonsynonymous mutations) in localized and metastatic prostate cancer,

### Sequencing of Different Foci Confirms the Monoclonal Origin of Lethal CRPC

Previously, the clonality of ETS gene fusions and copy number profiles have been used to demonstrate the monoclonal origin of lethal CRPC (Mehra, R. et al. Cancer Res 68, 3584-3590 (2008); Liu, W. et al. Nat Med 15, 559-565 (2009); Holcomb, I. N. et al. Cancer Res 69, 7793-7802 (2009)). To confirm these findings at the mutational level, three foci (bladder [WA43-44], celiac lymph node [WA43-27], and right lung [WA43-71]) from a 52 year old man who died of CRPC 5 years after initial treatment with radical prostatectomy, which demonstrated high-grade (Gleason score 9), organ confined disease with focally positive margins, and subsequent treatment with anti-androgen therapy, external beam radiation to the tumor bed, and numerous chemotherapeutics were profiled. As shown in FIG. 10, 59 mutations were identified in the bladder, 55 in the celiac lymph node and 47 in the right lung focus; 37 mutations were present in all three foci, including mutations in TP53 and PIK3C2A, consistent with monoclonal origin.

### Comparison of Nonsynonymous Mutations to Previously Published Prostate Cancer Genomes And Exomes

The nonsynonomous mutations were compared to nonsynonymous mutations observed in prostate cancer genomes and exomes, reported by Berger et al. (Nature 470, 214-220 (2011)) and Kumar et al. (Proc Natl Acad Sci USA 108, 17087-17092 (2011)), respectively. Berger et al. recently reported the genomes of seven localized prostate cancers 10, and 26 genes harbored nonsynonymous mutations in both studies, representing significant overlap (26 overlapping genes out of 2,485 genes harboring nonsynonymous mutations in this study, [excluding WA43-27, WA43-71, and WA16] and 105 genes harboring nonsynonymous mutations in Berger et al., out of 19,365 total genes sequenced, Fisher's exact test, P=0.0006). Both studies identified mutations effecting the same residue (F133) in SPOP, (FIG. 1b), which has been identified in a prostate cancer sample previously (Kan, Z. et al. Nature 466, 869-873 (2010).)). Similarly, CHD1 harbored splice site mutations in a single sample in both studies (FIG. 1b). Kumar et al. recently reported putative somatic mutations from 23 prostate cancer exomes from unmatched xenograft samples (derived from 16 metastatic samples and three high-grade localized cancers) (Kumar et al., supra) and 18 genes harbored recurrent mutations in both studies, representing significant overlap (18 overlapping genes out of 396 genes with recurrent mutations in this study and 131 genes with recurrent mutations in Kumar et al. out of 19,365 total genes, Fisher's exact text, P=2E-10).

### Gene Expression Profiling Identified Over-Expression of DLX1 in Prostate Cancer and CRPC

Matched aCGH and gene expression profiling was performed on 3 localized prostate cancers and 31 metastatic CRPCs subjected to exome sequencing, as well as an additional 28 benign prostate tissues, 56 localized prostate cancers and 4 CRPCs (Table 4). Generated profiles were uploaded into Oncomine for automated data processing, analysis and visualization. Global gene expression profiles for benign prostate tissue, localized prostate cancer and CRPC were similar to previous studies (analyses available in Oncomine), although DLX1, a gene not monitored in most previous microarray studies, was identified as the most differentially expressed gene between benign prostate tissue and localized prostate cancer (FIG. 5a, fold change 22.4, P=7.2E-27), with AMACR (fold change 13.1, P=4.57E-24), which is currently used diagnostically (by immunohistochemistry) as a prostate cancer biomarker, being the second most differentially expressed gene. The differential expression of DLX1 by qPCR in prostate cancer (both localized and CRPC, n=62, median 418) compared to benign prostate tissue (n=10, median 1.0, Mann Whitney test P<0.0001) was confirmed (FIG. 14b). The over-expression of DLX1 was confirmed by western blotting in both localized and CRPC compared to benign tissue (FIG. 14c).

Integration of Exome Sequencing with Transcriptome Sequencing of Prostate Cancer Cell Lines

As transcriptome sequencing has also been used to discover recurrent mutations in cancer (Shah, S. P. et al. N Engl J Med 360, 2719-2729 (2009); Wiegand, K. C. et al. N Engl J Med 363, 1532-1543 (2010))). The transcriptome of 11 prostate cancer cell lines (primarily CRPC, Table 5), was sequenced using the Illumina GAIIx platform, comprising 22,731,390,482 bases, and identified an average of 5,905 known coding polymorphisms and 1,031 novel protein-altering variants (756 point mutations and 275 indels) per sample (Table 12). Given the lack of normal genomic DNA from these cell lines, germline and somatic variants cannot be distinguished. Thus, variants fulfilling one of three high stringency filters were considered as likely somatic mutations: 1) deleterious variants affecting a gene harboring a somatic mutation in the study (Table 6), 2) variants affecting the same nucleotide as a somatic mutation in the study (Table 7), or 3) variants affecting the same nucleotide as a confirmed somatic variant in COSMIC (Table 8).

This integrative approach identified additional variants in TP53, AR and APC, supporting the utility of the analysis. A TP53 R248W variant, present in WA10 and previously reported as Somatic (Nature 455, 1061-1068 (2008)), was identified in the VCaP cell line, while previously reported P223L and V274F somatic variants were identified in DU-145 (Taylor, B. S. et al. Cancer Cell 18, 11-22 (2010).), with a V274G variant present in WA37. A confirmed somatic TP53 variant R175H was identified in both WA30 and LAPC-4, consistent with previous reports (Table 8) (Nature 455, 1061-1068 (2008)). Finally, a Y234H confirmed somatic variant (predicted to be damaging) was also present in C4-2B (Table 8). This approach also identified additional mutations in AR, including additional T878A mutations, which has been reported as frequently mutated in CRPC (Gaddipati, J. P. et al. Frequent detection of codon 877 mutation in the androgen receptor gene in advanced prostate cancers. Cancer Res 54, 2861-2864 (1994)), in LNCaP (and its derivative C4-2B) and MDA-PCa-2B (Table 7). MDA-PCa-2B also harbored the previously reported somatic mutation L702H (Zhao, X. Y. et al. Nat Med 6, 703-706 (2000)), while 22RV1 (and its parental line CWR22) harbored a previously confirmed somatic H875Y variant (Tan, J. et al. Mol Endocrinol 11, 450-459 (1997)) (Table 7). Finally, WA40 and WA52 harbored a nonsense mutation (E1576*) and a frameshifting indel, respectively, in APC, while MDA-PCa-2B harbored a missense variant (K1454E) (Table 8) previously confirmed as a somatic mutation in urothelial carcinoma (Kastritis, E. et al. Int J Cancer 124, 103-108 (2009)).

Integrating transcriptome sequencing data also identified recurrent variants in genes not previously identified as being mutated in prostate cancer, including STAG2, MLL3, CNOT1, FAM123B (WTX) and FOXA1 (Tables 8-10). WA32 harbored a R370W somatic mutation in STAG2, and a R370G variant was identified by transcriptome sequencing in LNCaP; mutations in STAG2 have recently been identified as causing aneuploidy across cancer types (Solomon, D. A. et al. Science 333, 1039-1043 (2011)). WA56 and WA50 harbored a frameshifting indel and a likely damaging C4432R mutation, respectively, in MLL3, while MDA-PCa-2B harbored a N4685fs indel. Similarly, frameshifting indels were identified in MLL5 in both WA57 and DU-145. CNOT1, which harbored mutations in three samples from the exome sequencing and one in Berger et al.'s dataset, also had a frame shifting indel in LAPC-4 (F128fs). A confirmed S548F somatic variant in FAM123B (WTX) was identified in T12 and a one bp indel was identified in LNCaP during Sanger sequencing validation efforts. Finally, T12 harbored a somatic 2 bp indel in FOXA1 (S453fs), and transcriptome sequencing identified A340fs and P358fs frame shifting indels in DU-145 and LAPC-4, respectively.

Comparison to Genes Reported as Recurrently Mutated from Single Gene Studies

Through exome sequencing, recurrent mutations in several genes previously reported to be recurrently mutated in prostate cancer were identified, including AR, TP53, and ZFHX3 (each of which was significantly mutated), as well as SPOP; however no mutations were identified in CHEK2, KLF6 or NCOA2 (previously reported to be mutated in prostate cancer). 61 (100%), 51 (84%) and 60 (98%) of the 61 samples had at least 70% of bases with sufficient coverage to call somatic mutations for CHEK2, KLF6 and NCOA2, respectively, indicating that the lack of identified mutations is unlikely to be due to inadequate sequencing, and instead indicating that mutations in these genes may be rare, present in a small population of tumor cells or negatively selected for in CRPC.

TABLE 1

| | | | | | | | Survival | | | |
|---|---|---|---|---|---|---|---|---|---|---|
| | | | ETS/RAF/ | | | | from | Survival | Survival | |
| Sample | Disease | | SPINK1 | Gleason | Serum | Prior | diagnosis | from H | from C | Matched |
| Name | State¹ | Age ² | status³ | score⁴ | PSA⁵ | Treatment ⁶ | (mo)⁷ | (mo)⁷ | (mo)⁷ | GE/aCGH |
| T8 | Localized | 61 | ERG⁺ | 7 | 5.7 | NA | NA | NA | NA | Yes |
| | PC | | | | | | | | | |
| T12 | Localized | 62 | ERG⁺ | 8 | 5.3 | NA | NA | NA | NA | Yes |
| | PC | | | | | | | | | |
| T32 | Localized | 54 | RAF1⁺ | 7 | 10.1 | NA | NA | NA | NA | Yes |
| | PC | | | | | | | | | |
| T90 | Localized | 60 | ERG⁺ | 8 | 27.1 | NA | NA | NA | NA | No |
| | PC | | | | | | | | | |
| T91 | Localized | 55 | ERG⁺ | 9 | 6.5 | NA | NA | NA | NA | No |
| | PC | | | | | | | | | |
| T92 | Localized | 55 | ETV1⁺ | 8 | 5.7 | NA | NA | NA | NA | No |
| | PC | | | | | | | | | |
| T93 | Localized | 71 | No ETS | 9 | 4.2 | NA | NA | NA | NA | No |
| | PC | | | | | | | | | |
| T94 | Localized | 58 | ERG⁺ | 8 | 20.3 | NA | NA | NA | NA | No |
| | PC | | | | | | | | | |
| T95 | Localized | 59 | No ETS | 9 | 15 | NA | NA | NA | NA | No |
| | PC | | | | | | | | | |
| T96 | Localized | 65 | ERG⁺ | 9 | 4.9 | NA | NA | NA | NA | No |
| | PC | | | | | | | | | |
| T97 | Localized | 70 | ERG⁺ | 9 | 15 | NA | NA | NA | NA | No |
| | PC | | | | | | | | | |
| WA3 | CRPC | 53 | No ETS | NA | 1,500 | H, C | 14 | 14 | 9 | Yes |
| WA7 | CRPC | 66 | No ETS | NA | 8,083 | H, C | 64 | 48 | 19 | Yes |
| WA10 | CRPC | 78 | No ETS | NA | 130 | R, H, C, X | 84 | 24 | 7 | Yes |
| WA11 | CRPC | 67 | No ETS | NA | 35 | R, H, X | 39 | 17 | 5 | Yes |
| WA12 | CRPC | 79 | ERG⁺ | NA | 230 | H, C | 60 | 60 | 16 | No |
| | | | (small cell) | (NE diff) | | | | | | |
| WA13 | CRPC | 71 | ERG⁺ | NA | 407 | H, C | 132 | 132 | 23 | Yes |
| WA14 | CRPC | 76 | No ETS | NA | 3,771 | R, H, C | 180 | 96 | 18 | Yes |
| WA15 | CRPC | 74 | (ERG⁺) | NA | 799 | R, H, C | 15 | 7 | 2 | No |
| WA16 | CRPC | 71 | ERG⁺ | NA | 2,300 | R, H, C | 37 | 37 | 15 | Yes |
| WA17 | CRPC | 75 | No ETS | NA | 249 | H, C, X | 33 | 32 | 13 | No |
| WA18 | CRPC | 62 | ERG⁺ | NA | 7,336 | P, R, H, C | 156 | 72 | 15 | Yes |
| WA19 | CRPC | 85 | No ETS | NA | 235 | R, H, C, X | 120 | 120 | 29 | Yes |
| WA20 | CRPC | 67 | No ETS | NA | 181 | P, R, H, C | 96 | 88 | 14 | Yes |
| WA22 | CRPC | 64 | ERG⁺ | NA | 23 | H, C, X | 30 | 30 | 9 | Yes |
| | | | (small cell) | (NE diff) | | | | | | |
| WA23 | CRPC | 73 | ETV1⁺ | NA | 324 | R, H, C, X | 96 | 72 | 44 | Yes |
| WA24 | CRPC | 76 | ERG⁺ | NA | 11 | R, H, C | 54 | 22 | 9 | Yes |
| | | | (small cell) | (NE diff) | | | | | | |
| WA25 | CRPC | 66 | No ETS | NA | 509 | H | 33 | 27 | 0 | Yes |
| WA26 | CRPC | 76 | ETV1⁺ | NA | 2,239 | R, H, C | 156 | 96 | 47 | Yes |
| WA27 | CRPC | 74 | SPINK1⁺ | NA | 1,698 | R, H, C | 105 | 85 | 11 | No |
| WA28 | CRPC | 80 | ERG⁺ | NA | 1,725 | P, R, H, C | 180 | 179 | 61 | Yes |
| WA29 | CRPC | 77 | No ETS | NA | 19 | P, R, H, C | 120 | 120 | 8 | Yes |
| WA30 | CRPC | 71 | No ETS | NA | 1,040 | H, X | 42 | 41 | NA | Yes |
| WA31 | CRPC | 63 | ERG⁺ | NA | 252 | P, R, H, C | 97 | 88 | 73 | Yes |
| WA32 | CRPC | 81 | No ETS | NA | 5,222 | R, H, C, X | 156 | 156 | 26 | Yes |
| WA33 | CRPC | 58 | No ETS | NA | 3,220 | R, H, C | 105 | 105 | 73 | Yes |
| WA35 | CRPC | 71 | No ETS | NA | 72 | R, H, C, X | 109 | 109 | 11 | Yes |
| WA37 | CRPC | 63 | ERG⁺ | NA | 928 | H, C | 41 | 39 | 40 | Yes |
| WA38 | CRPC | 77 | No ETS | NA | 382 | P, H, C, X | 131 | 78 | 32 | No |
| WA39 | CRPC | 71 | ERG⁺ | NA | 269 | P, H, C | 168 | 84 | 123 | Yes |
| WA40 | CRPC | 76 | ERG⁺ | NA | 1,294 | R, H, C, | 47 | 45 | 44 | Yes |
| WA41 | CRPC | 70 | No ETS | NA | 257 | P, H, C | 108 | 78 | 35 | No |
| WA42 | CRPC | 61 | No ETS | NA | 3,776 | H, C | 60 | 42 | 30 | Yes |
| WA43-44 | CRPC | 52 | No ETS | NA | NA | P, R, H, C | 70 | 60 | 40 | No |
| (bladder) | | | | | | | | | | |
| WA43-27 | CRPC | | | | | | | | | |
| (celiac LN) | | | | | | | | | | |
| WA43-71 | CRPC | | | | | | | | | No |
| (right lung) | | | | | | | | | | |
| WA46 | CRPC | 71 | No ETS | NA | 189 | P, R, H, C, X | 69 | 69 | 30 | Yes |
| WA47 | CRPC | 64 | SPINK1⁺ | NA | 12 | H, C, X | 46 | 45 | 43 | Yes |
| WA48 | CRPC | 82 | ERG⁺ | NA | 308 | P, R, H, C | 158 | 105 | 120 | No |
| WA49 | CRPC | 68 | ERG⁺ | NA | 491 | P, R, H, C | 98 | 81 | 73 | No |
| WA50 | CRPC | 78 | ERG⁺ | NA | 74 | P, R, H, C | 192 | 110 | 113 | No |
| WA51 | CRPC | 65 | No ETS | NA | 57 | P, H, C, X | 133 | 122 | 120 | No |
| WA52 | CRPC | 80 | ERG⁺ | NA | 194 | P, H, C, | 160 | 117 | 65 | No |
| WA53 | CRPC | 68 | ERG⁺ | NA | 312 | H, C, X | 56 | 54 | 38 | Yes |
| WA54 | CRPC | 73 | ERG⁺ | NA | 102 | P, R, H, C, X | 162 | 66 | 22 | Yes |
| WA55 | CRPC | 72 | ERG⁺ | NA | 657 | H, C, X | 52 | 50 | 35 | Yes |
| WA56 | CRPC | 79 | ERG⁺ | NA | 353 | P, R, H, C, X | 218 | 82 | 56 | No |
| WA57 | CRPC | 73 | ERG⁺ | NA | 0 | R, H, C, | 77 | 77 | 14 | No |
| | | | (small cell) | (NE diff) | | | | | | |
| WA58 | CRPC | 76 | No ETS | NA | 82 | H, C, X | 141 | 141 | 39 | No |
| WA59 | CRPC | 59 | No ETS | NA | 1,678 | H, C, X | 94 | 94 | 12 | No |
| WA60 | CRPC | 62 | ERG⁺ | NA | 658 | H, C | 129 | 129 | 32 | No |

¹Localized prostate cancer (PC) or castrate resistant metastatic PC (CRPC).
² Age at diagnosis (PC) or death (CRPC).
³Rearrangements in ETS or RAF family genes or outlier expression of SPINK1.
⁴Gleason score of profiled prostatectomy specimen for PC. CRPCs with neuroendocrine (NE) differentiation are noted.
⁵Serum PSA at time of prostatectomy (PC) or death (CRPC).
⁶ P: prostatectomy; R: radiation; H: hormone therapy; C: chemotherapy; X: palliative radiation.
⁷Survival (in months) after diagnosis, first hormone therapy, and first chemotherapy.

TABLE 2

| | |
|---|---|
| METASTATIC (N = 50) | WA3 |

| | Average | Tumor | Normal |
|---|---|---|---|
| Exon Capture Kit | | Agilent 50 Mb | Agilent 50 Mb |
| Bases in target region | 50,555,052 | 51,712,500 | 51,712,500 |
| Bases in annotated | 31,910,087 | 32,295,535 | 32,295,535 |
| target region¹ | | | |
| Reads sequenced (after | 181,587,233 | 196,327,910 | 115,874,974 |
| quality filtering) | | | |
| Bases sequenced (after | 14,163,804,167 | 15,313,576,980 | 9,038,247,972 |
| quality filtering) | | | |
| Bases mapped to | 11,633,171,494 | 14,796,218,736 | 8,457,084,090 |
| genome | | | |
| Bases mapped to target | 6,032,795,932 | 5,693,889,174 | 3,697,877,285 |
| region | | | |
| Average number of | 119.33 | 110.11 | 71.51 |
| reads per targeted base | | | |
| Covered territory in the | 44,152,934 | 45,243,500 | |
| targeted region² | | | |
| % of targeted region | 89.12 | 87.49 | |
| that is covered² | | | |
| Annotated covered | 28,804,181 | 29,154,895 | |
| territory in the targeted | | | |
| region^{1,2} | | | |
| % of annotated targeted | 92.11 | 90.28 | |
| region that is covered^{1,2} | | | |
| Known SNPs identified | 35,228 | 35,255 | |
| in the targeted region³ | | | |
| Known SNPs identified | 18,690 | 18,384 | |
| in the annotated targeted | | | |
| region^{1,3} | | | |
| Somatic mutations | 57.67 | 38 | |
| identified in the | | | |
| annotated targeted | | | |
| region^{1,4} | | | |
| Mutation rate per Mb of | 2.00 | 1.30 | |
| annotated covered | | | |
| targeted territory^{1,2,4} | | | |

| | |
|---|---|
| WA7 | WA10 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 239,307,388 | 136,585,336 | 210,947,030 | 146,604,484 |
| quality filtering) | | | | |
| Bases sequenced (after | 18,665,976,264 | 10,653,656,208 | 16,453,868,340 | 11,435,149,752 |
| quality filtering) | | | | |
| Bases mapped to genome | 10,739,425,398 | 11,006,378,916 | 13,269,758,034 | 9,318,902,190 |
| Bases mapped to target | 7,517,100,745 | 4,618,700,263 | 6,468,234,052 | 4,765,571,452 |
| region | | | | |
| Average number of reads | 145.36 | 89.31 | 125.08 | 92.16 |
| per targeted base | | | | |
| Covered territory in the | 46,388,930 | | 46,306,761 | |
| targeted region² | | | | |
| % of targeted region that | 89.71 | | 89.55 | |
| is covered² | | | | |
| Annotated covered | 29,811,873 | | 29,786,595 | |
| territory in the targeted | | | | |
| region^{1,2} | | | | |
| % of annotated targeted | 92.31 | | 92.23 | |
| region that is covered^{1,2} | | | | |
| Known SNPs identified | 44,481 | | 36,987 | |
| in the targeted region³ | | | | |
| Known SNPs identified | 23,071 | | 19,167 | |
| in the annotated targeted | | | | |
| region^{1,3} | | | | |
| Somatic mutations | 39 | | 71 | |
| identified in the | | | | |
| annotated targeted | | | | |
| region^{1,4} | | | | |
| Mutation rate per Mb of | 1.31 | | 2.38 | |
| annotated covered | | | | |
| targeted territory^{1,2,4} | | | | |

| | |
|---|---|
| WA11 | WA12 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 233,284,100 | 248,534,864 | 279,310,600 | 119,730,942 |
| quality filtering) | | | | |
| Bases sequenced (after | 18,196,159,800 | 19,385,719,392 | 21,786,226,800 | 9,339,013,476 |
| quality filtering) | | | | |
| Bases mapped to genome | 14,996,964,138 | 15,948,268,284 | 17,817,073,170 | 7,663,101,732 |
| Bases mapped to target | 8,254,469,923 | 8,373,713,648 | 9,500,446,994 | 4,101,367,860 |
| region | | | | |
| Average number of reads | 159.62 | 161.93 | 183.72 | 79.31 |
| per targeted base | | | | |
| Covered territory in the | 46,436,738 | | 47,150,725 | |
| targeted region² | | | | |
| % of targeted region that | 89.80 | | 91.18 | |
| is covered² | | | | |
| Annotated covered | 29,831,709 | | 30,337,295 | |
| territory in the targeted | | | | |
| region^{1,2} | | | | |
| % of annotated targeted | 92.37 | | 93.94 | |
| region that is covered^{1,2} | | | | |
| Known SNPs identified | 37,210 | | 37,680 | |
| in the targeted region³ | | | | |
| Known SNPs identified | 19,249 | | 19,552 | |
| in the annotated targeted | | | | |
| region^{1,3} | | | | |
| Somatic mutations | 58 | | 79 | |
| identified in the | | | | |
| annotated targeted | | | | |
| region^{1,4} | | | | |
| Mutation rate per Mb of | 1.94 | | 2.60 | |
| annotated covered | | | | |
| targeted territory^{1,2,4} | | | | |

| | |
|---|---|
| WA13 | WA14 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 238,860,554 | 143,801,289 | 193,263,826 | 103,505,538 |
| quality filtering) | | | | |
| Bases sequenced (after | 18,631,123,212 | 11,216,500,542 | 15,074,578,428 | 8,073,431,964 |
| quality filtering) | | | | |
| Bases mapped to genome | 15,807,563,148 | 9,472,814,442 | 12,800,110,674 | 6,933,708,210 |
| Bases mapped to target | 8,056,848,680 | 5,271,824,997 | 6,335,614,001 | 3,655,411,669 |
| region | | | | |
| Average number of reads | 155.80 | 101.94 | 122.52 | 70.69 |
| per targeted base | | | | |
| Covered territory in the | 46,756,348 | | 46,079,248 | |
| targeted region² | | | | |
| % of targeted region that | 90.42 | | 89.11 | |
| is covered² | | | | |
| Annotated covered | 30,169,073 | | 29,796,988 | |
| territory in the targeted | | | | |
| region^{1,2} | | | | |
| % of annotated targeted | 93.42 | | 92.26 | |
| region that is covered^{1,2} | | | | |
| Known SNPs identified | 37,047 | | 37,970 | |
| in the targeted region³ | | | | |
| Known SNPs identified | 19,137 | | 19,995 | |
| in the annotated targeted | | | | |
| region^{1,3} | | | | |
| Somatic mutations | 40 | | 43 | |
| identified in the | | | | |
| annotated targeted | | | | |
| region^{1,4} | | | | |
| Mutation rate per Mb of | 1.33 | | 1.44 | |
| annotated covered | | | | |
| targeted territory^{1,2,4} | | | | |

| | |
|---|---|
| WA15 | WA16 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 221,483,758 | 175,582,958 | 219,717,294 | 140,967,186 |
| quality filtering) | | | | |
| Bases sequenced (after | 17,275,733,124 | 13,695,470,724 | 17,137,948,932 | 10,995,440,508 |
| quality filtering) | | | | |
| Bases mapped to genome | 14,173,907,436 | 11,141,845,650 | 13,616,192,538 | 8,712,997,800 |
| Bases mapped to target | 7,539,957,311 | 5,958,714,058 | 7,322,121,067 | 4,341,585,953 |
| region | | | | |
| Average number of reads | 145.81 | 115.23 | 141.59 | 83.96 |
| per targeted base | | | | |
| Covered territory in the | 47,139,093 | | 46,454,157 | |
| targeted region² | | | | |
| % of targeted region that | 91.16 | | 89.83 | |
| is covered² | | | | |
| Annotated covered | 30,291,542 | | 29,867,429 | |
| territory in the targeted | | | | |
| region^{1,2} | | | | |
| % of annotated targeted | 93.79 | | 92.48 | |
| region that is covered^{1,2} | | | | |
| Known SNPs identified | 37,783 | | 37,276 | |
| in the targeted region³ | | | | |
| Known SNPs identified | 19,555 | | 19,384 | |
| in the annotated targeted | | | | |
| region^{1,3} | | | | |
| Somatic mutations | 38 | | 731 | |
| identified in the | | | | |
| annotated targeted | | | | |
| region^{1,4} | | | | |
| Mutation rate per Mb of | 1.25 | | 24.47 | |
| annotated covered | | | | |
| targeted territory^{1,2,4} | | | | |

| | |
|---|---|
| WA17 | WA18 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 275,991,394 | 122,411,994 | 218,889,962 | 136,973,704 |
| quality filtering) | | | | |
| Bases sequenced (after | 21,527,328,732 | 9,548,135,532 | 17,073,417,036 | 10,683,948,912 |
| quality filtering) | | | | |
| Bases mapped to genome | 17,632,767,360 | 7,878,338,520 | 13,584,124,086 | 8,603,200,710 |
| Bases mapped to target | 9,474,257,157 | 4,476,084,331 | 7,332,432,992 | 4,396,258,502 |
| region | | | | |
| Average number of reads | 183.21 | 86.56 | 141.79 | 85.01 |
| per targeted base | | | | |
| Covered territory in the | 47,323,441 | | 46,392,775 | |
| targeted region² | | | | |
| % of targeted region that | 91.51 | | 89.71 | |
| is covered² | | | | |
| Annotated covered | 30,440,450 | | 29,852,343 | |
| territory in the targeted | | | | |
| region^{1,2} | | | | |
| % of annotated targeted | 94.26 | | 92.43 | |
| region that is covered^{1,2} | | | | |
| Known SNPs identified | 38,556 | | 36,990 | |
| in the targeted region³ | | | | |
| Known SNPs identified | 19,859 | | 19,279 | |
| in the annotated targeted | | | | |
| region^{1,3} | | | | |
| Somatic mutations | 36 | | 61 | |
| identified in the | | | | |
| annotated targeted | | | | |
| region^{1,4} | | | | |
| Mutation rate per Mb of | 1.18 | | 2.04 | |
| annotated covered | | | | |
| targeted territory^{1,2,4} | | | | |

| | |
|---|---|
| WA19 | WA20 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 180,622,432 | 132,935,604 | 249,457,368 | 140,335,034 |
| quality filtering) | | | | |
| Bases sequenced (after | 14,088,549,696 | 10,368,977,112 | 19,457,674,704 | 10,946,132,652 |
| quality filtering) | | | | |
| Bases mapped to genome | 12,021,462,024 | 8,787,667,356 | 16,340,526,774 | 9,177,739,116 |
| Bases mapped to target | 6,421,861,535 | 4,280,487,926 | 8,544,674,192 | 4,874,833,303 |
| region | | | | |
| Average number of reads | 124.18 | 82.77 | 165.23 | 94.27 |
| per targeted base | | | | |
| Covered territory in the | 46,716,014 | | 47,109,052 | |
| targeted region² | | | | |
| % of targeted region that | 90.34 | | 91.10 | |
| is covered² | | | | |
| Annotated covered | 30,086,276 | | 30,269,129 | |
| territory in the targeted | | | | |
| region^{1,2} | | | | |
| % of annotated targeted | 93.16 | | 93.73 | |
| region that is covered^{1,2} | | | | |
| Known SNPs identified | 37,686 | | 38,583 | |
| in the targeted region³ | | | | |
| Known SNPs identified | 19,580 | | 19,982 | |
| in the annotated targeted | | | | |
| region^{1,3} | | | | |
| Somatic mutations | 58 | | 32 | |
| identified in the | | | | |
| annotated targeted | | | | |
| region^{1,4} | | | | |
| Mutation rate per Mb of | 1.93 | | 1.06 | |
| annotated covered | | | | |
| targeted territory^{1,2,4} | | | | |

| | |
|---|---|
| WA22 | WA23 |

| | Tumor | Normal | Tumor | Normal |
|---|---|---|---|---|
| Exon Capture Kit | Roche NimbleGen | Roche NimbleGen | Agilent 50 Mb | Agilent 50 Mb |
| Bases in target region | 44,214,714 | 44,214,714 | 51,712,500 | 51,712,500 |
| Bases in annotated target | 29,956,948 | 29,956,948 | 32,295,535 | 32,295,535 |
| region¹ | | | | |
| Reads sequenced (after | 164,144,678 | 147,843,477 | 215,925,114 | 149,021,036 |
| quality filtering) | | | | |
| Bases sequenced (after | 12,803,284,884 | 11,531,791,206 | 16,842,158,892 | 11,623,640,808 |
| quality filtering) | | | | |
| Bases mapped to genome | 10,578,523,644 | 9,607,563,342 | 14,168,818,716 | 9,822,159,282 |
| Bases mapped to target | 4,913,576,797 | 4,481,180,101 | 7,433,109,108 | 5,324,090,988 |
| region | | | | |
| Average number of reads | 111.13 | 101.35 | 143.74 | 102.96 |
| per targeted base | | | | |
| Covered territory in the | 33,369,828 | | 47,140,717 | |
| targeted region² | | | | |
| % of targeted region that | 75.47 | | 91.16 | |
| is covered² | | | | |
| Annotated covered | 22,407,149 | | 30,290,977 | |
| territory in the targeted | | | | |
| region^{1,2} | | | | |
| % of annotated targeted | 74.80 | | 93.79 | |
| region that is covered^{1,2} | | | | |
| Known SNPs identified | 23,953 | | 38,714 | |
| in the targeted region³ | | | | |
| Known SNPs identified | 13,768 | | 20,057 | |
| in the annotated targeted | | | | |
| region^{1,3} | | | | |
| Somatic mutations | 68 | | 54 | |
| identified in the | | | | |
| annotated targeted | | | | |
| region^{1,4} | | | | |
| Mutation rate per Mb of | 3.03 | | 1.78 | |
| annotated covered | | | | |
| targeted territory^{1,2,4} | | | | |

| | |
|---|---|
| WA24 | WA25 |

| | Tumor | Normal | Tumor | Normal |
|---|---|---|---|---|
| Exon Capture Kit | Roche NimbleGen | Roche NimbleGen | Agilent 50 Mb | Agilent 50 Mb |
| Bases in target region | 44,214,714 | 44,214,714 | 51,712,500 | 51,712,500 |
| Bases in annotated target | 29,956,948 | 29,956,948 | 32,295,535 | 32,295,535 |
| region¹ | | | | |
| Reads sequenced (after | 140,056,954 | 175,780,311 | 225,473,042 | 133,911,886 |
| quality filtering) | | | | |
| Bases sequenced (after | 10,924,442,412 | 13,710,864,258 | 17,586,897,276 | 10,445,127,108 |
| quality filtering) | | | | |
| Bases mapped to genome | 8,922,365,946 | 11,183,062,488 | 14,240,008,536 | 8,488,906,062 |
| Bases mapped to target | 4,191,611,535 | 5,082,004,547 | 7,864,851,212 | 4,227,592,973 |
| region | | | | |
| Average number of reads | 94.80 | 114.94 | 152.09 | 81.75 |
| per targeted base | | | | |
| Covered territory in the | 33,192,924 | | 46,650,920 | |
| targeted region² | | | | |
| % of targeted region that | 75.07 | | 90.21 | |
| is covered² | | | | |
| Annotated covered | 22,282,421 | | 29,966,443 | |
| territory in the targeted | | | | |
| region^{1,2} | | | | |
| % of annotated targeted | 74.38 | | 92.79 | |
| region that is covered^{1,2} | | | | |
| Known SNPs identified | 23,132 | | 38,124 | |
| in the targeted region³ | | | | |
| Known SNPs identified | 13,281 | | 19,749 | |
| in the annotated targeted | | | | |
| region^{1,3} | | | | |
| Somatic mutations | 27 | | 45 | |
| identified in the | | | | |
| annotated targeted | | | | |
| region^{1,4} | | | | |
| Mutation rate per Mb of | 1.21 | | 1.50 | |
| annotated covered | | | | |
| targeted territory^{1,2,4} | | | | |

| | |
|---|---|
| WA26 | WA27 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 237,044,864 | 113,717,814 | 200,211,408 | 159,197,108 |
| quality filtering) | | | | |
| Bases sequenced (after | 18,489,499,392 | 8,869,989,492 | 15,616,489,824 | 12,417,374,424 |
| quality filtering) | | | | |
| Bases mapped to genome | 13,873,708,524 | 6,604,724,724 | 12,141,353,094 | 9,672,386,724 |
| Bases mapped to target | 7,126,827,519 | 3,375,643,202 | 6,716,474,825 | 5,258,972,291 |
| region | | | | |
| Average number of reads | 137.82 | 65.28 | 129.88 | 101.70 |
| per targeted base | | | | |
| Covered territory in the | 46,447,648 | | 47,087,793 | |
| targeted region² | | | | |
| % of targeted region that | 89.82 | | 91.06 | |
| is covered² | | | | |
| Annotated covered | 29,886,116 | | 30,281,811 | |
| territory in the targeted | | | | |
| region^{1,2} | | | | |
| % of annotated targeted | 92.54 | | 93.76 | |
| region that is covered^{1,2} | | | | |
| Known SNPs identified | 37,284 | | 38,193 | |
| in the targeted region³ | | | | |
| Known SNPs identified | 19,316 | | 19,856 | |
| in the annotated targeted | | | | |
| region^{1,3} | | | | |
| Somatic mutations | 89 | | 64 | |
| identified in the | | | | |
| annotated targeted | | | | |
| region^{1,4} | | | | |
| Mutation rate per Mb of | 2.98 | | 2.11 | |
| annotated covered | | | | |
| targeted territory^{1,2,4} | | | | |

| | |
|---|---|
| WA28 | WA29 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 208,738,196 | 136,249,250 | 210,895,022 | 135,023,442 |
| quality filtering) | | | | |
| Bases sequenced (after | 16,281,579,288 | 10,627,441,500 | 16,449,811,716 | 10,531,828,476 |
| quality filtering) | | | | |
| Bases mapped to genome | 13,721,689,722 | 8,845,390,944 | 13,659,676,602 | 8,749,443,378 |
| Bases mapped to target | 6,749,600,357 | 4,742,632,810 | 7,031,016,549 | 4,695,773,907 |
| region | | | | |
| Average number of reads | 130.52 | 91.71 | 135.96 | 90.81 |
| per targeted base | | | | |
| Covered territory in the | 46,602,558 | | 47,088,567 | |
| targeted region² | | | | |
| % of targeted region that | 90.12 | | 91.06 | |
| is covered² | | | | |
| Annotated covered | 29,960,377 | | 30,273,332 | |
| territory in the targeted | | | | |
| region^{1,2} | | | | |
| % of annotated targeted | 92.77 | | 93.74 | |
| region that is covered^{1,2} | | | | |
| Known SNPs identified | 37,391 | | 38,290 | |
| in the targeted region³ | | | | |
| Known SNPs identified | 19,490 | | 19,841 | |
| in the annotated targeted | | | | |
| region^{1,3} | | | | |
| Somatic mutations | 43 | | 37 | |
| identified in the | | | | |
| annotated targeted | | | | |
| region^{1,4} | | | | |
| Mutation rate per Mb of | 1.44 | | 1.22 | |
| annotated covered | | | | |
| targeted territory^{1,2,4} | | | | |

| | |
|---|---|
| WA30 | WA31 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 362,456,476 | 333,905,968 | 245,839,028 | 138,055,430 |
| quality filtering) | | | | |
| Bases sequenced (after | 28,271,605,128 | 26,044,665,504 | 19,175,444,184 | 10,768,323,540 |
| quality filtering) | | | | |
| Bases mapped to genome | 22,632,115,350 | 20,272,349,448 | 16,247,973,768 | 9,059,132,082 |
| Bases mapped to target | 11,707,807,344 | 10,630,403,255 | 8,461,961,403 | 4,688,761,061 |
| region | | | | |
| Average number of reads | 226.40 | 205.57 | 163.63 | 90.67 |
| per targeted base | | | | |
| Covered territory in the | 38,460,923 | | 47,002,587 | |
| targeted region² | | | | |
| % of targeted region that | 74.37 | | 90.89 | |
| is covered² | | | | |
| Annotated covered | 26,270,453 | | 30,218,730 | |
| territory in the targeted | | | | |
| region^{1,2} | | | | |
| % of annotated targeted | 81.34 | | 93.57 | |
| region that is covered^{1,2} | | | | |
| Known SNPs identified | 30,029 | | 37,349 | |
| in the targeted region³ | | | | |
| Known SNPs identified | 17,025 | | 19,449 | |
| in the annotated targeted | | | | |
| region^{1,3} | | | | |
| Somatic mutations | 52 | | 51 | |
| identified in the | | | | |
| annotated targeted | | | | |
| region^{1,4} | | | | |
| Mutation rate per Mb of | 1.98 | | 1.69 | |
| annotated covered | | | | |
| targeted territory^{1,2,4} | | | | |

| | |
|---|---|
| WA32 | WA33 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 274,193,435 | 295,401,842 | 225,342,444 | 155,309,776 |
| quality filtering) | | | | |
| Bases sequenced (after | 21,387,087,930 | 23,041,343,676 | 17,576,710,632 | 12,114,162,528 |
| quality filtering) | | | | |
| Bases mapped to genome | 17,195,811,360 | 18,741,163,272 | 14,639,993,550 | 10,244,778,960 |
| Bases mapped to target | 10,023,348,189 | 10,428,490,058 | 7,494,138,817 | 5,361,947,208 |
| region | | | | |
| Average number of reads | 193.83 | 201.66 | 144.92 | 103.69 |
| per targeted base | | | | |
| Covered territory in the | 38,492,279 | | 46,985,498 | |
| targeted region² | | | | |
| % of targeted region that | 74.44 | | 90.86 | |
| is covered² | | | | |
| Annotated covered | 26,430,591 | | 30,192,707 | |
| territory in the targeted | | | | |
| region^{1,2} | | | | |
| % of annotated targeted | 81.84 | | 93.49 | |
| region that is covered^{1,2} | | | | |
| Known SNPs identified | 28,650 | | 37,890 | |
| in the targeted region³ | | | | |
| Known SNPs identified | 16,282 | | 19,543 | |
| in the annotated targeted | | | | |
| region^{1,3} | | | | |
| Somatic mutations | 40 | | 79 | |
| identified in the | | | | |
| annotated targeted | | | | |
| region^{1,4} | | | | |
| Mutation rate per Mb of | 1.51 | | 2.62 | |
| annotated covered | | | | |
| targeted territory^{1,2,4} | | | | |

| | |
|---|---|
| WA35 | WA37 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 170,916,320 | 157,827,174 | 221,570,304 | 119,172,764 |
| quality filtering) | | | | |
| Bases sequenced (after | 13,331,472,960 | 12,310,519,572 | 17,282,483,712 | 9,295,475,592 |
| quality filtering) | | | | |
| Bases mapped to genome | 10,558,678,026 | 9,722,413,350 | 13,671,196,422 | 7,439,092,050 |
| Bases mapped to target | 5,238,994,222 | 4,443,761,328 | 7,103,859,216 | 3,719,957,273 |
| region | | | | |
| Average number of reads | 101.31 | 85.93 | 137.37 | 71.94 |
| per targeted base | | | | |
| Covered territory in the | 43,296,626 | | 46,452,271 | |
| targeted region² | | | | |
| % of targeted region that | 83.73 | | 89.83 | |
| is covered² | | | | |
| Annotated covered | 28,057,374 | | 29,862,803 | |
| territory in the targeted | | | | |
| region^{1,2} | | | | |
| % of annotated targeted | 86.88 | | 92.47 | |
| region that is covered^{1,2} | | | | |
| Known SNPs identified | 39,730 | | 37,217 | |
| in the targeted region³ | | | | |
| Known SNPs identified | 20,696 | | 19,358 | |
| in the annotated targeted | | | | |
| region^{1,3} | | | | |
| Somatic mutations | 91 | | 100 | |
| identified in the | | | | |
| annotated targeted | | | | |
| region^{1,4} | | | | |
| Mutation rate per Mb of | 3.24 | | 3.35 | |
| annotated covered | | | | |
| targeted territory^{1,2,4} | | | | |

| | |
|---|---|
| WA38 | WA39 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 219,925,406 | 114,235,678 | 188,748,634 | 136,864,796 |
| quality filtering) | | | | |
| Bases sequenced (after | 17,154,181,668 | 8,910,382,884 | 14,722,393,452 | 10,675,454,088 |
| quality filtering) | | | | |
| Bases mapped to genome | 13,601,694,600 | 7,229,707,680 | 11,659,069,656 | 8,365,173,570 |
| Bases mapped to target | 7,219,562,550 | 3,698,690,294 | 6,499,968,073 | 4,190,668,406 |
| region | | | | |
| Average number of reads | 139.61 | 71.52 | 125.69 | 81.04 |
| per targeted base | | | | |
| Covered territory in the | 46,578,199 | | 46,529,438 | |
| targeted region² | | | | |
| % of targeted region that | 90.07 | | 89.98 | |
| is covered² | | | | |
| Annotated covered | 29,963,222 | | 29,949,409 | |
| territory in the targeted | | | | |
| region^{1,2} | | | | |
| % of annotated targeted | 92.78 | | 92.74 | |
| region that is covered^{1,2} | | | | |
| Known SNPs identified | 37,526 | | 37,194 | |
| in the targeted region³ | | | | |
| Known SNPs identified | 19,414 | | 19,413 | |
| in the annotated targeted | | | | |
| region^{1,3} | | | | |
| Somatic mutations | 41 | | 20 | |
| identified in the | | | | |
| annotated targeted | | | | |
| region^{1,4} | | | | |
| Mutation rate per Mb of | 1.37 | | 0.67 | |
| annotated covered | | | | |
| targeted territory^{1,2,4} | | | | |

| | |
|---|---|
| WA40 | WA41 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 292,367,050 | 289,615,025 | 218,259,636 | 132,631,232 |
| quality filtering) | | | | |
| Bases sequenced (after | 22,804,629,900 | 22,589,971,950 | 17,024,251,608 | 10,345,236,096 |
| quality filtering) | | | | |
| Bases mapped to genome | 18,718,111,542 | 18,478,856,994 | 14,011,614,864 | 8,474,155,716 |
| Bases mapped to target | 10,022,053,738 | 10,371,600,062 | 7,552,276,767 | 4,431,343,302 |
| region | | | | |
| Average number of reads | 193.80 | 200.56 | 146.04 | 85.69 |
| per targeted base | | | | |
| Covered territory in the | 37,098,145 | | 46,653,018 | |
| targeted region² | | | | |
| % of targeted region that | 71.74 | | 90.22 | |
| is covered² | | | | |
| Annotated covered | 25,943,852 | | 29,996,620 | |
| territory in the targeted | | | | |
| region^{1,2} | | | | |
| % of annotated targeted | 80.33 | | 92.88 | |
| region that is covered^{1,2} | | | | |
| Known SNPs identified | 26,720 | | 37,611 | |
| in the targeted region³ | | | | |
| Known SNPs identified | 15,234 | | 19,633 | |
| in the annotated targeted | | | | |
| region^{1,3} | | | | |
| Somatic mutations | 83 | | 38 | |
| identified in the | | | | |
| annotated targeted | | | | |
| region^{1,4} | | | | |
| Mutation rate per Mb of | 3.20 | | 1.27 | |
| annotated covered | | | | |
| targeted territory^{1,2,4} | | | | |

| | WA42 | | WA43 | | | |
|---|---|---|---|---|---|---|
| | Tumor | Normal | Tumor 43-27 | Tumor 43-44 | Tumor 43-71 | Normal |
| Exon Capture | Agilent 50 Mb | Agilent 50 Mb | Agilent 50 Mb | Agilent 50 Mb | Agilent 50 Mb | Agilent 50 Mb |
| Kit | | | | | | |
| Bases in target | 51,712,500 | 51,712,500 | 51,712,500 | 51,712,500 | 51,712,500 | 51,712,500 |
| region | | | | | | |
| Bases in | 32,295,535 | 32,295,535 | 32,295,535 | 32,295,535 | 32,295,535 | 32,295,535 |
| annotated | | | | | | |
| target region¹ | | | | | | |
| Reads | 169,227,800 | 160,543,858 | 106,207,750 | 119,846,711 | 115,921,897 | 109,694,911 |
| sequenced | | | | | | |
| (after quality | | | | | | |
| filtering) | | | | | | |
| Bases | 13,199,768,400 | 12,522,420,924 | 8,284,204,500 | 9,348,043,458 | 9,041,907,966 | 8,556,203,058 |
| sequenced | | | | | | |
| (after quality | | | | | | |
| filtering) | | | | | | |
| Bases mapped | 11,152,269,258 | 10,584,966,288 | 7,078,252,896 | 8,062,722,408 | 7,819,368,804 | 7,301,239,530 |
| to genome | | | | | | |
| Bases mapped | 5,328,029,502 | 5,238,213,050 | 3,474,444,817 | 4,302,838,713 | 3,702,454,417 | 3,538,639,763 |
| to target region | | | | | | |
| Average | 103.03 | 101.29 | 67.19 | 83.21 | 71.60 | 68.43 |
| number of | | | | | | |
| reads per | | | | | | |
| targeted base | | | | | | |
| Covered | 45,858,277 | | 44,422,313 | 45,354,116 | 43,442,267 | |
| territory in the | | | | | | |
| targeted region² | | | | | | |
| % of targeted | 88.68 | | 85.90 | 87.70 | 84.01 | |
| region that is | | | | | | |
| covered² | | | | | | |
| Annotated | 29,577,254 | | 28,929,421 | 29,400,424 | 28,188,971 | |
| covered | | | | | | |
| territory in the | | | | | | |
| targeted | | | | | | |
| region^{1,2} | | | | | | |
| % of annotated | 91.58 | | 89.58 | 91.04 | 87.28 | |
| targeted region | | | | | | |
| that is | | | | | | |
| covered^{1,2} | | | | | | |
| Known SNPs | 42,828 | | 34,341 | 35,298 | 34,037 | |
| identified in the | | | | | | |
| targeted region³ | | | | | | |
| Known SNPs | 22,378 | | 18,235 | 18,685 | 18,081 | |
| identified in the | | | | | | |
| annotated | | | | | | |
| targeted | | | | | | |
| region^{1,3} | | | | | | |
| Somatic | 56 | | 51 | 58 | 41 | |
| mutations | | | | | | |
| identified in the | | | | | | |
| annotated | | | | | | |
| targeted | | | | | | |
| region^{1,4} | | | | | | |
| Mutation rate | 1.89 | | 1.76 | 1.97 | 1.45 | |
| per Mb of | | | | | | |
| annotated | | | | | | |
| covered | | | | | | |
| targeted | | | | | | |
| territory^{1,2,4} | | | | | | |

| | |
|---|---|
| WA46 | WA47 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 174,132,908 | 174,753,667 | 294,431,643 | 249,599,519 |
| quality filtering) | | | | |
| Bases sequenced (after | 13,582,366,824 | 13,630,786,026 | 22,965,668,154 | 19,468,762,482 |
| quality filtering) | | | | |
| Bases mapped to genome | 11,159,069,376 | 11,418,269,070 | 18,591,449,448 | 15,707,581,110 |
| Bases mapped to target | 5,653,532,988 | 5,428,312,362 | 10,198,862,814 | 9,060,414,013 |
| region | | | | |
| Average number of reads | 109.33 | 104.97 | 197.22 | 175.21 |
| per targeted base | | | | |
| Covered territory in the | 46,191,694 | | 38,579,846 | |
| targeted region² | | | | |
| % of targeted region that | 89.32 | | 74.60 | |
| is covered² | | | | |
| Annotated covered | 29,727,542 | | 26,403,540 | |
| territory in the targeted | | | | |
| region^{1,2} | | | | |
| % of annotated targeted | 92.05 | | 81.76 | |
| region that is covered^{1,2} | | | | |
| Known SNPs identified | 36,911 | | 30,126 | |
| in the targeted region³ | | | | |
| Known SNPs identified | 19,221 | | 17,129 | |
| in the annotated targeted | | | | |
| region^{1,3} | | | | |
| Somatic mutations | 81 | | 23 | |
| identified in the | | | | |
| annotated targeted | | | | |
| region^{1,4} | | | | |
| Mutation rate per Mb of | 2.72 | | 0.87 | |
| annotated covered | | | | |
| targeted territory^{1,2,4} | | | | |

| | |
|---|---|
| WA48 | WA49 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 213,718,136 | 143,372,258 | 155,083,960 | 150,941,382 |
| quality filtering) | | | | |
| Bases sequenced (after | 16,670,014,608 | 11,183,036,124 | 12,096,548,880 | 11,773,427,796 |
| quality filtering) | | | | |
| Bases mapped to genome | 13,840,286,460 | 9,134,503,716 | 10,251,439,692 | 9,990,123,234 |
| Bases mapped to target | 7,650,670,489 | 5,008,362,651 | 5,091,480,149 | 5,142,727,707 |
| region | | | | |
| Average number of reads | 147.95 | 96.85 | 98.46 | 99.45 |
| per targeted base | | | | |
| Covered territory in the | 46,994,028 | | 45,914,567 | |
| targeted region² | | | | |
| % of targeted region that | 90.88 | | 88.79 | |
| is covered² | | | | |
| Annotated covered | 30,236,283 | | 29,629,435 | |
| territory in the targeted | | | | |
| region^{1,2} | | | | |
| % of annotated targeted | 93.62 | | 91.74 | |
| region that is covered^{1,2} | | | | |
| Known SNPs identified | 37,641 | | 36,474 | |
| in the targeted region³ | | | | |
| Known SNPs identified | 19,620 | | 19,182 | |
| in the annotated targeted | | | | |
| regionl^{1,3} | | | | |
| Somatic mutations | 238 | | 33 | |
| identified in the | | | | |
| annotated targeted | | | | |
| region^{1,4} | | | | |
| Mutation rate per Mb of | 7.87 | | 1.11 | |
| annotated covered | | | | |
| targeted territory^{1,2,4} | | | | |

| | |
|---|---|
| WA50 | WA51 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 151,428,570 | 146,431,894 | 165,709,454 | 170,866,524 |
| quality filtering) | | | | |
| Bases sequenced (after | 11,811,428,460 | 11,421,687,732 | 12,925,337,412 | 13,327,588,872 |
| quality filtering) | | | | |
| Bases mapped to genome | 10,163,517,936 | 9,712,458,132 | 10,739,425,398 | 11,006,378,916 |
| Bases mapped to target | 4,825,565,562 | 4,918,010,589 | 4,824,644,281 | 5,103,100,934 |
| region | | | | |
| Average number of reads | 93.32 | 95.10 | 93.30 | 98.68 |
| per targeted base | | | | |
| Covered territory in the | 45,420,832 | | 45,700,087 | |
| targeted region² | | | | |
| % of targeted region that | 87.83 | | 88.37 | |
| is covered² | | | | |
| Annotated covered | 29,312,001 | | 29,470,664 | |
| territory in the targeted | | | | |
| region^{1,2} | | | | |
| % of annotated targeted | 90.76 | | 91.25 | |
| region that is covered^{1,2} | | | | |
| Known SNPs identified | 36,319 | | 36,043 | |
| in the targeted region³ | | | | |
| Known SNPs identified | 18,913 | | 18,816 | |
| in the annotated targeted | | | | |
| region^{1,3} | | | | |
| Somatic mutations | 92 | | 44 | |
| identified in the | | | | |
| annotated targeted | | | | |
| region^{1,4} | | | | |
| Mutation rate per Mb of | 3.14 | | 1.49 | |
| annotated covered | | | | |
| targeted territory^{1,2,4} | | | | |

| | |
|---|---|
| WA52 | WA53 |

| | Tumor | Normal | Tumor | Normal |
|---|---|---|---|---|
| Exon Capture Kit | Agilent 50 Mb | Agilent 50 Mb | Agilent 38 Mb | Agilent 38 Mb |
| Bases in target region | 51,712,500 | 51,712,500 | 37,806,033 | 37,806,033 |
| Bases in annotated target | 32,295,535 | 32,295,535 | 27,558,940 | 27,558,940 |
| region¹ | | | | |
| Reads sequenced (after | 196,334,388 | 182,664,677 | 170,043,479 | 160,836,761 |
| quality filtering) | | | | |
| Bases sequenced (after | 15,314,082,264 | 14,247,844,806 | 13,263,391,362 | 12,545,267,358 |
| quality filtering) | | | | |
| Bases mapped to genome | 12,710,410,284 | 11,851,474,518 | 11,275,509,726 | 10,592,709,894 |
| Bases mapped to target | 6,105,297,939 | 5,394,831,142 | 6,813,696,982 | 6,593,161,035 |
| region | | | | |
| Average number of reads | 118.06 | 104.32 | 180.23 | 174.39 |
| per targeted base | | | | |
| Covered territory in the | 46,200,459 | | 34,586,560 | |
| targeted region² | | | | |
| % of targeted region that | 89.34 | | 91.48 | |
| is covered² | | | | |
| Annotated covered | 29,689,775 | | 25,372,198 | |
| territory in the targeted | | | | |
| region^{1,2} | | | | |
| % of annotated targeted | 91.93 | | 92.07 | |
| region that is covered^{1,2} | | | | |
| Known SNPs identified | 37,283 | | 24,053 | |
| in the targeted region³ | | | | |
| Known SNPs identified | 19,351 | | 15,494 | |
| in the annotated targeted | | | | |
| region^{1,3} | | | | |
| Somatic mutations | 30 | | 35 | |
| identified in the | | | | |
| annotated targeted | | | | |
| region^{1,4} | | | | |
| Mutation rate per Mb of | 1.01 | | 1.38 | |
| annotated covered | | | | |
| targeted territory^{1,2,4} | | | | |

| | |
|---|---|
| WA54 | WA55 |

| | Tumor | Normal | Tumor | Normal |
|---|---|---|---|---|
| Exon Capture Kit | Agilent 38 Mb | Agilent 38 Mb | Agilent 38 Mb | Agilent 38 Mb |
| Bases in target region | 37,806,033 | 37,806,033 | 37,806,033 | 37,806,033 |
| Bases in annotated target | 27,558,940 | 27,558,940 | 27,558,940 | 27,558,940 |
| region¹ | | | | |
| Reads sequenced (after | 109,465,569 | 168,886,512 | 169,683,500 | 168,001,511 |
| quality filtering) | | | | |
| Bases sequenced (after | 8,538,314,382 | 13,173,147,936 | 13,235,313,000 | 13,104,117,858 |
| quality filtering) | | | | |
| Bases mapped to genome | 7,274,785,830 | 11,227,983,078 | 11,190,529,662 | 11,095,714,656 |
| Bases mapped to target | 4,409,679,103 | 7,016,349,732 | 6,730,794,029 | 6,872,481,717 |
| region | | | | |
| Average number of reads | 116.64 | 185.59 | 178.03 | 181.78 |
| per targeted base | | | | |
| Covered territory in the | 33,542,786 | | 34,336,412 | |
| targeted region² | | | | |
| % of targeted region that | 88.72 | | 90.82 | |
| is covered² | | | | |
| Annotated covered | 24,700,463 | | 25,216,056 | |
| territory in the targeted | | | | |
| region^{1,2} | | | | |
| % of annotated targeted | 89.63 | | 91.50 | |
| region that is covered^{1,2} | | | | |
| Known SNPs identified | 23,669 | | 23,783 | |
| in the targeted region³ | | | | |
| Known SNPs identified | 15,358 | | 15,336 | |
| in the annotated targeted | | | | |
| region^{1,3} | | | | |
| Somatic mutations | 34 | | 39 | |
| identified in the | | | | |
| annotated targeted | | | | |
| region^{1,4} | | | | |
| Mutation rate per Mb of | 1.38 | | 1.55 | |
| annotated covered | | | | |
| targeted territory^{1,2,4} | | | | |

| | |
|---|---|
| WA56 | WA57 |

| | Tumor | Normal | Tumor | Normal |
|---|---|---|---|---|
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 171,138,470 | 173,359,773 | 172,761,810 | 169,816,928 |
| quality filtering) | | | | |
| Bases sequenced (after | 13,348,800,660 | 13,522,062,294 | 13,475,421,180 | 13,245,720,384 |
| quality filtering) | | | | |
| Bases mapped to genome | 10,979,986,056 | 11,245,543,686 | 10,834,401,240 | 10,482,745,260 |
| Bases mapped to target | 5,177,318,788 | 5,497,614,730 | 4,778,197,473 | 5,096,773,379 |
| region | | | | |
| Average number of reads | 100.12 | 106.31 | 92.40 | 98.56 |
| per targeted base | | | | |
| Covered territory in the | 45,471,629 | | 43,235,107 | |
| targeted region² | | | | |
| % of targeted region that | 87.93 | | 83.61 | |
| is covered² | | | | |
| Annotated covered | 29,301,791 | | 27,914,483 | |
| territory in the targeted | | | | |
| region^{1,2} | | | | |
| % of annotated targeted | 90.73 | | 86.43 | |
| region that is covered^{1,2} | | | | |
| Known SNPs identified | 35,731 | | 33,315 | |
| in the targeted region³ | | | | |
| Known SNPs identified | 18,559 | | 17,487 | |
| in the annotated targeted | | | | |
| region^{1,3} | | | | |
| Somatic mutations | 169 | | 95 | |
| identified in the | | | | |
| annotated targeted | | | | |
| region^{1,4} | | | | |
| Mutation rate per Mb of | 5.77 | | 3.40 | |
| annotated covered | | | | |
| targeted territory^{1,2,4} | | | | |

| | WA58 | | WA59 | | WA60 | |
|---|---|---|---|---|---|---|
| | Tumor | Normal | Tumor | Normal | Tumor | Normal |
| Exon Capture Kit | Agilent 50 Mb | Agilent 50 Mb | Agilent 50 Mb | Agilent 50 Mb | Agilent 50 Mb | Agilent 50 Mb |
| Bases in target | 51,712,500 | 51,712,500 | 51,712,500 | 51,712,500 | 51,712,500 | 51,712,500 |
| region | | | | | | |
| Bases in annotated | 32,295,535 | 32,295,535 | 32,295,535 | 32,295,535 | 32,295,535 | 32,295,535 |
| target region¹ | | | | | | |
| Reads sequenced | 169,574,948 | 116,037,988 | 159,528,926 | 167,669,729 | 166,908,169 | 163,743,302 |
| (after quality | | | | | | |
| filtering) | | | | | | |
| Bases sequenced | 13,226,845,944 | 9,050,963,064 | 12,443,256,228 | 13,078,238,862 | 13,018,837,182 | 12,771,977,556 |
| (after quality | | | | | | |
| filtering) | | | | | | |
| Bases mapped to | 10,913,779,890 | 7,516,810,392 | 10,058,917,362 | 10,418,808,036 | 10,453,483,014 | 10,272,887,664 |
| genome | | | | | | |
| Bases mapped to | 5,832,827,648 | 3,953,746,265 | 4,475,487,277 | 4,900,312,121 | 4,569,838,785 | 4,726,398,012 |
| target region | | | | | | |
| Average number | 112.79 | 76.46 | 86.55 | 94.76 | 88.37 | 91.40 |
| of reads per | | | | | | |
| targeted base | | | | | | |
| Covered territory | 46,280,287 | | 42,802,930 | | 42,685,763 | |
| in the targeted | | | | | | |
| region² | | | | | | |
| % of targeted | 89.50 | | 82.77 | | 82.54 | |
| region that is | | | | | | |
| covered² | | | | | | |
| Annotated covered | 29,809,726 | | 27,664,217 | | 27,734,826 | |
| territory in the | | | | | | |
| targeted region^{1,2} | | | | | | |
| % of annotated | 92.30 | | 85.66 | | 85.88 | |
| targeted region | | | | | | |
| that is covered^{1,2} | | | | | | |
| Known SNPs | 37,850 | | 33,009 | | 34,211 | |
| identified in the | | | | | | |
| targeted region³ | | | | | | |
| Known SNPs | 19,759 | | 17,363 | | 17,943 | |
| identified in the | | | | | | |
| annotated targeted | | | | | | |
| region^{1,3} | | | | | | |
| Somatic mutations | 34 | | 31 | | 27 | |
| identified in the | | | | | | |
| annotated targeted | | | | | | |
| region^{1,4} | | | | | | |
| Mutation rate per | 1.14 | | 1.12 | | 0.97 | |
| Mb of annotated | | | | | | |
| covered targeted | | | | | | |
| territory^{1,2,4} | | | | | | |

¹A base is defined as “annotated” if it lies within a coding region in a CCDS or RefSeq transcript.
²A base is defined as “covered” if there are at least 14 reads (after PCR duplicate removal) overlapping the position in the tumor and 8 reads (after PCR duplicate removal) overlapping the position in the matched normal (see Methods).
³SNPs reported in dbSNP132 are identified if they have >=6 reads after PCR duplicate removal.
⁴Hyper-mutated samples, WA16, excluded from the the average. Excluding outlier samples WA48 and WA56 results in a mutation rate of 1.79/Mb. The median mutation rate is 1.53/Mb.

TABLE 3

T8

| | LOCALIZED (N = 11) | Tumor | Normal |
|---|---|---|---|
| Exon Capture Kit | Average | Agilent 38 Mb | Agilent 38 Mb |
| Bases in target region | 47,919,827 | 37,806,033 | 37,806,033 |
| Bases in annotated target | 31,003,736 | 27,558,940 | 27,558,940 |
| region¹ | | | |
| Reads sequenced (after | 145,663,025 | 67,298,821 | 104,368,062 |
| quality filtering) | | | |
| Bases sequenced (after | 11,361,715,978 | 5,249,308,038 | 8,140,708,836 |
| quality filtering) | | | |
| Bases mapped to genome | 9,239,787,740 | 4,452,510,738 | 6,968,251,602 |
| Bases mapped to target | 4,892,971,105 | 2,653,923,796 | 4,387,506,969 |
| region | | | |
| Average number of reads | 102.11 | 70.20 | 116.05 |
| per targeted base | | | |
| Covered territory in the | 42,100,632 | 29,737,501 | |
| targeted region² | | | |
| % of targeted region that is | 87.86 | 78.66 | |
| covered² | | | |
| Annotated covered territory | 27,987,286 | 22,411,708 | |
| in the targeted region^{1,2} | | | |
| % of annotated targeted | 90.27 | 81.32 | |
| region that is covered^{1,2} | | | |
| Known SNPs identified in | 33,510 | 21,051 | |
| the targeted region³ | | | |
| Known SNPs identified in | 18,214 | 13,895 | |
| the annotated targeted | | | |
| region^{1,3} | | | |
| Somatic mutations | 26.00 | 13 | |
| identified in the annotated | | | |
| targeted region¹ | | | |
| Mutation rate per Mb of | 0.93 | 0.58 | |
| annotated covered targeted | | | |
| territory^{1,2} | | | |

| | T12 | | T32 | |
|---|---|---|---|---|
| | Tumor | Normal | Tumor | Normal |
| Exon Capture Kit | Agilent 38 Mb | Agilent 38 Mb | Agilent 38 Mb | Agilent 38 Mb |
| Bases in target region | 37,806,033 | 37,806,033 | 37,806,033 | 37,806,033 |
| Bases in annotated target | 27,558,940 | 27,558,940 | 27,558,940 | 27,558,940 |
| region¹ | | | | |
| Reads sequenced (after | 64,236,912 | 68,451,174 | 75,148,294 | 49,402,659 |
| quality filtering) | | | | |
| Bases sequenced (after | 5,010,479,136 | 5,339,191,572 | 5,861,566,932 | 3,853,407,402 |
| quality filtering) | | | | |
| Bases mapped to genome | 4,038,645,156 | 4,440,184,320 | 5,142,062,016 | 3,409,918,824 |
| Bases mapped to target | 2,454,089,532 | 2,573,026,486 | 3,129,239,992 | 2,170,950,930 |
| region | | | | |
| Average number of reads | 64.91 | 68.06 | 82.77 | 57.42 |
| per targeted base | | | | |
| Covered territory in the | 29,764,119 | | 30,797,606 | |
| targeted region² | | | | |
| % of targeted region that is | 78.73 | | 81.46 | |
| covered² | | | | |
| Annotated covered territory | 22,349,747 | | 22,981,184 | |
| in the targeted region^{1,2} | | | | |
| % of annotated targeted | 81.10 | | 83.39 | |
| region that is covered^{1,2} | | | | |
| Known SNPs identified in | 20,990 | | 22,142 | |
| the targeted region³ | | | | |
| Known SNPs identified in | 13,830 | | 14,496 | |
| the annotated targeted | | | | |
| region^{1,3} | | | | |
| Somatic mutations | 24 | | 15 | |
| identified in the annotated | | | | |
| targeted region¹ | | | | |
| Mutation rate per Mb of | 1.07 | | 0.65 | |
| annotated covered targeted | | | | |
| territory^{1,2} | | | | |
| | T90 | | T91 | |
| | Tumor | Normal | Tumor | Normal |
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 236,160,382 | 125,850,478 | 239,088,968 | 122,328,864 |
| quality filtering) | | | | |
| Bases sequenced (after | 18,420,509,796 | 9,816,337,284 | 18,648,939,504 | 9,541,651,392 |
| quality filtering) | | | | |
| Bases mapped to genome | 13,923,908,778 | 7,433,490,870 | 14,956,345,092 | 7,748,750,334 |
| Bases mapped to target | 6,696,430,566 | 3,666,868,136 | 7,944,570,246 | 4,151,631,700 |
| region | | | | |
| Average number of reads | 129.49 | 70.91 | 153.63 | 80.28 |
| per targeted base | | | | |
| Covered territory in the | 46,465,741 | | 46,690,454 | |
| targeted region² | | | | |
| % of targeted region that is | 89.85 | | 90.29 | |
| covered² | | | | |
| Annotated covered territory | 29,909,472 | | 30,089,264 | |
| in the targeted region^{1,2} | | | | |
| % of annotated targeted | 92.61 | | 93.17 | |
| region that is covered^{1,2} | | | | |
| Known SNPs identified in | 38,185 | | 37,989 | |
| the targeted region³ | | | | |
| Known SNPs identified in | 19,781 | | 19,676 | |
| the annotated targeted | | | | |
| region^{1,3} | | | | |
| Somatic mutations | 38 | | 16 | |
| identified in the annotated | | | | |
| targeted region¹ | | | | |
| Mutation rate per Mb of | 1.27 | | 0.53 | |
| annotated covered targeted | | | | |
| territory^{1,2} | | | | |
| | T92 | | T93 | |
| | Tumor | Normal | Tumor | Normal |
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 196,802,766 | 138,528,356 | 225,233,344 | 120,452,230 |
| quality filtering) | | | | |
| Bases sequenced (after | 15,350,615,748 | 10,805,211,768 | 17,568,200,832 | 9,395,273,940 |
| quality filtering) | | | | |
| Bases mapped to genome | 12,785,361,654 | 9,025,618,056 | 14,245,493,964 | 7,683,751,764 |
| Bases mapped to target | 6,662,201,077 | 4,798,433,405 | 7,706,427,342 | 4,082,767,741 |
| region | | | | |
| Average number of reads | 128.83 | 92.79 | 149.02 | 78.95 |
| per targeted base | | | | |
| Covered territory in the | 46,777,601 | | 46,567,986 | |
| targeted region² | | | | |
| % of targeted region that is | 90.46 | | 90.05 | |
| covered² | | | | |
| Annotated covered territory | 30,144,456 | | 30,008,208 | |
| in the targeted region^{1,2} | | | | |
| % of annotated targeted | 93.34 | | 92.92 | |
| region that is covered^{1,2} | | | | |
| Known SNPs identified in | 38,487 | | 38,227 | |
| the targeted region³ | | | | |
| Known SNPs identified in | 19,917 | | 19,842 | |
| the annotated targeted | | | | |
| region^{1,3} | | | | |
| Somatic mutations | 34 | | 23 | |
| identified in the annotated | | | | |
| targeted region¹ | | | | |
| Mutation rate per Mb of | 1.13 | | 0.77 | |
| annotated covered targeted | | | | |
| territory^{1,2} | | | | |
| | T94 | | T95 | |
| | Tumor | Normal | Tumor | Normal |
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 205,043,118 | 136,755,100 | 192,588,236 | 111,835,084 |
| quality filtering) | | | | |
| Bases sequenced (after | 15,993,363,204 | 10,666,897,800 | 15,021,882,408 | 8,723,136,552 |
| quality filtering) | | | | |
| Bases mapped to genome | 12,896,278,278 | 8,640,529,560 | 11,543,774,190 | 6,756,801,792 |
| Bases mapped to target | 6,474,238,915 | 4,471,606,462 | 6,000,365,386 | 3,669,117,395 |
| region | | | | |
| Average number of reads | 125.20 | 86.47 | 116.03 | 70.95 |
| per targeted base | | | | |
| Covered territory in the | 46,573,485 | | 46,185,465 | |
| targeted region² | | | | |
| % of targeted region that is | 90.06 | | 89.31 | |
| covered² | | | | |
| Annotated covered territory | 30,017,021 | | 29,767,407 | |
| in the targeted region^{1,2} | | | | |
| % of annotated targeted | 92.94 | | 92.17 | |
| region that is covered^{1,2} | | | | |
| Known SNPs identified in | 38,047 | | 37,311 | |
| the targeted region³ | | | | |
| Known SNPs identified in | 19,820 | | 19,413 | |
| the annotated targeted | | | | |
| region^{1,3} | | | | |
| Somatic mutations | 14 | | 31 | |
| identified in the annotated | | | | |
| targeted region¹ | | | | |
| Mutation rate per Mb of | 0.47 | | 1.04 | |
| annotated covered targeted | | | | |
| territory^{1,2} | | | | |
| | T96 | | T97 | |
| | Tumor | Normal | Tumor | Normal |
| Exon Capture Kit | Agilent 50 Mb | Agilent 50 Mb | Agilent 50 Mb | Agilent 50 Mb |
| Bases in target region | 51,712,500 | 51,712,500 | 51,712,500 | 51,712,500 |
| Bases in annotated target | 32,295,535 | 32,295,535 | 32,295,535 | 32,295,535 |
| region¹ | | | | |
| Reads sequenced (after | 196,706,498 | 153,572,920 | 231,693,830 | 143,040,462 |
| quality filtering) | | | | |
| Bases sequenced (after | 15,343,106,844 | 11,978,687,760 | 18,072,118,740 | 11,157,156,036 |
| quality filtering) | | | | |
| Bases mapped to genome | 12,825,145,944 | 10,109,514,168 | 14,940,334,344 | 9,308,658,840 |
| Bases mapped to target | 6,389,494,544 | 5,045,944,636 | 7,927,187,836 | 4,589,341,216 |
| region | | | | |
| Average number of reads | 123.56 | 97.58 | 153.29 | 88.75 |
| per targeted base | | | | |
| Covered territory in the | 46,661,825 | | 46,885,164 | |
| targeted region² | | | | |
| % of targeted region that is | 90.23 | | 90.67 | |
| covered² | | | | |
| Annotated covered territory | 30,051,964 | | 30,129,716 | |
| in the targeted region^{1,2} | | | | |
| % of annotated targeted | 93.05 | | 93.29 | |
| region that is covered^{1,2} | | | | |
| Known SNPs identified in | 37,710 | | 38,469 | |
| the targeted region³ | | | | |
| Known SNPs identified in | 19,775 | | 19,912 | |
| the annotated targeted | | | | |
| region^{1,3} | | | | |
| Somatic mutations | 41 | | 37 | |
| identified in the annotated | | | | |
| targeted region¹ | | | | |
| Mutation rate per Mb of | 1.36 | | 1.23 | |
| annotated covered targeted | | | | |
| territory^{1,2} | | | | |

¹A base is defined as “annotated” if it lies within a coding region in a CCDS or RefSeq transcript.
²A base is defined as “covered” if there are at least 14 reads (after PCR duplicate removal) overlapping the position in the tumor and 8 reads (after PCR duplicate removal) overlapping the position in the matched normal (see Methods).
³SNPs reported in dbSNP132 are identified if they have >=6 reads after PCR duplicate removal.

TABLE 4

| | | | | | Matched |
|---|---|---|---|---|---|
| Sample | | ETS/RAF | aCGH | GE | exome |
| Name | Sample Type | status¹ | platform | platform | sequence² |
| N1 | Benign | NA | 105k | 1x44k | No |
| N2 | Benign | NA | 105k | 1x44k | No |
| N4 | Benign | NA | 105k | 1x44k | No |
| N5 | Benign | NA | 105k | 1x44k | No |
| N6 | Benign | NA | 105k | 1x44k | No |
| N7 | Benign | NA | 105k | 1x44k | No |
| N8 | Benign | NA | 105k | 1x44k | No |
| N9 | Benign | NA | 105k | 1x44k | No |
| N10 | Benign | NA | 105k | 1x44k | No |
| N11 | Benign | NA | 105k | 1x44k | No |
| N12 | Benign | NA | 105k | 1x44k | No |
| N13 | Benign | NA | 105k | 1x44k | No |
| N14 | Benign | NA | 105k | 1x44k | No |
| N15 | Benign | NA | 105k | 1x44k | No |
| N16 | Benign | NA | 105k | 1x44k | No |
| N17 | Benign | NA | 105k | 1x44k | No |
| N18 | Benign | NA | 105k | 4x44k | No |
| N19 | Benign | NA | 105k | 4x44k | No |
| N20 | Benign | NA | 105k | 4x44k | No |
| N21 | Benign | NA | 105k | 4x44k | No |
| N22 | Benign | NA | 105k | 4x44k | No |
| N23 | Benign | NA | 105k | 4x44k | No |
| N24 | Benign | NA | 105k | 4x44k | No |
| N25 | Benign | NA | 105k | 4x44k | No |
| N26 | Benign | NA | 105k | 4x44k | No |
| N27 | Benign | NA | 105k | 4x44k | No |
| N28 | Benign | NA | 105k | 4x44k | No |
| N29 | Benign | NA | 105k | 4x44k | No |
| T1 | Localized PC | No ETS | 105k | 1x44k | No |
| T3 | Localized PC | ETV1⁺ | 105k | 1x44k | No |
| T5 | Localized PC | ERG⁺ | 105k | 1x44k | No |
| T6 | Localized PC | No ETS | 105k | 1x44k | No |
| T7 | Localized PC | No ETS | 105k | 1x44k | No |
| T8 | Localized PC | ERG⁺ | 105k | 1x44k | Yes |
| T9a | Localized PC | ETV5⁺ | 105k | 1x44k | No |
| T10 | Localized PC | ETV5⁺ | 105k | 1x44k | No |
| T11 | Localized PC | ERG⁺ | 105k | 1x44k | No |
| T12 | Localized PC | ERG⁺ | 105k | 1x44k | Yes |
| T17 | Localized PC | ERG⁺ | 105k | 4x44k | No |
| T19 | Localized PC | ERG⁺ | 105k | 4x44k | No |
| T20 | Localized PC | ETV1⁺ | 105k | 4x44k | No |
| T21 | Localized PC | No ETS | 105k | 4x44k | No |
| T24 | Localized PC | No ETS | 105k | 4x44k | No |
| T25 | Localized PC | No ETS | 105k | 4x44k | No |
| T26 | Localized PC | No ETS | 105k | 4x44k | No |
| T27 | Localized PC | No ETS | 105k | 4x44k | No |
| T29 | Localized PC | No ETS | 105k | 4x44k | No |
| T31 | Localized PC | No ETS | 105k | 4x44k | No |
| T32 | Localized PC | RAF1⁺ | 105k | 4x44k | Yes |
| T37 | Localized PC | No ETS | 105k | 4x44k | No |
| T39 | Localized PC | ERG⁺ | 105k | 4x44k | No |
| T40 | Localized PC | No ETS | 105k | 4x44k | No |
| T41 | Localized PC | ERG⁺ | 105k | 4x44k | No |
| T42 | Localized PC | No ETS | 105k | 4x44k | No |
| T43 | Localized PC | No ETS | 105k | 4x44k | No |
| T44 | Localized PC | ERG⁺ | 105k | 4x44k | No |
| T45 | Localized PC | SPINK1⁺ | 105k | 4x44k | No |
| T46 | Localized PC | ERG⁺ | 105k | 4x44k | No |
| T47 | Localized PC | SPINK1⁺ | 105k | 4x44k | No |
| T48 | Localized PC | ERG⁺ | 105k | 4x44k | No |
| T49 | Localized PC | No ETS | 105k | 4x44k | No |
| T50 | Localized PC | ERG⁺ | 105k | 4x44k | No |
| T51 | Localized PC | ERG⁺ | 105k | 4x44k | No |
| T52 | Localized PC | ETV1⁺ | 105k | 4x44k | No |
| T53 | Localized PC | No ETS | 105k | 4x44k | No |
| T54 | Localized PC | ERG⁺ | 105k | 4x44k | No |
| T55 | Localized PC | No ETS | 105k | 4x44k | No |
| T56 | Localized PC | No ETS | 105k | 4x44k | No |
| T57 | Localized PC | ETV1⁺ | 105k | 4x44k | No |
| T58 | Localized PC | SPINK1⁺ | 105k | 4x44k | No |
| T59 | Localized PC | ERG⁺ | 105k | 4x44k | No |
| T60 | Localized PC | ERG⁺ | 105k | 4x44k | No |
| T61 | Localized PC | No ETS | 105k | 4x44k | No |
| T62 | Localized PC | ERG⁺ | 105k | 4x44k | No |
| T63 | Localized PC | ERG⁺ | 105k | 4x44k | No |
| T64 | Localized PC | ERG⁺ | 105k | 4x44k | No |
| T65 | Localized PC | No ETS | 105k | 4x44k | No |
| T66 | Localized PC | No ETS | 105k | 4x44k | No |
| T67 | Localized PC | No ETS | 105k | 4x44k | No |
| T68 | Localized PC | ERG⁺ | 105k | 4x44k | No |
| T69 | Localized PC | ERG⁺ | 105k | 4x44k | No |
| T70 | Localized PC | ERG⁺ | 105k | 4x44k | No |
| T73 | Localized PC | ERG⁺ | 105k | 4x44k | No |
| T75 | Localized PC | ERG⁺ | 105k | 4x44k | No |
| T82 | Localized PC | ERG⁺ | 105k | 4x44k | No |
| T83 | Localized PC | ERG⁺ | 105k | 4x44k | No |
| T85 | Localized PC | ERG⁺ | 105k | 4x44k | No |
| WA2 | CRPC | ERG⁺ | 105k | 4x44k | No |
| WA3 | CRPC | No ETS | 105k | 1x44K | Yes |
| WA4 | CRPC | ERG⁺ | 105k | 1x44K | No |
| WA5 | CRPC | SPINK1⁺ | 105k | 1x44K | No |
| WA6 | CRPC | ERG⁺ | 105k | 4x44k | No |
| WA7 | CRPC | No ETS | 105k | 4x44k | Yes |
| WA10 | CRPC | No ETS | 105k | 4x44k | Yes |
| WA11 | CRPC | No ETS | 105k | 4x44k | Yes |
| WA13 | CRPC | ERG⁺ | 105k | 1x44K | Yes |
| WA14 | CRPC | No ETS | 105k | 4x44k | Yes |
| WA16 | CRPC | ERG⁺ | 105k | 1x44K | Yes |
| WA18 | CRPC | ERG⁺ | 105k | 4x44k | Yes |
| WA19 | CRPC | No ETS | 105k | 4x44k | Yes |
| WA20 | CRPC | No ETS | 105k | 1x44K | Yes |
| WA22 | CRPC | ERG⁺ | 105k | 1x44K | Yes |
| | | (small | | | |
| | | cell) | | | |
| WA23 | CRPC | ETV1⁺ | 105k | 4x44k | Yes |
| WA24 | CRPC | ERG⁺ | 105k | 1x44K | Yes |
| | | (small | | | |
| | | cell) | | | |
| WA25 | CRPC | No ETS | 105k | 4x44k | Yes |
| WA26 | CRPC | ETV1⁺ | 105k | 4x44k | Yes |
| WA28 | CRPC | ERG⁺ | 105k | 4x44k | Yes |
| WA29 | CRPC | No ETS | 105k | 4x44k | Yes |
| WA30 | CRPC | No ETS | 105k | 4x44k | Yes |
| WA31 | CRPC | ERG⁺ | 105k | 4x44k | Yes |
| WA32 | CRPC | No ETS | 105k | 4x44k | Yes |
| WA33 | CRPC | No ETS | 105k | 4x44k | Yes |
| WA35 | CRPC | No ETS | 105k | 4x44k | Yes |
| WA37 | CRPC | ERG⁺ | 105k | 4x44k | Yes |
| WA39 | CRPC | ERG⁺ | 105k | 4x44k | Yes |
| WA40 | CRPC | ERG⁺ | 105k | 4x44k | Yes |
| WA42 | CRPC | No ETS | 105k | 4x44k | Yes |
| WA46 | CRPC | No ETS | 105k | 4x44k | Yes |
| WA47 | CRPC | SPINK1⁺ | 105k | 4x44k | Yes |
| WA53 | CRPC | ERG⁺ | 244k | 4x44k | Yes |
| WA54 | CRPC | ERG⁺ | 244k | 4x44k | Yes |
| WA55 | CRPC | ERG⁺ | 244k | 4x44k | Yes |

¹Rearrangements in indicated ETS or RAF family genes or outlier expression of SPINK1.
²Matched samples also used for exome sequencing are indicated.

TABLE 5

| | Tissue | Androgen | |
|---|---|---|---|
| Cell Line | Source | Signaling | Notes |
| WPE1- | NA | Androgen | HPV infected benign prostatic |
| NB26 | | Sensitive | epithelial cells treated with N-methyl- |
| | | | N-nitrosourea |
| CWR22 | Localized | Androgen | |
| | | Sensitive | |
| 22RV1 | NA | Androgen | Derivative of CWR22 |
| | | Insensitive | |
| LNCaP | Metastasis | Androgen | |
| | | Sensitive | |
| C4-2B | NA | Androgen | Derivative of LNCaP |
| | | Insensitive | |
| VCaP | Metastasis | Androgen | |
| | | Sensitive | |
| LAPC-4 | Metastasis | Androgen | |
| | | Sensitive | |
| MDA- | Metastasis | Androgen | |
| Pca-2B | | Sensitive | |
| NCI-H660 | Metastasis | Androgen | Small cell carcinoma |
| | | Insensitive | |
| PC3 | Metastasis | Androgen | |
| | | Insensitive | |
| DU-145 | Metastasis | Androgen | |
| | | Insensitive | |

TABLE 6

| | | | | Amino | | |
|---|---|---|---|---|---|---|
| | Gene | Transcript | | acid | Mutation | Reads |
| Sample | Symbol¹ | Accession | Nucleotide (genomic)² | (protein) | type | (variant/total) |
| DU-145 | ABCC2 | CCDS7484.1 | g.chr10: 101581836insT | p.D1072fs | Indel | 10/24 |
| DU-145 | ACIN1 | CCDS9587.1 | g.chr14: 22608534delCTC | p.E808fp | Indel | 4/72 |
| DU-145 | ADCY9 | CCDS32382.1 | g.chr16: 3969187delG | p.A869fs | Indel | 4/7 |
| MDA- | AGAP3 | CCDS43681.1 | g.chr7: 150471365delGAG | p.E759fp | Indel | 6/18 |
| PCa-2B | | | | | | |
| DU-145 | AHNAK | CCDS31584.1 | g.chr11: 62054862delG | p.D1200fs | Indel | 5/12 |
| C4-2B | AKT2 | CCDS12552.1 | g.chr19: 45433066delCCT | p.K400fp | Indel | 9/50 |
| LAPC-4 | B4GALNT4 | CCDS7694.1 | g.chr11: 370927delG | p.G990fs | Indel | 10/32 |
| DU-145 | BCL2L11 | CCDS2089.1 | g.chr2: 111638201insA | p.A173fs | Indel | 6/17 |
| LAPC-4 | CNOT1 | CCDS10799.1 | g.chr16: 57178650delC | p.F128fs | Indel | 8/20 |
| CWR22 | ERBB2 | CCDS32642.1 | g.chr17: 35137305delC | p.L1130fs | Indel | 10/42 |
| LAPC-4 | FASN | CCDS11801.1 | g.chr17: 77636717delT | p.G1349fs | Indel | 4/85 |
| MDA- | FLNB | CCDS2885.1 | g.chr3: 58115694delT | p.G2257fs | Indel | 5/19 |
| PCa-2B | | | | | | |
| LAPC-4 | FOXA1 | CCDS9665.1 | g.chr14: 37130661delA | p.P358fs | Indel | 9/36 |
| DU-145 | FOXA1 | CCDS9665.1 | g.chr14: 37130720delC | p.A339fs | Indel | 9/26 |
| 22Rv1 | HOOK2 | CCDS42507.1 | g.chr19: 12739929delT | p.E370fs | Indel | 4/17 |
| CWR22 | HOOK2 | CCDS42507.1 | g.chr19: 12739929delT | p.E370fs | Indel | 4/23 |
| DU-145 | HPCAL1 | CCDS1671.1 | g.chr2: 10484347delC | p.S177fs | Indel | 7/77 |
| CWR22 | HSF1 | CCDS6419.1 | g.chr8: 145506521delC | p.R308fs | Indel | 4/28 |
| DU-145 | MAP7D1 | CCDS30673.1 | g.chr1: 36394649delC | p.P14fs | Indel | 6/36 |
| DU-145 | MBNL1 | CCDS3163.1 | g.chr3: 153615447delT | p.Y67fs | Indel | 8/75 |
| LAPC-4 | MKL2 | CCDS32391.1 | g.chr16: 14253807delC | p.R833fs | Indel | 9/24 |
| MDA- | MLL3 | CCDS5931.1 | g.chr7: 151473286delA | p.N4685fs | Indel | 4/8 |
| PCa-2B | | | | | | |
| DU-145 | MTMR11 | CCDS942.1 | g.chr1: 148167772delAAC | p.Q593fp | Indel | 9/25 |
| DU-145 | NR1D2 | CCDS33718.1 | g.chr3: 23984408delG | p.G477fs | Indel | 14/20 |
| 22Rv1 | NUFIP2 | CCDS32600.1 | g.chr17: 24638466delC | p.R223fs | Indel | 5/18 |
| CWR22 | OTUD7B | CCDS41389.1 | g.chr1: 148183560delG | p.P449fs | Indel | 9/17 |
| DU-145 | PFKP | CCDS7059.1 | g.chr10: 3148994delC | p.G466fs | Indel | 7/69 |
| LAPC-4 | PPP4R1 | CCDS42412.1 | g.chr18: 9539194delC | p.K895fs | Indel | 4/11 |
| LAPC-4 | SPHK2 | CCDS12727.1 | g.chr19: 53824463delC | p.P528fs | Indel | 4/6 |
| LAPC-4 | TRAF7 | CCDS10461.1 | g.chr16: 2155880insG | p.T27fs | Indel | 9/35 |
| MDA- | TRIP12 | CCDS33391.1 | g.chr2: 230432453delC | p.T59fs | Indel | 4/11 |
| PCa-2B | | | | | | |
| DU-145 | TUBGCP2 | CCDS7676.1 | g.chr10: 134943320delT | p.Q882fs | Indel | 9/41 |
| C4-2B | UBIAD1 | CCDS129.1 | g.chr1: 11268419delC | p.L220fs | Indel | 11/41 |

¹Only genes reported to be mutated in prostate tumor exome data were considered.

TABLE 7

| | | | | | | | | Exact | |
|---|---|---|---|---|---|---|---|---|---|
| | | | | | | | | nucleotide | |
| | | | | Amino | | Reads | Exome | change in | |
| | Gene | Transcript | Nucleotide | acid | Mutation | (variant/ | sequencing | exome | |
| Sample | Symbol | Accession | (genomic)¹ | (protein) | type | total) | samples | sequencing? | DBSNP? |
| C4-2B | AR | CCDS14387.1, | g.chrX: 66860277A > G | p.T878A, | Missense | 26/26 | WA42; WA13; | yes | dbsnp132-no |
| | | CCDS43965.1 | | p.T346A | | | WA32 | | |
| LNCaP | AR | CCDS14387.1, | g.chrX: 66860277A > G | p.T878A, | Missense | 104/104 | WA42; WA13; | yes | dbsnp132-no |
| | | CCDS43965.1 | | p.T346A | | | WA32 | | |
| MDA- | AR | CCDS14387.1, | g.chrX: 66860277A > G | p.T878A, | Missense | 16/16 | WA42; WA13; | yes | dbsnp132-no |
| PCa-2B | | CCDS43965.1 | | p.T346A | | | WA32 | | |
| MDA- | AR | CCDS14387.1, | g.chrX: 66848188T > A | p.L702H, | Missense | 75/75 | WA48 | yes | dbsnp132-no |
| PCa-2B | | CCDS43965.1 | | p.L170H | | | | | |
| 22Rv1 | AR | CCDS14387.1, | g.chrX: 66860268C > T | p.H875Y, | Missense | 7/7 | WA48; WA52 | yes | dbsnp132-no |
| | | CCDS43965.1 | | p.H343Y | | | | | |
| MDA- | LARP1B | CCDS3738.1, | g.chr4: 129218558C > T | p.R70C, | Missense | 5/8 | WA16 | yes | dbsnp132-no |
| PCa-2B | | CCDS47132.1, | | p.R70C, | | | | | |
| | | CCDS47133.1 | | p.R70C | | | | | |
| DU-145 | LGSN | CCDS4964.1 | g.chr6: 64048354G > T | p.A354E | Missense | 4/4 | T90 | no | dbsnp132-no |
| PC3 | MAST1 | CCDS32921.1 | g.chr19: 12815416G > A | p.V108I | Missense | 3/4 | WA16 | yes | dbsnp132-no |
| PC3 | S1PR3 | CCDS6680.1 | g.chr9: 90806410G > T | p.G159W | Missense | 4/4 | WA18 | no | rs56368313:G/ |
| | | | | | | | | | T:+:val = NO |
| LNCaP | STAG2 | CCDS14607.1, | g.chrX: 123012742C > T | p.R370W, | Missense | 34/34 | WA32 | no | dbsnp132-no |
| | | CCDS43990.1 | | p.R370W | | | | | |
| LAPC-4 | TRRAP | CCDS5659.1 | g.chr7: 98391959C > T | p.P2008L | Missense | 8/20 | WA16 | yes | dbsnp132-no |

TABLE 8

| | | | | Amino | |
|---|---|---|---|---|---|
| | Gene | Transcript | Nucleotide | acid | Mutation |
| Sample | Symbol | Accession | (genomic)¹ | (protein) | type |
| MDA- | ACTR10 | CCDS32090.1 | g.chr14: 57736765A > G | p.K26R | Missense |
| PCa-2B | | | | | |
| LAPC-4 | AKT1 | CCDS9994.1 | g.chr14: 104317596C > T | p.E17K | Missense |
| MDA- | APC | CCDS4107.1 | g.chr5: 112203550A > G | p.K1454E | Missense |
| PCa-2B | | | | | |
| LAPC-4 | BAP1 | CCDS2853.1 | g.chr3: 52415412C > T | p.R227H | Missense |
| C4-2B | BEND3 | CCDS34507.1 | g.chr6: 107497569C > T | p.D507N | Missense |
| CWR22 | BRAF | CCDS5863.1 | g.chr7: 140099614A > C | p.L597R | Missense |
| LNCaP | BSDC1 | CCDS363.1, | g.chr1: 32622186C > A | p.Q80H, p.Q80H | Missense |
| | | CCDS44103.1 | | | |
| VCaP | BSDC1 | CCDS363.1, | g.chr1: 32622186C > A | p.Q80H, p.Q80H | Missense |
| | | CCDS44103.1 | | | |
| CWR22 | BSDC1 | CCDS363.1, | g.chr1: 32622186C > A | p.Q80H, p.Q80H | Missense |
| | | CCDS44103.1 | | | |
| MDA- | CCDC123 | CCDS32987.1 | g.chr19: 38142646C > T | p.R102Q | Missense |
| PCa-2B | | | | | |
| 22Rv1 | CCDS33757.1, | CCDS33757.1, | g.chr3: 49697002A > G | p.W607R, | Missense |
| | MST1 | CCDS33757.2 | | p.W621R | |
| DU- | CDKN2A | CCDS6510.1 | g.chr9: 21961108C > A | p.D84Y | Missense |
| 145 | | | | | |
| DU- | CDKN2A | CCDS34998.1, | g.chr9: 21964775T > G | p.T18P, p.T18P | Missense |
| 145 | | CCDS6510.1 | | | |
| CWR22 | CHST2 | CCDS3129.1 | g.chr3: 144323462G > A | p.A372T | Missense |
| 22Rv1 | CHST2 | CCDS3129.1 | g.chr3: 144323462G > A | p.A372T | Missense |
| LAPC-4 | CNOT3 | CCDS12880.1 | g.chr19: 59338699G > A | p.E20K | Missense |
| DU- | CTNNB1 | CCDS2694.1 | g.chr3: 41241072T > G | p.V22G | Missense |
| 145 | | | | | |
| LNCaP | DDX50 | CCDS7283.1 | g.chr10: 70365827C > A | p.S527R | Missense |
| C4-2B | DNAJA2 | CCDS10726.1 | g.chr16: 45564914A > T | p.L24Q | Missense |
| 22Rv1 | DOK6 | CCDS32841.1 | g.chr18: 65576032G > A | p.A267T | Missense |
| LAPC-4 | EIF4ENIF1 | CCDS13898.1 | g.chr22: 30165993C > T | p.R944H | Missense |
| DU- | EP400 | CCDS31929.1 | g.chr12: 131082509A > C | p.H1937P | Missense |
| 145 | | | | | |
| CWR22 | EPHB4 | CCDS5706.1 | g.chr7: 100248764T > C | p.T587A | Missense |
| DU- | EXTL3 | CCDS6070.1 | g.chr8: 28631264T > C | p.L590P | Missense |
| 145 | | | | | |
| C4-2B | FKBP8 | CCDS32961.1 | g.chr19: 18511471C > G | p.V118L | Missense |
| LAPC-4 | GAS8 | CCDS10992.1 | g.chr16: 88631217G > A | p.R278H | Missense |
| DU- | GMPR2 | CCDS41935.1, | g.chr14: 23777661T > G | p.V295G, | Missense |
| 145 | | CCDS45087.1 | | p.V313G | |
| C4-2B | GNA11 | CCDS12103.1 | g.chr19: 3069941C > A | p.Q209K | Missense |
| LAPC-4 | GNAI2 | CCDS2813.1 | g.chr3: 50265496G > A | p.A114T | Missense |
| LAPC-4 | GPS2 | CCDS11100.1 | g.chr17: 7157157C > T | p.R272H | Missense |
| DU- | HERC2 | CCDS10021.1 | g.chr15: 26086511C > T | p.A3491T | Missense |
| 145 | | | | | |
| DU- | HIST1H2BD | CCDS4587.1 | g.chr6: 26266492C > A | p.S39* | Nonsense |
| 145 | | | | | |
| DU- | HRAS | CCDS7698.1, | g.chr11: 524282A > C | p.V14G, p.V14G | Missense |
| 145 | | CCDS7699.1 | | | |
| LNCaP | HSPA1L | CCDS34413.1 | g.chr6: 31886086G > T | p.A548D | Missense |
| LNCaP | HUS1 | CCDS34635.1 | g.chr7: 47984896A > G | p.L32P | Missense |
| DU- | KCNQ5 | CCDS4976.1 | g.chr6: 73843880G > A | p.R244H | Missense |
| 145 | | | | | |
| CWR22 | KIF3A | CCDS34235.1 | g.chr5: 132066159G > C | p.A556G | Missense |
| 22Rv1 | KIF3A | CCDS34235.1 | g.chr5: 132066159G > C | p.A556G | Missense |
| LNCaP | MAMDC4 | CCDS7010.1 | g.chr9: 138871944T > G | p.V780G | Missense |
| CWR22 | MEN1 | CCDS31600.1, | g.chr11: 64331625G > A | p.S253L, | Missense |
| | | CCDS8083.1 | | p.S258L | |
| 22Rv1 | MEN1 | CCDS31600.1, | g.chr11: 64331625G > A | p.S253L, | Missense |
| | | CCDS8083.1 | | p.S258L | |
| LAPC-4 | MTO1 | CCDS34485.1, | g.chr6: 74248614G > A | p.R464H, | Missense |
| | | CCDS47452.1, | | p.R504H, | |
| | | CCDS4979.1 | | p.R489H | |
| DU- | NAV3 | CCDS41815.1 | g.chr12: 76968851G > A | p.R770Q | Missense |
| 145 | | | | | |
| LNCaP | NEK9 | CCDS9839.1 | g.chr14: 74627811C > T | p.R786Q | Missense |
| C4-2B | NEK9 | CCDS9839.1 | g.chr14: 74627811C > T | p.R786Q | Missense |
| MDA- | NLE1 | CCDS11291.1, | g.chr17: 30487503G > T | p.Q319K, | Missense |
| PCa-2B | | CCDS45647.1 | | p.Q27K | |
| LAPC-4 | NPTN | CCDS10249.1, | g.chr15: 71666936C > T | p.A230T, | Missense |
| | | CCDS10250.1 | | p.A114T | |
| LNCaP | PSMD3 | CCDS11356.1 | g.chr17: 35405006G > T | p.G383V | Missense |
| C4-2B | PSMD3 | CCDS11356.1 | g.chr17: 35405006G > T | p.G383V | Missense |
| MDA- | PTEN | NM_000314 | g.chr10: 89682885G > A | p.R130Q | Missense |
| PCa-2B | | | | | |
| LNCaP | RAPGEF1 | CCDS48047.1, | g.chr9: 133449610T > A | p.K922M, | Missense |
| | | CCDS48048.1 | | p.K940M | |
| LAPC-4 | RASA1 | CCDS34200.1, | g.chr5: 86703757C > T | p.R589C, | Missense |
| | | CCDS47243.1 | | p.R412C | |
| C4-2B | SLCO4A1 | CCDS13501.1 | g.chr20: 60762294G > A | p.A325T | Missense |
| LAPC-4 | SMTN | CCDS13886.1, | g.chr22: 29817264C > G | p.R419G, | Missense |
| | | CCDS13887.1, | | p.R419G, | |
| | | CCDS13888.1 | | p.R419G | |
| DU- | STK11 | CCDS45896.1 | g.chr19: 1171442C > T | p.P179S | Missense |
| 145 | | | | | |
| LAPC-4 | TADA2A | CCDS11319.1 | g.chr17: 32904737G > A | p.R339Q | Missense |
| LNCaP | THNSL1 | CCDS7147.1 | g.chr10: 25353354A > G | p.Q399R | Missens |
| DU- | TMBIM4 | CCDS41805.1 | g.chr12: 64832397A > G | p.L78P | Missense |
| 145 | | | | | |
| LAPC-4 | TP53 | CCDS11118.1, | g.chr17: 7519131C > T | p.R175H, | Missense |
| | | CCDS45605.1, | | p.R175H, | |
| | | CCDS45606.1 | | p.R175H | |
| DU- | TP53 | CCDS11118.1, | g.chr17: 7517843C > A | p.V274F, | Missense |
| 145 | | CCDS45605.1, | | p.V274F, | |
| | | CCDS45606.1 | | p.V274F | |
| VCaP | TP53 | CCDS11118.1, | g.chr17: 7518264G > A | p.R248W, | Missense |
| | | CCDS45605.1, | | p.R248W, | |
| | | CCDS45606.1 | | p.R248W | |
| C4-2B | TP53 | CCDS11118.1, | g.chr17: 7518306A > G | p.Y234H, | Missense |
| | | CCDS45605.1, | | p.Y234H, | |
| | | CCDS45606.1 | | p.Y234H | |
| VCaP | TRAF4 | CCDS11243.1 | g.chr17: 24100652G > A | p.R448Q | Missense |
| C4-2B | TRIM16 | CCDS11171.1 | g.chr17: 15476641A > G | p.S308P | Missense |
| LNCaP | UBE2O | CCDS32742.1 | g.chr17: 71898726G > A | p.L1258F | Missense |
| CWR22 | ZDHHC11 | CCDS3857.1 | g.chr5: 886915G > T | p.A303D | Missense |
| LNCaP | ZDHHC11 | CCDS3857.1 | g.chr5: 886915G > T | p.A303D | Missense |
| NCI- | ZDHHC11 | CCDS3857.1 | g.chr5:886915G > T | p.A303D | Missense |
| H660 | | | | | |
| C4-2B | ZDHHC11 | CCDS3857.1 | g.chr5:886915G > T | p.A303D | Missense |

| | | | Exact | | |
|---|---|---|---|---|---|
| | | COSMIC | nucleotide | | |
| | | Amino | change | | |
| | Reads | acid | in | COSMIC | |
| Sample | (variant/total) | (protein) | COSMIC? | ID | DBSNP? |
| MDA- | 3/3 | p.K26K | no | NA | dbsnp132- |
| PCa-2B | | | | | no |
| LAPC-4 | 100/241 | p.E17K | yes | 33765 | dbsnp132- |
| | | | | | no |
| MDA- | 6/6 | p.K1454E | yes | 27993 | rs111866410:A/ |
| PCa-2B | | | | | G:+:val = |
| | | | | | NO |
| LAPC-4 | 8/24 | p.R227C | no | NA | dbsnp132- |
| | | | | | no |
| C4-2B | 3/7 | p.D507N | yes | 117464 | dbsnp132- |
| | | | | | no |
| CWR22 | 5/6 | p.L597R; | yes | 471 | dbsnp132- |
| | | p.L597Q; | | | no |
| | | p.L597L; | | | |
| | | p.L597V | | | |
| LNCaP | 3/5 | p.Q80* | no | NA | dbsnp132- |
| | | | | | no |
| VCaP | 5/9 | p.Q80* | no | NA | dbsnp132- |
| | | | | | no |
| CWR22 | 4/9 | p.Q80* | no | NA | dbsnp132- |
| | | | | | no |
| MDA- | 4/7 | p.R102L | no | NA | rs73926195:C/ |
| PCa-2B | | | | | T:+:val = YES |
| 22Rv1 | 3/12 | p.W607R | yes | 48576 | dbsnp132- |
| | | | | | no |
| DU- | 527/527 | p.D84N; p.D84H; | yes | 13299 | rs11552822:G/ |
| 145 | | p.D84G; | | | T:−:val = |
| | | p.D84Y; | | | YES |
| | | p.D84D; | | | |
| | | p.D84V | | | |
| DU- | 3/28 | p.T18M | no | NA | dbsnp132- |
| 145 | | | | | no |
| CWR22 | 22/52 | p.A372A | no | NA | dbsnp132- |
| | | | | | no |
| 22Rv1 | 26/57 | p.A372A | no | NA | dbsnp132- |
| | | | | | no |
| LAPC-4 | 13/23 | p.E20K | yes | 96799 | dbsnp132- |
| | | | | | no |
| DU- | 13/102 | p.V22A; p.V22I | no | NA | rs77064436:G/ |
| 145 | | | | | T:+:val = |
| | | | | | YES |
| LNCaP | 7/15 | p.S527S | no | NA | dbsnp132- |
| | | | | | no |
| C4-2B | 9/28 | p.L24L | no | NA | dbsnp132- |
| | | | | | no |
| 22Rv1 | 4/8 | p.A267V | no | NA | dbsnp132- |
| | | | | | no |
| LAPC-4 | 4/10 | p.R944C | no | NA | dbsnp132- |
| | | | | | no |
| DU- | 8/26 | p.H1937P | yes | 70612 | rs75778935:A/ |
| 145 | | | | | C:+:val = |
| | | | | | YES |
| CWR22 | 43/75 | p.T587A | yes | 42946 | dbsnp132- |
| | | | | | no |
| DU- | 5/43 | p.L590L | no | NA | dbsnp 132- |
| 145 | | | | | no |
| C4-2B | 136/541 | p.V118V | no | NA | dbsnp132- |
| | | | | | no |
| LAPC-4 | 10/13 | p.R278L | no | NA | rs117053233:A/ |
| | | | | | G:+:val = |
| | | | | | NO |
| DU- | 6/59 | p.V295L | no | NA | dbsnp132- |
| 145 | | | | | no |
| C4-2B | 21/81 | p.Q209P; | no | NA | dbsnp132- |
| | | p.Q209L | | | no |
| LAPC-4 | 47/85 | p.A114T | yes | 74770 | dbsnp132- |
| | | | | | no |
| LAPC-4 | 20/50 | p.R272H | yes | 74823 | dbsnp132- |
| | | | | | no |
| DU- | 9/64 | p.A3491V | no | NA | dbsnp132- |
| 145 | | | | | no |
| DU- | 9/43 | p.S39L | no | NA | dbsnp132- |
| 145 | | | | | no |
| DU- | 3/30 | p.V14V | no | NA | dbsnp132- |
| 145 | | | | | no |
| LNCaP | 3/5 | p.A548V | no | NA | dbsnp132- |
| | | | | | no |
| LNCaP | 3/27 | p.L32F | no | NA | dbsnp132- |
| | | | | | no |
| DU- | 7/7 | p.R244C | no | NA | dbsnp132- |
| 145 | | | | | no |
| CWR22 | 5/6 | p.A556V | no | NA | dbsnp132- |
| | | | | | no |
| 22Rv1 | 5/7 | p.A556V | no | NA | dbsnp132- |
| | | | | | no |
| LNCaP | 5/9 | p.V780V | no | NA | dbsnp132- |
| | | | | | no |
| CWR22 | 12/22 | p.S253L | yes | 22594 | dbsnp132- |
| | | | | | no |
| 22Rv1 | 21/39 | p.S253L | yes | 22594 | dbsnp132- |
| | | | | | no |
| LAPC-4 | 5/11 | p.R464L | no | NA | dbsnp132- |
| | | | | | no |
| DU- | 4/29 | p.R770Q | yes | 84179 | dbsnp132- |
| 145 | | | | | no |
| LNCaP | 30/55 | p.R786Q | yes | 98942 | rs114347531:C/ |
| | | | | | T:+:val = |
| | | | | | NO |
| C4-2B | 13/18 | p.R786Q | yes | 98942 | rs114347531:C/ |
| | | | | | T:+:val = |
| | | | | | NO |
| MDA- | 9/16 | p.Q319K | yes | 33457 | rs75635495:G/ |
| PCa-2B | | | | | T:+:val = |
| | | | | | YES |
| LAPC-4 | 26/99 | p.A230T | yes | 120973 | dbsnp132- |
| | | | | | no |
| LNCaP | 114/232 | p.G383V | yes | 76092 | dbsnp132- |
| | | | | | no |
| C4-2B | 74/148 | p.G383V | yes | 76092 | dbsnp132- |
| | | | | | no |
| MDA- | 7/8 | p.R130Q; | yes | 5033 | dbsnp132- |
| PCa-2B | | p.R130P; | | | no |
| | | p.R130R; | | | |
| | | p.R130*; | | | |
| | | p.R130L; | | | |
| | | p.R130G | | | |
| LNCaP | 7/18 | p.K922E | no | NA | dbsnp132- |
| | | | | | no |
| LAPC-4 | 4/6 | p.R589H | no | NA | dbsnp132- |
| | | | | | no |
| C4-2B | 3/15 | p.A325A | no | NA | dbsnp132- |
| | | | | | no |
| LAPC-4 | 4/7 | p.R419Q | no | NA | dbsnp132- |
| | | | | | no |
| DU- | 5/5 | p.P179L | no | NA | dbsnp132- |
| 145 | | | | | no |
| LAPC-4 | 7/13 | p.R339W | no | NA | dbsnp132- |
| | | | | | no |
| LNCaP | 14/27 | p.Q399R | yes | 83998 | rs41279894:A/ |
| | | | | | G:+:val = |
| | | | | | YES |
| DU- | 3/22 | p.L78L | no | NA | dbsnp132- |
| 145 | | | | | no |
| LAPC-4 | 64/67 | p.R175L; | yes | 99914 | rs28934578:A/ |
| | | p.R175G; | | | G:−:val = |
| | | p.R175H; | | | NO |
| | | p.R175H | | | |
| DU- | 143/228 | p.V274G; | yes | 10769 | dbsnp132- |
| 145 | | p.V274A; | | | no |
| | | p.V274D; | | | |
| | | p.V274F; | | | |
| | | p.V274G | | | |
| VCaP | 72/72 | p.R248Q; | yes | 120007 | dbsnp132- |
| | | p.R248L; | | | no |
| | | p.R248G; | | | |
| | | p.R248P; | | | |
| | | p.R248W; | | | |
| | | p.R248L; | | | |
| | | p.R248W | | | |
| C4-2B | 20/85 | p.Y234S; | yes | 11152 | dbsnp132- |
| | | p.Y234H; | | | no |
| | | p.Y234C; | | | |
| | | p.Y234N | | | |
| VCaP | 21/144 | p.R448* | no | NA | dbsnp132- |
| | | | | | no |
| C4-2B | 4/17 | p.S308* | no | NA | dbsnp132- |
| | | | | | no |
| LNCaP | 3/14 | p.L1258L | no | NA | dbsnp132- |
| | | | | | no |
| CWR22 | 3/10 | p.A303D | yes | 131334 | rs605088:G/ |
| | | | | | T:+:val = |
| | | | | | YES, rs62332110:G/ |
| | | | | | T:+:val = |
| | | | | | YES |
| LNCaP | 4/6 | p.A303D | yes | 131334 | rs605088:G/ |
| | | | | | T:+:val = |
| | | | | | YES, rs62332110:G/ |
| | | | | | T:+:val = |
| | | | | | YES |
| NCI- | 8/8 | p.A303D | yes | 131334 | rs605088:G/ |
| H660 | | | | | T:+:val = |
| | | | | | YES, rs62332110:G/ |
| | | | | | T:+:val = |
| | | | | | YES |
| C4-2B | 3/3 | p.A303D | yes | 131334 | rs605088:G/ |
| | | | | | T:+:val = |
| | | | | | YES, rs62332110:G/ |
| | | | | | T:+:val = |
| | | | | | YES |

TABLE 9

| | | | | | | | Other | | | | |
|---|---|---|---|---|---|---|---|---|---|---|---|
| | | Samples | | | | CpG | C:G | A:T | | | |
| Gene¹ | Samples | (%) | N | n | PC n | transition | transition | mutation | Indel | P-value | q-value |
| TP53 | 23 | 39.66 | 67,508 | 23 | 4 | 7 | 7 | 5 | 4 | <1.0 × 10⁻⁸ | <1.0 × 10⁻⁶ |
| AR | 5 | 8.62 | 130,852 | 6 | 0 | 0 | 2 | 4 | 0 | 0.00000002 | 0.000194 |
| ZFHX3 | 8 | 13.79 | 627,179 | 9 | 2 | 1 | 1 | 4 | 3 | 0.00000018 | 0.001162 |
| RB1 | 6 | 10.34 | 142,570 | 4 | 1 | 0 | 0 | 0 | 4 | 0.00000025 | 0.001210 |
| PTEN | 6 | 10.34 | 51,493 | 4 | 1 | 0 | 1 | 1 | 2 | 0.00000034 | 0.001317 |
| APC | 6 | 10.34 | 494,165 | 6 | 0 | 0 | 1 | 0 | 5 | 0.00000046 | 0.001485 |
| MLL2 | 5 | 8.62 | 778,982 | 7 | 2 | 2 | 0 | 1 | 4 | 0.00000611 | 0.016903 |
| OR5L1 | 2 | 3.45 | 54,330 | 3 | 0 | 1 | 2 | 0 | 0 | 0.00002369 | 0.057345 |
| CDK12 | 3 | 5.17 | 257,205 | 4 | 0 | 0 | 1 | 0 | 3 | 0.00003993 | 0.085916 |

Territory (N) refers to total covered territory in base pairs across 58 sequenced samples (two of the three distinct METs site samples from the same patient, 43-27 and 43-71, and hyper-mutated sample WA16 were excluded). Total numbers of mutations (n) and numbers of mutations occurring in localized prostate cancer (PC n) are shown for each gene.
¹Genes with more than two somatic mutations occurring in a single sample (KIF16B) have been excluded.

TABLE 10

| CANONICAL | | | | CpG | Other C:G | A:T | |
|---|---|---|---|---|---|---|---|
| PATHWAY¹ | N | n | PC n | transition | transition | mutation | Indel |
| BIOCARTA_P53_PATHWAY | 1,520,550 | 34 | 5 | 7 | 8 | 9 | 10 |
| BIOCARTA_RB_PATHWAY | 1,518,927 | 32 | 5 | 7 | 7 | 8 | 10 |
| SA_G1_AND_S_PHASES | 654,959 | 26 | 4 | 7 | 7 | 7 | 5 |
| BIOCARTA_TID_PATHWAY | 1,545,215 | 32 | 5 | 9 | 9 | 6 | 8 |
| BIOCARTA_RNA_PATHWAY | 912,772 | 24 | 5 | 7 | 8 | 5 | 4 |
| BIOCARTA_G1_PATHWAY | 2,733,258 | 34 | 5 | 7 | 8 | 8 | 11 |
| BIOCARTA_PML_PATHWAY | 1,796,919 | 28 | 4 | 7 | 7 | 5 | 9 |
| BIOCARTA_ARF_PATHWAY | 1,867,081 | 29 | 5 | 7 | 8 | 6 | 8 |
| KEGG_ENDOMETRIAL_CANCER | 5,916,936 | 48 | 5 | 11 | 13 | 12 | 12 |
| KEGG_THYROID_CANCER | 2,649,974 | 35 | 5 | 10 | 11 | 9 | 5 |
| BIOCARTA_CTCF_PATHWAY | 2,257,832 | 31 | 5 | 7 | 9 | 8 | 7 |
| KEGG_BLADDER_CANCER | 3,610,240 | 38 | 4 | 11 | 10 | 9 | 8 |
| BIOCARTA_P53HYPOXIA_PATHWAY | 2,644,050 | 31 | 5 | 7 | 7 | 10 | 7 |
| KEGG_MELANOMA | 5,534,818 | 45 | 6 | 10 | 12 | 13 | 10 |
| BIOCARTA_ATM_PATHWAY | 2,645,938 | 31 | 5 | 7 | 10 | 8 | 6 |
| REACTOME_STABILIZATION_OF_P53 | 3,196,422 | 32 | 5 | 8 | 8 | 8 | 8 |
| BIOCARTA_CHEMICAL_PATHWAY | 2,606,659 | 30 | 6 | 7 | 9 | 8 | 6 |
| KEGG_PROSTATE_CANCER | 9,264,784 | 54 | 8 | 11 | 14 | 17 | 12 |
| KEGG_P53_SIGNALING_PATHWAY | 5,598,653 | 42 | 7 | 8 | 13 | 12 | 9 |
| BIOCARTA_ATRBRCA_PATHWAY | 3,614,559 | 32 | 5 | 7 | 10 | 9 | 6 |
| KEGG_BASAL_CELL_CARCINOMA | 5,608,289 | 42 | 4 | 10 | 10 | 11 | 11 |
| BIOCARTA_TEL_PATHWAY | 2,667,636 | 29 | 4 | 8 | 8 | 5 | 8 |
| KEGG_COLORECTAL_CANCER | 6,151,366 | 41 | 5 | 7 | 14 | 10 | 10 |
| BIOCARTA_G2_PATHWAY | 3,689,873 | 30 | 7 | 7 | 8 | 8 | 7 |
| KEGG_GLIOMA | 6,528,735 | 41 | 6 | 9 | 11 | 11 | 10 |
| KEGG_AMYOTROPHIC_LATERAL_SCLEROSIS_ALS | 4,611,144 | 35 | 7 | 12 | 11 | 7 | 5 |
| KEGG_NON_SMALL_CELL_LUNG_CANCER | 5,280,706 | 35 | 4 | 9 | 9 | 8 | 9 |
| KEGG_PATHWAYS_IN_CANCER | 36,327,764 | 115 | 10 | 24 | 34 | 36 | 21 |
| KEGG_CHRONIC_MYELOID_LEUKEMIA | 6,813,825 | 39 | 5 | 8 | 12 | 10 | 9 |
| KEGG_PANCREATIC_CANCER | 6,825,087 | 38 | 6 | 8 | 13 | 9 | 8 |
| ST_FAS_SIGNALING_PATHWAY | 5,966,096 | 36 | 4 | 8 | 15 | 8 | 5 |
| KEGG_CELL_CYCLE | 12,361,653 | 49 | 8 | 9 | 15 | 11 | 14 |
| KEGG_WNT_SIGNALING_PATHWAY | 13,953,928 | 57 | 7 | 13 | 17 | 14 | 13 |
| KEGG_SMALL_CELL_LUNG_CANCER | 12,315,312 | 51 | 6 | 11 | 16 | 12 | 12 |
| ST_JNK_MAPK_PATHWAY | 4,256,100 | 28 | 6 | 9 | 10 | 5 | 4 |
| REACTOME_CELL_CYCLE_CHECKPOINTS | 8,803,775 | 36 | 5 | 9 | 9 | 9 | 9 |
| KEGG_APOPTOSIS | 7,187,919 | 34 | 7 | 8 | 10 | 10 | 6 |
| REACTOME_BETACATENIN_{—} | 1,555,197 | 12 | 0 | 2 | 2 | 3 | 5 |
| PHOSPHORYLATION_CASCADE | | | | | | | |
| BIOCARTA_P27_PATHWAY | 747,279 | 6 | 0 | 0 | 0 | 1 | 5 |
| ST_ADRENERGIC | 4,487,053 | 18 | 0 | 0 | 4 | 9 | 5 |
| BIOCARTA_PS1_PATHWAY | 2,026,572 | 11 | 0 | 0 | 3 | 2 | 6 |
| REACTOME_APOPTOTIC_EXECUTION_PHASE | 6,878,641 | 30 | 1 | 6 | 12 | 6 | 6 |
| REACTOME_SIGNALING_BY_WNT | 4,229,144 | 17 | 0 | 3 | 3 | 4 | 7 |
| ST_WNT_BETA_CATENIN_PATHWAY | 3,429,466 | 13 | 0 | 0 | 3 | 4 | 6 |
| BIOCARTA_CELLCYCLE_PATHWAY | 1,378,669 | 6 | 0 | 0 | 0 | 1 | 5 |
| BIOCARTA_SKP2E2F_PATHWAY | 745,873 | 5 | 0 | 0 | 0 | 1 | 4 |
| ST_GRANULE_CELL_SURVIVAL_PATHWAY | 2,418,051 | 10 | 0 | 0 | 5 | 0 | 5 |
| BIOCARTA_WNT_PATHWAY | 2,758,479 | 13 | 1 | 1 | 4 | 3 | 5 |
| BIOCARTA_RACCYCD_PATHWAY | 2,000,523 | 8 | 1 | 0 | 1 | 2 | 5 |
| REACTOME_G1_PHASE | 954,684 | 5 | 0 | 0 | 0 | 1 | 4 |
| BIOCARTA_ALK_PATHWAY | 3,466,249 | 15 | 0 | 2 | 4 | 4 | 5 |
| REACTOME_OLFACTORY_SIGNALING_PATHWAY | 17,826,372 | 50 | 4 | 9 | 21 | 16 | 4 |
| BIOCARTA_TGFB_PATHWAY | 2,806,840 | 10 | 1 | 1 | 4 | 0 | 5 |
| BIOCARTA_FAS_PATHWAY | 3,738,640 | 12 | 2 | 1 | 4 | 1 | 6 |
| REACTOME_CYCLIN_E_ASSOCIATED_EVENTS_{—} | 3,506,894 | 9 | 0 | 1 | 1 | 1 | 6 |
| DURING_G1_S_TRANSITION_{—} | | | | | | | |
| BIOCARTA_GSK3_PATHWAY | 2,686,214 | 9 | 0 | 0 | 2 | 2 | 5 |
| REACTOME_ORC1_REMOVAL_FROM_CHROMATIN | 3,979,884 | 10 | 0 | 1 | 3 | 0 | 6 |
| BIOCARTA_PPARA_PATHWAY | 6,993,646 | 8 | 0 | 0 | 0 | 3 | 5 |
| ST_MYOCYTE_AD_PATHWAY | 3,842,014 | 14 | 1 | 3 | 2 | 4 | 5 |
| BIOCARTA_CELL2CELL_PATHWAY | 1,846,306 | 10 | 1 | 3 | 2 | 5 | 0 |
| REACTOME_GENES_INVOLVED_IN_APOPTOTIC_{—} | 6,210,596 | 24 | 0 | 5 | 9 | 5 | 5 |
| CLEAVAGE_OF_CELLULAR_PROTEINS | | | | | | | |
| REACTOME_APOPTOSIS_INDUCED_{—} | 624,910 | 6 | 1 | 1 | 3 | 1 | 1 |
| DNA_FRAGMENTATION | | | | | | | |
| REACTOME_CITRIC_ACID_CYCLE | 1,559,477 | 5 | 1 | 1 | 0 | 0 | 4 |
| REACTOME_GPCR_LIGAND_BINDING | 22,213,171 | 58 | 1 | 31 | 15 | 8 | 4 |
| REACTOME_APOPTOSIS | 11,525,719 | 36 | 1 | 7 | 14 | 7 | 8 |
| KEGG_NEUROACTIVE_LIGAND_{—} | 21,215,515 | 57 | 6 | 27 | 20 | 7 | 3 |
| RECEPTOR_INTERACTION | | | | | | | |
| BIOCARTA_PTEN_PATHWAY | 1,678,489 | 8 | 1 | 1 | 1 | 3 | 3 |
| REACTOME_G_ALPHA_I_SIGNALLING_EVENTS | 10,781,829 | 37 | 0 | 17 | 11 | 6 | 3 |
| BIOCARTA_PITX2_PATHWAY | 3,088,392 | 10 | 0 | 1 | 2 | 2 | 5 |
| KEGG_NEUROTROPHIN_SIGNALING_PATHWAY | 11,279,732 | 39 | 5 | 10 | 16 | 9 | 4 |
| KEGG_OLFACTORY_TRANSDUCTION | 21,286,641 | 55 | 4 | 11 | 23 | 17 | 4 |
| KEGG_TIGHT_JUNCTION | 16,061,382 | 50 | 5 | 15 | 16 | 13 | 6 |
| KEGG_LYSINE_DEGRADATION | 6,178,748 | 18 | 3 | 1 | 8 | 3 | 6 |
| REACTOME_CLASS_A1_RHODOPSIN_{—} | 15,210,497 | 42 | 1 | 22 | 12 | 5 | 3 |
| LIKE_RECEPTORS | | | | | | | |
| BIOCARTA_HCMV_PATHWAY | 1,819,130 | 5 | 0 | 0 | 1 | 0 | 4 |
| BIOCARTA_TNFR1_PATHWAY | 3,465,892 | 8 | 2 | 0 | 2 | 1 | 5 |
| REACTOME_SYNTHESIS_OF_DNA | 6,473,883 | 11 | 1 | 1 | 4 | 0 | 6 |
| REACTOME_E2F_MEDIATED_REGULATION_{—} | 2,082,197 | 4 | 0 | 0 | 0 | 0 | 4 |
| OF_DNA_REPLICATION | | | | | | | |
| REACTOME_PYRUVATE_METABOLISM_{—} | 2,783,649 | 6 | 1 | 2 | 0 | 0 | 4 |
| AND_TCA_CYCLE | | | | | | | |
| KEGG_LONG_TERM_DEPRESSION | 8,518,080 | 25 | 3 | 12 | 6 | 7 | 0 |
| BIOCARTA_HIVNEF_PATHWAY | 6,320,921 | 13 | 2 | 0 | 4 | 3 | 6 |
| SIG_PIP3_SIGNALING_IN_B_LYMPHOCYTES | 4,567,528 | 11 | 1 | 0 | 2 | 7 | 2 |
| REACTOME_PI3K_AKT_SIGNALLING | 3,816,300 | 11 | 1 | 0 | 3 | 5 | 3 |
| REACTOME_SEROTONIN_RECEPTORS | 861,359 | 5 | 0 | 4 | 0 | 1 | 0 |
| REACTOME_METABOLISM_OF_PROTEINS | 13,166,540 | 11 | 1 | 3 | 4 | 0 | 4 |
| BIOCARTA_GRANULOCYTES_PATHWAY | 1,393,333 | 6 | 0 | 3 | 0 | 2 | 1 |
| REACTOME_REGULATION_OF_INSULIN_LIKE_{—} | 1,437,072 | 6 | 0 | 4 | 0 | 2 | 0 |
| GROWTH_FACTOR_ACTIVITY_BY_INSULIN_{—} | | | | | | | |
| LIKE_GROWTH_FACTOR_BINDING_PROTEINS | | | | | | | |
| KEGG_CELL_ADHESION_MOLECULES_CAMS | 12,471,700 | 31 | 5 | 15 | 9 | 7 | 0 |

| | | | Nonsignificant | | | |
|---|---|---|---|---|---|---|
| | | | Gene | | | |
| CANONICAL | P- | | Contribution | APC | TP53 | PTEN |
| PATHWAY¹ | value | q-value | (%) | Counts | Counts | Counts |
| BIOCARTA_P53_PATHWAY | <1.0 × 10⁻⁸ | <1.0 × 10⁻⁶ | 20.59% | 0 | 23 | 0 |
| BIOCARTA_RB_PATHWAY | <1.0 × 10⁻⁸ | <1.0 × 10⁻⁶ | 15.63% | 0 | 23 | 0 |
| SA_G1_AND_S_PHASES | <1.0 × 10⁻⁸ | <1.0 × 10⁻⁶ | 11.54% | 0 | 23 | 0 |
| BIOCARTA_TID_PATHWAY | <1.0 × 10⁻⁸ | <1.0 × 10⁻⁶ | 15.63% | 0 | 23 | 0 |
| BIOCARTA_RNA_PATHWAY | <1.0 × 10⁻⁸ | <1.0 × 10⁻⁶ | 4.17% | 0 | 23 | 0 |
| BIOCARTA_G1_PATHWAY | <1.0 × 10⁻⁸ | <1.0 × 10⁻⁶ | 20.59% | 0 | 23 | 0 |
| BIOCARTA_PML_PATHWAY | <1.0 × 10⁻⁸ | <1.0 × 10⁻⁶ | 3.57% | 0 | 23 | 0 |
| BIOCARTA_ARF_PATHWAY | <1.0 × 10⁻⁸ | <1.0 × 10⁻⁶ | 6.90% | 0 | 23 | 0 |
| KEGG_ENDOMETRIAL_CANCER | <1.0 × 10⁻⁸ | <1.0 × 10⁻⁶ | 31.25% | 6 | 23 | 4 |
| KEGG_THYROID_CANCER | <1.0 × 10⁻⁸ | <1.0 × 10⁻⁶ | 34.29% | 0 | 23 | 0 |
| BIOCARTA_CTCF_PATHWAY | <1.0 × 10⁻⁸ | <1.0 × 10⁻⁶ | 12.90% | 0 | 23 | 4 |
| KEGG_BLADDER_CANCER | <1.0 × 10⁻⁸ | <1.0 × 10⁻⁶ | 28.95% | 0 | 23 | 0 |
| BIOCARTA_P53HYPOXIA_PATHWAY | <1.0 × 10⁻⁸ | <1.0 × 10⁻⁶ | 25.81% | 0 | 23 | 0 |
| KEGG_MELANOMA | <1.0 × 10⁻⁸ | <1.0 × 10⁻⁶ | 31.11% | 0 | 23 | 4 |
| BIOCARTA_ATM_PATHWAY | <1.0 × 10⁻⁸ | <1.0 × 10⁻⁶ | 25.81% | 0 | 23 | 0 |
| REACTOME_STABILIZATION_OF_P53 | <1.0 × 10⁻⁸ | <1.0 × 10⁻⁶ | 28.13% | 0 | 23 | 0 |
| BIOCARTA_CHEMICAL_PATHWAY | <1.0 × 10⁻⁸ | <1.0 × 10⁻⁶ | 23.33% | 0 | 23 | 0 |
| KEGG_PROSTATE_CANCER | <1.0 × 10⁻⁸ | <1.0 × 10⁻⁶ | 31.48% | 0 | 23 | 4 |
| KEGG_P53_SIGNALING_PATHWAY | <1.0 × 10⁻⁸ | <1.0 × 10⁻⁶ | 35.71% | 0 | 23 | 4 |
| BIOCARTA_ATRBRCA_PATHWAY | <1.0 × 10⁻⁸ | <1.0 × 10⁻⁶ | 28.13% | 0 | 23 | 0 |
| KEGG_BASAL_CELL_CARCINOMA | <1.0 × 10⁻⁸ | <1.0 × 10⁻⁶ | 30.95% | 6 | 23 | 0 |
| BIOCARTA_TEL_PATHWAY | <1.0 × 10⁻⁸ | <1.0 × 10⁻⁶ | 6.90% | 0 | 23 | 0 |
| KEGG_COLORECTAL_CANCER | <1.0 × 10⁻⁸ | <1.0 × 10⁻⁶ | 29.27% | 6 | 23 | 0 |
| BIOCARTA_G2_PATHWAY | <1.0 × 10⁻⁸ | <1.0 × 10⁻⁶ | 23.33% | 0 | 23 | 0 |
| KEGG_GLIOMA | <1.0 × 10⁻⁸ | <1.0 × 10⁻⁶ | 24.39% | 0 | 23 | 4 |
| KEGG_AMYOTROPHIC_LATERAL_SCLEROSIS_ALS | <1.0 × 10⁻⁸ | <1.0 × 10⁻⁶ | 34.29% | 0 | 23 | 0 |
| KEGG_NON_SMALL_CELL_LUNG_CANCER | <1.0 × 10⁻⁸ | <1.0 × 10⁻⁶ | 22.86% | 0 | 23 | 0 |
| KEGG_PATHWAYS_IN_CANCER | <1.0 × 10⁻⁸ | <1.0 × 10⁻⁶ | 62.61% | 6 | 23 | 4 |
| KEGG_CHRONIC_MYELOID_LEUKEMIA | <1.0 × 10⁻⁸ | <1.0 × 10⁻⁶ | 30.77% | 0 | 23 | 0 |
| KEGG_PANCREATIC_CANCER | <1.0 × 10⁻⁸ | <1.0 × 10⁻⁶ | 28.95% | 0 | 23 | 0 |
| ST_FAS_SIGNALING_PATHWAY | 0.00000001 | <1.0 × 10⁻⁶ | 36.11% | 0 | 23 | 0 |
| KEGG_CELL_CYCLE | 0.00000001 | <1.0 × 10⁻⁶ | 44.90% | 0 | 23 | 0 |
| KEGG_WNT_SIGNALING_PATHWAY | 0.00000002 | 0.000001 | 49.12% | 6 | 23 | 0 |
| KEGG_SMALL_CELL_LUNG_CANCER | 0.00000002 | 0.000001 | 39.22% | 0 | 23 | 4 |
| ST_JNK_MAPK_PATHWAY | 0.00000003 | 0.000001 | 17.86% | 0 | 23 | 0 |
| REACTOME_CELL_CYCLE_CHECKPOINTS | 0.00000052 | 0.000013 | 36.11% | 0 | 23 | 0 |
| KEGG_APOPTOSIS | 0.00000104 | 0.000025 | 32.35% | 0 | 23 | 0 |
| REACTOME_BETACATENIN_{—} | 0.00000144 | 0.000033 | 50.00% | 6 | 0 | 0 |
| PHOSPHORYLATION_CASCADE | | | | | | |
| BIOCARTA_P27_PATHWAY | 0.00000362 | 0.000082 | 33.33% | 0 | 0 | 0 |
| ST_ADRENERGIC | 0.0000052 | 0.000114 | 33.33% | 6 | 0 | 0 |
| BIOCARTA_PS1_PATHWAY | 0.00000603 | 0.000129 | 45.45% | 6 | 0 | 0 |
| REACTOME_APOPTOTIC_EXECUTION_PHASE | 0.0000387 | 0.000811 | 80.00% | 6 | 0 | 0 |
| REACTOME_SIGNALING_BY_WNT | 0.0000412 | 0.000843 | 64.71% | 6 | 0 | 0 |
| ST_WNT_BETA_CATENIN_PATHWAY | 0.00004466 | 0.000893 | 53.85% | 6 | 0 | 0 |
| BIOCARTA_CELLCYCLE_PATHWAY | 0.00007417 | 0.00145 | 33.33% | 0 | 0 | 0 |
| BIOCARTA_SKP2E2F_PATHWAY | 0.00008437 | 0.001614 | 20.00% | 0 | 0 | 0 |
| ST_GRANULE_CELL_SURVIVAL_PATHWAY | 0.00010024 | 0.001877 | 40.00% | 6 | 0 | 0 |
| BIOCARTA_WNT_PATHWAY | 0.00018699 | 0.003428 | 53.85% | 6 | 0 | 0 |
| BIOCARTA_RACCYCD_PATHWAY | 0.00022647 | 0.004042 | 50.00% | 0 | 0 | 0 |
| REACTOME_G1_PHASE | 0.00022965 | 0.004042 | 20.00% | 0 | 0 | 0 |
| BIOCARTA_ALK_PATHWAY | 0.0004257 | 0.007345 | 60.00% | 6 | 0 | 0 |
| REACTOME_OLFACTORY_SIGNALING_PATHWAY | 0.00043744 | 0.007403 | 94.00% | 0 | 0 | 0 |
| BIOCARTA_TGFB_PATHWAY | 0.00068035 | 0.011296 | 40.00% | 6 | 0 | 0 |
| BIOCARTA_FAS_PATHWAY | 0.00074873 | 0.012172 | 66.67% | 0 | 0 | 0 |
| REACTOME_CYCLIN_E_ASSOCIATED_EVENTS_{—} | 0.00076072 | 0.012172 | 55.56% | 0 | 0 | 0 |
| DURING_G1_S_TRANSITION_{—} | | | | | | |
| BIOCARTA_GSK3_PATHWAY | 0.0008376 | 0.013162 | 33.33% | 6 | 0 | 0 |
| REACTOME_ORC1_REMOVAL_FROM_CHROMATIN | 0.00099666 | 0.015387 | 60.00% | 0 | 0 | 0 |
| BIOCARTA_PPARA_PATHWAY | 0.00114601 | 0.017388 | 50.00% | 0 | 0 | 0 |
| ST_MYOCYTE_AD_PATHWAY | 0.00121402 | 0.01786 | 57.14% | 6 | 0 | 0 |
| BIOCARTA_CELL2CELL_PATHWAY | 0.00121772 | 0.01786 | 100.00% | 0 | 0 | 0 |
| REACTOME_GENES_INVOLVED_IN_APOPTOTIC_{—} | 0.00131054 | 0.018702 | 75.00% | 6 | 0 | 0 |
| CLEAVAGE_OF_CELLULAR_PROTEINS | | | | | | |
| REACTOME_APOPTOSIS_INDUCED_{—} | 0.00131763 | 0.018702 | 100.00% | 0 | 0 | 0 |
| DNA_FRAGMENTATION | | | | | | |
| REACTOME_CITRIC_ACID_CYCLE | 0.00154239 | 0.021297 | 100.00% | 0 | 0 | 0 |
| REACTOME_GPCR_LIGAND_BINDING | 0.00154889 | 0.021297 | 100.00% | 0 | 0 | 0 |
| REACTOME_APOPTOSIS | 0.00179142 | 0.024253 | 83.33% | 6 | 0 | 0 |
| KEGG_NEUROACTIVE_LIGAND_{—} | 0.00185044 | 0.024673 | 100.00% | 0 | 0 | 0 |
| RECEPTOR_INTERACTION | | | | | | |
| BIOCARTA_PTEN_PATHWAY | 0.00188792 | 0.024797 | 50.00% | 0 | 0 | 4 |
| REACTOME_G_ALPHA_I_SIGNALLING_EVENTS | 0.00198824 | 0.02573 | 100.00% | 0 | 0 | 0 |
| BIOCARTA_PITX2_PATHWAY | 0.00237569 | 0.029714 | 40.00% | 6 | 0 | 0 |
| KEGG_NEUROTROPHIN_SIGNALING_PATHWAY | 0.00238884 | 0.029714 | 41.03% | 0 | 23 | 0 |
| KEGG_OLFACTORY_TRANSDUCTION | 0.00239735 | 0.029714 | 94.55% | 0 | 0 | 0 |
| KEGG_TIGHT_JUNCTION | 0.00254144 | 0.031062 | 92.00% | 0 | 0 | 4 |
| KEGG_LYSINE_DEGRADATION | 0.0026602 | 0.032068 | 100.00% | 0 | 0 | 0 |
| REACTOME_CLASS_A1_RHODOPSIN_{—} | 0.00322987 | 0.038409 | 100.00% | 0 | 0 | 0 |
| LIKE_RECEPTORS | | | | | | |
| BIOCARTA_HCMV_PATHWAY | 0.00407302 | 0.047535 | 20.00% | 0 | 0 | 0 |
| BIOCARTA_TNFR1_PATHWAY | 0.00410527 | 0.047535 | 50.00% | 0 | 0 | 0 |
| REACTOME_SYNTHESIS_OF_DNA | 0.00417445 | 0.047708 | 63.64% | 0 | 0 | 0 |
| REACTOME_E2F_MEDIATED_REGULATION_{—} | 0.00426308 | 0.048096 | 0.00% | 0 | 0 | 0 |
| OF_DNA_REPLICATION | | | | | | |
| REACTOME_PYRUVATE_METABOLISM_{—} | 0.00437348 | 0.048717 | 100.00% | 0 | 0 | 0 |
| AND_TCA_CYCLE | | | | | | |
| KEGG_LONG_TERM_DEPRESSION | 0.00488266 | 0.05335 | 100.00% | 0 | 0 | 0 |
| BIOCARTA_HIVNEF_PATHWAY | 0.00491059 | 0.05335 | 69.23% | 0 | 0 | 0 |
| SIG_PIP3_SIGNALING_IN_B_LYMPHOCYTES | 0.00626687 | 0.067254 | 63.64% | 0 | 0 | 4 |
| REACTOME_PI3K_AKT_SIGNALLING | 0.00692897 | 0.073464 | 63.64% | 0 | 0 | 4 |
| REACTOME_SEROTONIN_RECEPTORS | 0.00709024 | 0.074279 | 100.00% | 0 | 0 | 0 |
| REACTOME_METABOLISM_OF_PROTEINS | 0.00763364 | 0.079031 | 100.00% | 0 | 0 | 0 |
| BIOCARTA_GRANULOCYTES_PATHWAY | 0.00778185 | 0.079628 | 100.00% | 0 | 0 | 0 |
| REACTOME_REGULATION_OF_INSULIN_LIKE_{—} | 0.00854438 | 0.086426 | 100.00% | 0 | 0 | 0 |
| GROWTH_FACTOR_ACTIVITY_BY_INSULIN_{—} | | | | | | |
| LIKE_GROWTH_FACTOR_BINDING_PROTEINS | | | | | | |
| KEGG_CELL_ADHESION_MOLECULES_CAMS | 0.00882807 | 0.088281 | 100.00% | 0 | 0 | 0 |

| CANONICAL | AR | RB1 | ZFHX3 | MLL2 | OR5L1 | CDK12 |
|---|---|---|---|---|---|---|
| PATHWAY¹ | Counts | Counts | Counts | Counts | Counts | Counts |
| BIOCARTA_P53_PATHWAY | 0 | 4 | 0 | 0 | 0 | 0 |
| BIOCARTA_RB_PATHWAY | 0 | 4 | 0 | 0 | 0 | 0 |
| SA_G1_AND_S_PHASES | 0 | 0 | 0 | 0 | 0 | 0 |
| BIOCARTA_TID_PATHWAY | 0 | 4 | 0 | 0 | 0 | 0 |
| BIOCARTA_RNA_PATHWAY | 0 | 0 | 0 | 0 | 0 | 0 |
| BIOCARTA_G1_PATHWAY | 0 | 4 | 0 | 0 | 0 | 0 |
| BIOCARTA_PML_PATHWAY | 0 | 4 | 0 | 0 | 0 | 0 |
| BIOCARTA_ARF_PATHWAY | 0 | 4 | 0 | 0 | 0 | 0 |
| KEGG_ENDOMETRIAL_CANCER | 0 | 0 | 0 | 0 | 0 | 0 |
| KEGG_THYROID_CANCER | 0 | 0 | 0 | 0 | 0 | 0 |
| BIOCARTA_CTCF_PATHWAY | 0 | 0 | 0 | 0 | 0 | 0 |
| KEGG_BLADDER_CANCER | 0 | 4 | 0 | 0 | 0 | 0 |
| BIOCARTA_P53HYPOXIA_PATHWAY | 0 | 0 | 0 | 0 | 0 | 0 |
| KEGG_MELANOMA | 0 | 4 | 0 | 0 | 0 | 0 |
| BIOCARTA_ATM_PATHWAY | 0 | 0 | 0 | 0 | 0 | 0 |
| REACTOME_STABILIZATION_OF_P53 | 0 | 0 | 0 | 0 | 0 | 0 |
| BIOCARTA_CHEMICAL_PATHWAY | 0 | 0 | 0 | 0 | 0 | 0 |
| KEGG_PROSTATE_CANCER | 6 | 4 | 0 | 0 | 0 | 0 |
| KEGG_P53_SIGNALING_PATHWAY | 0 | 0 | 0 | 0 | 0 | 0 |
| BIOCARTA_ATRBRCA_PATHWAY | 0 | 0 | 0 | 0 | 0 | 0 |
| KEGG_BASAL_CELL_CARCINOMA | 0 | 0 | 0 | 0 | 0 | 0 |
| BIOCARTA_TEL_PATHWAY | 0 | 4 | 0 | 0 | 0 | 0 |
| KEGG_COLORECTAL_CANCER | 0 | 0 | 0 | 0 | 0 | 0 |
| BIOCARTA_G2_PATHWAY | 0 | 0 | 0 | 0 | 0 | 0 |
| KEGG_GLIOMA | 0 | 4 | 0 | 0 | 0 | 0 |
| KEGG_AMYOTROPHIC_LATERAL_SCLEROSIS_ALS | 0 | 0 | 0 | 0 | 0 | 0 |
| KEGG_NON_SMALL_CELL_LUNG_CANCER | 0 | 4 | 0 | 0 | 0 | 0 |
| KEGG_PATHWAYS_IN_CANCER | 6 | 4 | 0 | 0 | 0 | 0 |
| KEGG_CHRONIC_MYELOID_LEUKEMIA | 0 | 4 | 0 | 0 | 0 | 0 |
| KEGG_PANCREATIC_CANCER | 0 | 4 | 0 | 0 | 0 | 0 |
| ST_FAS_SIGNALING_PATHWAY | 0 | 0 | 0 | 0 | 0 | 0 |
| KEGG_CELL_CYCLE | 0 | 4 | 0 | 0 | 0 | 0 |
| KEGG_WNT_SIGNALING_PATHWAY | 0 | 0 | 0 | 0 | 0 | 0 |
| KEGG_SMALL_CELL_LUNG_CANCER | 0 | 4 | 0 | 0 | 0 | 0 |
| ST_JNK_MAPK_PATHWAY | 0 | 0 | 0 | 0 | 0 | 0 |
| REACTOME_CELL_CYCLE_CHECKPOINTS | 0 | 0 | 0 | 0 | 0 | 0 |
| KEGG_APOPTOSIS | 0 | 0 | 0 | 0 | 0 | 0 |
| REACTOME_BETACATENIN_{—} | 0 | 0 | 0 | 0 | 0 | 0 |
| PHOSPHORYLATION_CASCADE | | | | | | |
| BIOCARTA_P27_PATHWAY | 0 | 4 | 0 | 0 | 0 | 0 |
| ST_ADRENERGIC | 6 | 0 | 0 | 0 | 0 | 0 |
| BIOCARTA_PS1_PATHWAY | 0 | 0 | 0 | 0 | 0 | 0 |
| REACTOME_APOPTOTIC_EXECUTION_PHASE | 0 | 0 | 0 | 0 | 0 | 0 |
| REACTOME_SIGNALING_BY_WNT | 0 | 0 | 0 | 0 | 0 | 0 |
| ST_WNT_BETA_CATENIN_PATHWAY | 0 | 0 | 0 | 0 | 0 | 0 |
| BIOCARTA_CELLCYCLE_PATHWAY | 0 | 4 | 0 | 0 | 0 | 0 |
| BIOCARTA_SKP2E2F_PATHWAY | 0 | 4 | 0 | 0 | 0 | 0 |
| ST_GRANULE_CELL_SURVIVAL_PATHWAY | 0 | 0 | 0 | 0 | 0 | 0 |
| BIOCARTA_WNT_PATHWAY | 0 | 0 | 0 | 0 | 0 | 0 |
| BIOCARTA_RACCYCD_PATHWAY | 0 | 4 | 0 | 0 | 0 | 0 |
| REACTOME_G1_PHASE | 0 | 4 | 0 | 0 | 0 | 0 |
| BIOCARTA_ALK_PATHWAY | 0 | 0 | 0 | 0 | 0 | 0 |
| REACTOME_OLFACTORY_SIGNALING_PATHWAY | 0 | 0 | 0 | 0 | 3 | 0 |
| BIOCARTA_TGFB_PATHWAY | 0 | 0 | 0 | 0 | 0 | 0 |
| BIOCARTA_FAS_PATHWAY | 0 | 4 | 0 | 0 | 0 | 0 |
| REACTOME_CYCLIN_E_ASSOCIATED_EVENTS_{—} | 0 | 4 | 0 | 0 | 0 | 0 |
| DURING_G1_S_TRANSITION_{—} | | | | | | |
| BIOCARTA_GSK3_PATHWAY | 0 | 0 | 0 | 0 | 0 | 0 |
| REACTOME_ORC1_REMOVAL_FROM_CHROMATIN | 0 | 4 | 0 | 0 | 0 | 0 |
| BIOCARTA_PPARA_PATHWAY | 0 | 4 | 0 | 0 | 0 | 0 |
| ST_MYOCYTE_AD_PATHWAY | 0 | 0 | 0 | 0 | 0 | 0 |
| BIOCARTA_CELL2CELL_PATHWAY | 0 | 0 | 0 | 0 | 0 | 0 |
| REACTOME_GENES_INVOLVED_IN_APOPTOTIC_{—} | 0 | 0 | 0 | 0 | 0 | 0 |
| CLEAVAGE_OF_CELLULAR_PROTEINS | | | | | | |
| REACTOME_APOPTOSIS_INDUCED_{—} | 0 | 0 | 0 | 0 | 0 | 0 |
| DNA_FRAGMENTATION | | | | | | |
| REACTOME_CITRIC_ACID_CYCLE | 0 | 0 | 0 | 0 | 0 | 0 |
| REACTOME_GPCR_LIGAND_BINDING | 0 | 0 | 0 | 0 | 0 | 0 |
| REACTOME_APOPTOSIS | 0 | 0 | 0 | 0 | 0 | 0 |
| KEGG_NEUROACTIVE_LIGAND_{—} | 0 | 0 | 0 | 0 | 0 | 0 |
| RECEPTOR_INTERACTION | | | | | | |
| BIOCARTA_PTEN_PATHWAY | 0 | 0 | 0 | 0 | 0 | 0 |
| REACTOME_G_ALPHA_I_SIGNALLING_EVENTS | 0 | 0 | 0 | 0 | 0 | 0 |
| BIOCARTA_PITX2_PATHWAY | 0 | 0 | 0 | 0 | 0 | 0 |
| KEGG_NEUROTROPHIN_SIGNALING_PATHWAY | 0 | 0 | 0 | 0 | 0 | 0 |
| KEGG_OLFACTORY_TRANSDUCTION | 0 | 0 | 0 | 0 | 3 | 0 |
| KEGG_TIGHT_JUNCTION | 0 | 0 | 0 | 0 | 0 | 0 |
| KEGG_LYSINE_DEGRADATION | 0 | 0 | 0 | 0 | 0 | 0 |
| REACTOME_CLASS_A1_RHODOPSIN_{—} | 0 | 0 | 0 | 0 | 0 | 0 |
| LIKE_RECEPTORS | | | | | | |
| BIOCARTA_HCMV_PATHWAY | 0 | 4 | 0 | 0 | 0 | 0 |
| BIOCARTA_TNFR1_PATHWAY | 0 | 4 | 0 | 0 | 0 | 0 |
| REACTOME_SYNTHESIS_OF_DNA | 0 | 4 | 0 | 0 | 0 | 0 |
| REACTOME_E2F_MEDIATED_REGULATION_{—} | 0 | 4 | 0 | 0 | 0 | 0 |
| OF_DNA_REPLICATION | | | | | | |
| REACTOME_PYRUVATE_METABOLISM_{—} | 0 | 0 | 0 | 0 | 0 | 0 |
| AND_TCA_CYCLE | | | | | | |
| KEGG_LONG_TERM_DEPRESSION | 0 | 0 | 0 | 0 | 0 | 0 |
| BIOCARTA_HIVNEF_PATHWAY | 0 | 4 | 0 | 0 | 0 | 0 |
| SIG_PIP3_SIGNALING_IN_B_LYMPHOCYTES | 0 | 0 | 0 | 0 | 0 | 0 |
| REACTOME_PI3K_AKT_SIGNALLING | 0 | 0 | 0 | 0 | 0 | 0 |
| REACTOME_SEROTONIN_RECEPTORS | 0 | 0 | 0 | 0 | 0 | 0 |
| REACTOME_METABOLISM_OF_PROTEINS | 0 | 0 | 0 | 0 | 0 | 0 |
| BIOCARTA_GRANULOCYTES_PATHWAY | 0 | 0 | 0 | 0 | 0 | 0 |
| REACTOME_REGULATION_OF_INSULIN_LIKE_{—} | 0 | 0 | 0 | 0 | 0 | 0 |
| GROWTH_FACTOR_ACTIVITY_BY_INSULIN_{—} | | | | | | |
| LIKE_GROWTH_FACTOR_BINDING_PROTEINS | | | | | | |
| KEGG_CELL_ADHESION_MOLECULES_CAMS | 0 | 0 | 0 | 0 | 0 | 0 |

TABLE 11

| | KEGG enrichments | PINdb enrichments |
|---|---|---|
| Genes | (p-value, corrected) | (p-value, corrected) |
| CYP2C9 CYP1A1 | C21-Steroid hormone | |
| CYP11A1 CYB5R3 | metabolism (4e−7) | |
| CYB5R1 SCD POR | Linoleic acid metabolism | |
| CYB5A CYCS | (0.01) | |
| CYP17A1 NDOR1 | Retinol metabolism | |
| CYP2C19 CYP2E1 | (5e−3) | |
| CYP11B2 CYP11B1 | Metabolism of xenobiotics | |
| FDX1 UQCRC2 | by cytochrome P450 | |
| CYC1 UQCRC1 | (2e−4) | |
| | Drug metabolism- | |
| | cytochrome P450 | |
| | (0.02) | |
| | Parkinson's disease | |
| | (0.03) | |
| GRP MC3R NPFFR2 | Neuroactive ligand- | |
| AGRP PMCH NPY2R | receptor interaction (0.01) | |
| NPY MC4R MC5R | | |
| MEP1B | | |
| EFNB3 EFNB1 RPL8 | Axon guidance (<1e−13) | |
| EPHA8 EPHA7 | | |
| EPHA5 EPHA4 | | |
| EPHA3 EPHA2 | | |
| ARL15 SORBS1 | | |
| TIAM1 EPHB3 | | |
| ESRRG EPHB1 | | |
| ARHGEF15 EFNA5 | | |
| EFNA4 EFNA3 LAT | | |
| SLA PTPN13 | | |
| C11orf49 GRIP1 | | |
| CIR1 POLR2C | Purine metabolism (6e−3) | PolII(G); RNAPII; |
| C19orf2 CTDP1 | Pyrimidine metabolism | Gdown1-containing Pol |
| REXO1 TAF11 | (6e−4) | II (1e−8) |
| NDRG2 CPSF1 | RNA polymerase (7e−7) | TAP-tagged RNAPII |
| GTF2E1 SET KLF5 | Basal transcription factors | RNAPII; RNA |
| EAF2 SF3A2 TAF6 | (<1e−13) | polymerase II (7e−11) |
| TCEA1 IWS1 | Huntington's disease | TAF4b-TFIID TAF4b- |
| POLR2D HTATSF1 | (3e−4) | TFIID; 4b-IID; 4b/4- |
| POLR2B POLR2H | | IID (<1e−13) |
| POLR2K TAF7 | | TAF4-TFIID TAF4- |
| POLR2E TAF5 TAF4 | | TFIID; 4-IID; 4/4-IIB |
| TAF2 TAF1 GTF2F2 | | (<1e−13) |
| TAF9 TAF8 | | MLL1-WDR5 MLL1- |
| SUPT4H1 UBE2W | | WDR5 (4e−4) |
| | | TFIID hTFIID; |
| | | transcription initiation |
| | | factor (7e−13) |
| | | DAB TFIID-IIA-IIB |
| | | transcription initiation |
| | | (7e−13) |
| | | RNA polymerase II |
| | | DNA-directed RNA |
| | | polymerase II; RNAP |
| | | II; RNAPII; RNA pol II; |
| | | RNA polI (1e−8) |
| | | TFTC SAGA-like; |
| | | hSAGA; TBP-free |
| | | TAFII-containing (6e−8) |
| | | Integrator DSS1- |
| | | associated; RNAPII- |
| | | associated (6e−4) |
| FCN2 C4B MASP1 | Complement and | |
| MASP2 LEPR LEP | coagulation cascades | |
| C9 C8B C2 MMP25 | (3e−8) | |
| CPN2 CPN1 CLU C5 | Systemic lupus | |
| | erythematosus (4e−4) | |
| EXOSC10 DOM3Z | RNA degradation (<1e−13) | |
| DIS3 ZBTB17 | | |
| RPS20 MPZL1 | | |
| SKIV2L2 GTF2IRD1 | | |
| MPP6 UPF3B | | |
| EXOSC8 SKIV2L | | |
| EXOSC9 EXOSC5 | | |
| MPHOSPH6 SMPD4 | | |
| NUP160 EXOSC4 | | |
| EXOSC1 | | |
| GORASP2 UBE2D2 | Circadian rhythm- | |
| RIBC2 RAB2A | mammal (2e−12) | |
| DYRK2 FAM71C | | |
| RAD54B BLZF1 | | |
| TIMELESS CRY1 | | |
| CRY2 CSNK1D | | |
| PSMA1 DMC1 PJA1 | | |
| PJA2 ABCD4 | | |
| CCDC33 PER3 PER2 | | |
| PER1 TMEM66 | | |
| MAGED1 CSNK1E | | |
| AKAP4 PPP1CC | Apoptosis (4e−4) | |
| AKAP3 PRKACB | Insulin signaling pathway | |
| PKIA ROPN1L | (5e−5) | |
| PRKAR2A NBEA | | |
| PRKAR2B PRKAR1A | | |
| AKAP11 PRKX | | |
| FSIP2 FSIP1 | | |
| NLRP1 TRAF5 CD40 | NOD-like receptor | |
| BCL10 TLR2 RIPK2 | signaling pathway (1e−5) | |
| NOD1 NOD2 LY96 | | |
| NSMAF CARD6 | | |
| MALT1 | | |
| SELL NCF2 NCF4 | Cell adhesion molecules | |
| CD46 SELPLG | (CAMs) (0.02) | |
| CD93 CYBB MSN | Leukocyte transendothelial | |
| ICAM2 SPN | migration (0.02) | |
| KLF12 EHMT2 | | CtBP; CtBP corepressor; |
| ARNT ZFPM2 SGTB | | CtBP |
| GATA1 JARID2 | | corepressor; CtBP1- |
| RAI2 TBX5 CTBP1 | | containing (0.01) |
| CTBP2 HEY1 GATA4 | | |
| FBXW7 | | |
| ZNF639 TSPYL2 | | A-Med Mediator |
| MED13L CNTROB | | (9e−4) |
| MED12 MED13 | | TR-TRAP TR/TRAP; |
| DGCR14 CDK8 | | TRAP; TRAP/SMCC; |
| APTX LYST | | Mediator (2e−3) |
| | | mMediator mammalian |
| | | Mediator; Mediator |
| | | (1e−4) |
| | | TRAP/SMCC Mediator- |
| | | T/S; TRAP; SMCC; |
| | | thyroid hormone |
| | | receptor associated |
| | | protein; Srb/Med- |
| | | containing; mediator |
| | | (7e−3) |
| SAP30 RBP1 | | Sin3-CII Sin3 complex |
| ADIPOR1 RBBP7 | | II; Sin3-p33ING1b- |
| RBP2 APPL1 APPL2 | | containing; Sin3- |
| MBD3 DHX30 LRAT | | p33ING1b; ING1b- |
| | | Sin3-containing; Sin3- |
| | | p33ING1b-containing; |
| | | Sin3-HDAC (5e−4) |
| | | Sin3-CI Sin3CI; Sin3 |
| | | complex I; p33ING1- |
| | | Sin3; ING1b-Sin3- |
| | | containing; Sin3- |
| | | p33ING1b-containing |
| | | (2e−3) |
| | | 1ALL-1 (0.02) |
| TNKS KARS KTN1 | | TIN2/TRF1 TRF1; |
| ACD ATM POT1 ATR | | TRF1/TIN2; telosome |
| MCL1 FANCD2 | | (2e−3) |
| TMEM11 HPRT1 | | TRF1-TIN2 TRF1.TIN2; |
| TREX1 CBFA2T2 | | TIN2; telosome; |
| TERF1 RPIA NBN | | shelterin (0.01) |
| DOK5 TNKS2 | | |
| CHEK2 HEXDC | | |
| PCBD1 BNIP3L | | |
| TPT1 MDC1 | | |
| CELSR2 RINT1 | | |
| SALL1 RAB40C | | |
| GARS BNIP3 | | |
| FHOD1 NME1 NME2 | | |
| GLRA2 EEF1D PSD | | |
| MLC1 RAD50 | | |
| LNPEP DCTPP1 | | |
| STEAP3 WDYHV1 | | |
| SYTL5 SYTL4 | | |
| RAB27B RPH3AL | | |
| RAPGEF4 UNC13D | | |
| RAB3A CACNA1S | | |
| RIMS2 MLPH | | |
| OSTM1 RGS20 | | |
| STMN2 CLCN7 MX1 | | |
| TEX11 SIX2 NAGK | | |
| DACH1 RGS6 | | |
| MAPK10 GNAZ | | |
| TRPC4 TRPC5 | | |
| ITPR2 SIX3 EYA1 | | |
| GNAI3 | | |
| NMT LPXN RPL18A | | |
| MRPL28 SLC4A4 | | |
| GLUL SLC4A1 | | |
| PRPSAP1 EDNRB | | |
| CHIC2 PRPS2 MBIP | | |
| PLEKHF2 NONO | | |
| HTR2A ZNF263 | | |
| EDN1 SPEF1 NOS3 | | |
| CETP PSPC1 LPL | | |
| PTCHD2 ANKS1B | | |
| PTPN4 ZNRD1 | | |
| ANKZF1 NME3 CA2 | | |
| GRK6 UBE2Z | | |
| GNA13 HOXA1 | | |
| GNA11 | | |
| FOXP3 PABPC1 | | |
| C3orf63 PAIP2B | | |
| EP400 NAV1 | | |
| C12orf35 PAN3 | | |
| PAN2 TBC1D4 | | |
| ETF1 CIB1 PGR | | |
| HDAC8 HNRNPD | | |
| GSPT2 NFAT5 | | |
| RUNX1T1 TRRAP | | |
| SYCP3 UBR5 | | |
| SYNCRIP GFI1 | | |
| TOPBP1 EIF4G3 | | |
| PLAG1 EIF4G1 | | |
| PAIP2 KPNA2 | | |
| KPNA1 TAF9B | | |
| ZMIZ1 EIF4A1 | | |
| ZNF652 | | |
| CHGB STK11 | | |
| UBE2L6 PI4K2B | | |
| UBE2L3 S100A13 | | |
| PHYHIP SPATC1 | | |
| MAGI3 MAGI2 | | |
| CASR MAST1 | | |
| MAST3 SUFU | | |
| RNF19A BAI1 ANG | | |
| TUBG1 PTEN ZZEF1 | | |
| BAIAP3 HDAC11 | | |
| PPIE | | |
| OBSCN MYH2 | | |
| ANKRD1 ANKRD2 | | |
| NEB RING1 DGKH | | |
| FHL1 MYBPC3 TTN | | |
| MYBPC1 CAPN3 | | |
| CBX4 DYSF DGKD | | |
| HUS1B NHP2L1 | | |
| RAD17 FAM124A | | |
| HUS1 KIAA1712 | | |
| ZFHX3 SPERT | | |
| ZNF250 PIAS3 | | |
| NKAP RBM10 | | |
| ZNF165 RAD9B | | |
| MYB NINL | | |
| ZNF638 WWP1 | | |
| CLCN5 IL6R IL6 | | |
| EGFL6 KLF2 CPSF6 | | |
| GIN1 SH3GL2 | | |
| ACTA2 MINK1 | | |
| PCYT1B NEFM NEFL | | |
| NEFH CCT5 | | |
| TMSB4X GC MYL1 | | |
| DMD SNTB1 | | |
| MAPK12 DGKZ | | |
| TLL1 TWSG1 SAG | | |
| CXCR1 SGCZ | | |
| SCN4A PPP1R9B | | |
| KCNJ12 SNTG1 | | |
| CHRM2 ABR DTNA | | |
| CADPS ADRA1A | | |
| ADRA1B ADRA1D | | |
| BMP1 CADPS | | |
| CHRD SCN5A | | |

TABLE 12

| | | | C4- | | DU- | |
|---|---|---|---|---|---|---|
| | Average | 22Rv1 | 2B | CWR22 | 145 | LAPC-4 |
| Reads | 54,927,162 | 23,730,575 | 25,519,818 | 27,765,968 | 175,811,526 | 29,571,652 |
| sequenced | | | | | | |
| (after quality | | | | | | |
| filtering) | | | | | | |
| Bases | 2,066,490,044 | 922,714,325 | 1,020,792,720 | 1,110,638,720 | 6,680,837,988 | 1,182,866,080 |
| sequenced | | | | | | |
| (after quality | | | | | | |
| filtering) | | | | | | |
| Bases | 1,340,937,482 | 616,855,460 | 772,176,520 | 873,950,240 | 4,585,832,932 | 900,847,920 |
| mapped to | | | | | | |
| genome¹ | | | | | | |
| Genes | 12,376 | 11,826 | 10,944 | 12,248 | 14,042 | 11,855 |
| expressed (of | | | | | | |
| 19,365)² | | | | | | |
| Known SNPs | 5,892 | 4,773 | 4,904 | 5,537 | 7,967 | 5,758 |
| (Coding³, | | | | | | |
| Point) | | | | | | |
| Known SNPs | 2,278 | 1,809 | 1,835 | 2,106 | 3,317 | 2,241 |
| (Non-syn, | | | | | | |
| Point) | | | | | | |
| Known SNPs | 13 | 6 | 13 | 9 | 51 | 15 |
| (Coding, | | | | | | |
| Indel⁴) | | | | | | |
| Novel | 1,111 | 944 | 1,787 | 863 | 2,184 | 2,179 |
| variants⁵ | | | | | | |
| (Coding, | | | | | | |
| Point) | | | | | | |
| Novel | 756 | 648 | 1,245 | 586 | 1,556 | 1,482 |
| variants | | | | | | |
| (Non-syn, | | | | | | |
| Point) | | | | | | |
| Novel | 275 | 128 | 170 | 150 | 1,745 | 197 |
| variants | | | | | | |
| (Coding, | | | | | | |
| Indel) | | | | | | |

| | | MDA- | | | | |
|---|---|---|---|---|---|---|
| | | PCa- | NCI- | | | WPE1- |
| | LNCaP⁶ | 2B | H660 | PC3 | VCaP⁶ | NB26 |
| Reads | 100,609,731 | 26,682,646 | 24,645,212 | 25,898,842 | 122,774,968 | 21,187,840 |
| sequenced | | | | | | |
| (after quality | | | | | | |
| filtering) | | | | | | |
| Bases | 3,480,001,429 | 1,067,305,840 | 985,808,480 | 1,009,133,430 | 4,423,777,870 | 847,513,600 |
| sequenced | | | | | | |
| (after quality | | | | | | |
| filtering) | | | | | | |
| Bases | 2,073,018,786 | 837,105,080 | 793,900,160 | 702,244,540 | 1,933,581,099 | 660,799,560 |
| mapped to | | | | | | |
| genome¹ | | | | | | |
| Genes | 13,064 | 12,040 | 12,936 | 12,054 | 13,841 | 11,289 |
| expressed (of | | | | | | |
| 19,365)² | | | | | | |
| Known SNPs | 7,395 | 6,736 | 5,600 | 4,940 | 6,853 | 4,354 |
| (Coding³, | | | | | | |
| Point) | | | | | | |
| Known SNPs | 2,958 | 2,479 | 2,129 | 1,884 | 2,724 | 1,571 |
| (Non-syn, | | | | | | |
| Point) | | | | | | |
| Known SNPs | 7 | 12 | 5 | 8 | 18 | 4 |
| (Coding, | | | | | | |
| Indel⁴) | | | | | | |
| Novel | 1,983 | 1,061 | 327 | 205 | 427 | 258 |
| variants⁵ | | | | | | |
| (Coding, | | | | | | |
| Point) | | | | | | |
| Novel | 1,399 | 666 | 189 | 125 | 262 | 163 |
| variants | | | | | | |
| (Non-syn, | | | | | | |
| Point) | | | | | | |
| Novel | 85 | 127 | 61 | 123 | 148 | 95 |
| variants | | | | | | |
| (Coding, | | | | | | |
| Indel) | | | | | | |

¹Number of reads mapped back to the human reference genome sequence hg18 (NCBI 36.1, March 2006) plus Illumina's Refseq derived splice junctions trimmed to ensure at least two bases overlapping the splice junction.
²Total number of human CCDS genes with >=1 read of average coverage.
³Coding region defined by set of CCDS transcripts.
⁴Present in >=3 reads after removal of PCR duplicates.
⁵Present in >=3 reads after removal of PCR duplicates and present in 20% of the coverage.
⁶LNCaP and VCaP transcriptome sequence from pooled sequences from an untreated sample and four androgen time course samples for each cell line.

TABLE 13

| | Name | |
|---|---|---|
| Use | (direction) | Sequence |
| Sequencing | FOXA1-F1 | GTAAAACGACGGCCAGTCCTCCAGTGCCCACCACTAACC |
| Sequencing | FOXA1-R1 | GTGCGGGTAGCTGCGCTTGAA |
| Sequencing | FOXA1-F2 | GTAAAACGACGGCCAGTATGGCGTACGCGCCGTCCAA |
| Sequencing | FOXA1-R2 | AGGGGTCCTTGCGGCTCTCA |
| Sequencing | FOXA1-F3 | GTAAAACGACGGCCAGTAGCGCTTCAAGTGCGAGAAGC |
| Sequencing | FOXA1-R3 | CCCTTTCAGGTGCAGCTGGGA |
| Sequencing | FOXA1-F4 | GTAAAACGACGGCCAGTCGGAGTTGAAGACTCCAGCCTCCTC |
| Sequencing | FOXA1-R4 | ACCAGCATGGCTATGCCAGACAA |
| qPCR | GAPDH-F | TGCACCACCAACTGCTTAGC |
| qPCR | GAPDH-R | GGCATGGACTGTGGTCATGAG |
| qPCR | ACTB-F | AGGATGCAGAAGGAGATCACTG |
| qPCR | ACTB-R | AGTACTTGCGCTCAGGAGGAG |
| qPCR | MLL-F | CGCCAAGCTCTTTGCTAAAGGAAAC |
| qPCR | MLL-R | TTCTCACATTTGGAATGGACCCAGC |
| qPCR | ASH2L-F | AACCACTTTGCAGTGCCAGACTGG |
| qPCR | ASH2L-R | GACCAAGTTTGCCTCCCTGGGT |
| qPCR | PSA-F | ACGCTGGACAGGGGGCAAAAG |
| qPCR | PSA-R | GGGCAGGGCACATGGTTCACT |
| qPCR | FOXA1-F | GAAGACTCCAGCCTCCTCAACTG |
| qPCR | FOXA1-R | TGCCTTGAAGTCCAGCTTATGC |
| qPCR | DLX1-F | GCGGCCTCTTTGGGACTCACAC |
| qPCR | DLX1-R | GGCCAACGCACTACCCTCCAGA |

TABLE 14

| | Estimated | | | | | |
|---|---|---|---|---|---|---|
| | tumor | Modi- | >1 copy | 1 copy | 1 copy | >1 copy |
| Sample | content | fied?¹ | loss | loss | gain | gain |
| T8 | 69% | No | −0.675 | −0.321 | 0.321 | 0.675 |
| T12 | 69% | No | −0.677 | −0.322 | 0.322 | 0.677 |
| T32 | 35% | No | −0.383 | −0.175 | 0.175 | 0.383 |
| T90 | 56% | Yes | −0.566 | −0.265 | 0.23 | 0.566 |
| T91 | 77% | No | −0.733 | −0.351 | 0.351 | 0.733 |
| T92 | 53% | No | −0.543 | −0.253 | 0.253 | 0.543 |
| T93 | 47% | Yes | −0.494 | −0.215 | 0.175 | 0.494 |
| T94 | 31% | Yes | −0.339 | −0.17 | 0.17 | 0.47 |
| T95 | 39% | Yes | −0.418 | −0.13 | 0.192 | 0.418 |
| T96 | 74% | No | −0.713 | −0.341 | 0.341 | 0.713 |
| T97 | 72% | Yes | −0.68 | −0.331 | 0.331 | 0.695 |
| WA3 | 73% | Yes | −0.703 | −0.3 | 0.335 | 0.703 |
| WA7 | 74% | Yes | −0.75 | −0.34 | 0.34 | 0.712 |
| WA10 | 82% | No | −0.768 | −0.37 | 0.37 | 0.768 |
| WA11 | 58% | Yes | −0.584 | −0.274 | 0.34 | 0.584 |
| WA12 | 79% | Yes | −1.2 | −0.6 | 0.36 | 0.85 |
| WA13 | 75% | Yes | −1.35 | −0.6 | 0.7 | 1.35 |
| WA14 | 42% | Yes | −0.85 | −0.451 | 0.451 | 0.72 |
| WA15 | 76% | No | −0.726 | −0.348 | 0.348 | 0.726 |
| WA16 | 81% | No | −0.767 | −0.369 | 0.369 | 0.767 |
| WA17 | 69% | Yes | −0.5 | −0.25 | 0.321 | 0.676 |
| WA18 | 84% | No | −0.783 | −0.378 | 0.378 | 0.783 |
| WA19 | 53% | No | −0.544 | −0.254 | 0.254 | 0.544 |
| WA20 | 63% | Yes | −0.6 | −0.297 | 0.297 | 0.6 |
| WA22 | 72% | No | −0.694 | −0.331 | 0.331 | 0.694 |
| WA23 | 79% | Yes | −0.78 | −0.358 | 0.3 | 0.6 |
| WA24 | 100% | Yes | −0.95 | −0.439 | 0.439 | 0.896 |
| WA25 | 70% | No | −0.683 | −0.325 | 0.325 | 0.683 |
| WA26 | 58% | Yes | −0.65 | −0.276 | 0.276 | 0.595 |
| WA27 | 44% | Yes | −0.51 | −0.216 | 0.216 | 0.469 |
| WA28 | 77% | Yes | −0.8 | −0.4 | 0.351 | 0.733 |
| WA29 | 40% | Yes | −0.426 | −0.13 | 0.15 | 0.426 |
| WA30 | 60% | Yes | −0.75 | −0.282 | 0.282 | 1 |
| WA31 | 79% | Yes | −0.748 | −0.359 | 0.25 | 0.748 |
| WA32 | 82% | Yes | −1.2 | −0.4 | 0.371 | 0.771 |
| WA33 | 54% | Yes | −0.75 | −0.5 | 0.35 | 0.8 |
| WA35 | 63% | Yes | −0.625 | −0.2 | 0.4 | 0.8 |
| WA37 | 56% | No | −0.572 | −0.268 | 0.268 | 0.572 |
| WA38 | 58% | No | −0.585 | −0.274 | 0.274 | 0.585 |
| WA39 | 64% | No | −0.637 | −0.301 | 0.301 | 0.637 |
| WA40 | 92% | Yes | −1.4 | −0.75 | 0.5 | 1.25 |
| WA41 | 61% | No | −0.614 | −0.289 | 0.289 | 0.614 |
| WA42 | 74% | Yes | −0.9 | −0.34 | 0.34 | 0.712 |
| WA43-27 | 82% | No | −0.773 | −0.372 | 0.372 | 0.773 |
| WA43-44 | 73% | No | −0.704 | −0.336 | 0.336 | 0.704 |
| WA43-71 | 70% | Yes | −1.2 | −0.682 | 0.682 | 1.2 |
| WA46 | 79% | Yes | −0.9 | −0.359 | 0.5 | 1 |
| WA47 | 75% | Yes | −0.717 | −0.343 | 0.2 | 0.717 |
| WA48 | 54% | No | −0.555 | −0.259 | 0.259 | 0.555 |
| WA49 | 74% | No | −0.716 | −0.342 | 0.342 | 0.716 |
| WA50 | 44% | Yes | −0.6 | −0.216 | 0.216 | 0.7 |
| WA51 | 75% | No | −0.722 | −0.345 | 0.345 | 0.722 |
| WA52 | 64% | Yes | −0.633 | −0.299 | 0.2 | 0.633 |
| WA53 | 78% | Yes | −1.2 | −0.5 | 0.5 | 0.746 |
| WA54 | 67% | No | −0.66 | −0.313 | 0.313 | 0.66 |
| WA55 | 74% | Yes | −1 | −0.5 | 0.5 | 1.1 |
| WA56 | 76% | Yes | −0.725 | −0.347 | 0.36 | 1 |
| WA57 | 56% | Yes | −0.574 | −0.269 | 0.269 | 0.75 |
| WA58 | 56% | No | −0.568 | −0.266 | 0.266 | 0.568 |
| WA59 | 75% | No | −0.718 | −0.343 | 0.343 | 0.718 |
| WA60 | 61% | Yes | −1.1 | −0.7 | 0.6 | 1 |

### Example 2

### Focal Deletion of SPOPL in Prostate Cancer

Genes recurrently mutated, subject to copy number gain or loss, or involved in chromosomal rearrangements often play driving roles in cancer development and can serve as the basis for molecular subtyping. In prostate cancer, robust molecular subtypes have been identified, based largely on the presence or absence of gene fusions involving the 5′ regions of androgen regulated genes and ETS transcription factor family members, most commonly TMPRSS2:ERG (Beltran et al., Clin Cancer Res 19, 517 (Feb. 1, 2013); Rubin et al., J Clin Oncol 29, 3659 (Sep. 20, 2011); Tomlins et al., Eur Urol 56, 275 (Apr. 24, 2009); Tomlins et al., Science 310, 644 (Oct. 28, 2005)). Specific alteration identified in prostate cancers without ETS fusions include SPINK1 over-expression (Tomlins et al., Cancer Cell 13, 519 (June, 2008)), loss or mutation of CHD1 (Grasso et al., Nature 487, 239 (Jul. 12, 2012); Huang et al., Oncogene 31, 4164 (Sep. 13, 2012); Liu et al., Oncogene 31, 3939 (Aug. 30, 2012); Barbieri et al., Nat Genet. 44, 685 (June, 2012); Berger et al., Nature 470, 214 (Feb. 10, 2011)), and mutations in SPOP (Grasso et al., supra; Barbieri et al., supra; Berger et al., supra; Kan et al., Diverse somatic mutation patterns and pathway alterations in human cancers. Nature 466, 869 (Aug. 12, 2010)), which encodes the substrate-binding subunit of a Cullin-based E3 ubiquitin ligase (Kan et al., Diverse somatic mutation patterns and pathway alterations in human cancers. Nature 466, 869 (Aug. 12, 2010)). Although these alterations are only found in ETS fusion negative cancers, they can co-occur. CHD1 deletions have been identified as occurring exclusively in ETS fusion negative cancers (Grasso et al., supra). Experiments described herein resulted in the identification of recurrent, focal homozygous deletions in SPOPL, the homologue of SPOP, in ˜1% of prostate cancers, which are ETS fusion negative. By aCGH, T56, an ETS fusion negative localized prostate cancer, showed a high level copy loss of CHD1 (chr 5q), as well as a high level copy loss of SPOPL (chr 2q). By FISH, homozygous deletion of SPOPL in T56 was confirmed. Together, the results demonstrate that loss of SPOPL is a recurrent alteration in ETS fusion negative prostate cancers.

### Methods:

Genome wide copy number profiles from prostate cancers from 4 studies (The Cancer Genome Atlas [TCGA] and (Grasso et al., supra; Demichelis et al., Genes Chromosomes Cancer 48, 366 (April, 2009); Taylor et al., Cancer Cell 18, 11 (Jul. 13, 2010)) were visualized using the Oncomine Powertools DNA Copy Number Browser (Grasso et al., supra).

### FISH:

Fluorescence in situ hybridization was performed essentially as described (Bhalla et al.,. Mod Pathol, (Jan. 25, 2013)). Two BAC probes overlying SPOPL (RP11-243M18 and RP11-656A4) were fluorescently labeled using nick translation and confirmed to bind to 2q22.1 by hybridization to normal human lymphocyte metaphase spreads. 4 uM sections were cut from formalin fixed paraffin embedded tissue from T56, a localized prostate cancer previously subjected to aCGH(6). FISH using RP11-243M18 and a chromosome 2 centromeric probe (Abbot Molecular Labs, CEP 2 (D2Z1)) was performed on two separate slides containing the index cancer focus from T56. FISH scoring for SPOPL was performed manually under 100× oil immersion objective in non-overlapping and morphologically intact nuclei. More than 50 cells were scored from the cancer tissue. Areas of cancer tissue with weak or no signals and benign adjacent areas were not included in the analysis. For SPOPL, normal signal pattern was recorded by the presence of separate red (two) and green (two) signals for chromosome 2 centromeric control and SPOPL locus probes, respectively. Homozygous deletion was considered present if both copies of SPOPL locus probes were lost in the presence of >2 signals for chromosome 2 control probe in >30% of cells. This cutoff was determined based on the evaluation of normal prostate glands and stroma.

### Results

FIG. 22 shows that copy number profiling identifies focal deletion of SPOPL in prostate cancer. A. Genome wide copy number profiles from 545 prostate cancers from 4 studies were visualized using the Oncomine Powertools DNA Copy Number Browser. The sum of the log 2 copy number for each segmented sample is plotted in genomic order. The location of known genes harboring recurrent copy number gains/losses or mutations are indicated. A novel peak of copy number loss was identified at chromosome 2q22.1. B. High resolution view of chromosome 2 from A. The top panel shows the peak of copy number loss at 2q22.1. The expanded view shows individual samples as rows, with indicated genes represented by boxes. The size of each box indicates the binned copy number call (log 2, according to the legend key). Only samples with at least one gene in the region with a log 2 copy number<−1.0 are shown, and missing boxes indicate that gene has no called log 2 copy number<1.0. C. Genome wide copy number plot for T56, which harbors a focal, homozygous deletion on 2q22.1 including SPOPL, as well as a focal high level deletion on 5q21 including CHD1.

FIG. 23 shows that fluorescence in situ hybridization (FISH) confirms homozygous deletion of SPOPL in T56. A. FISH probes were generated from BAC clones overlying SPOPL on 2q22.1 (RP11-243M18; RP11-656A4). Correct localization was confirmed by hybridization to normal human lymphocyte metaphase spreads, showing single singles at chromosome 2q22.1. B. Probes for SPOPL (RP11-243M18) and chromosome 2 centromeric region (Abbot Molecular) were applied to formalin fixed paraffin embedded tissue sections from T56, a localized prostate cancer with homozygous SPOPL deletion by aCGH (see FIG. 22). The left panel shows stromal cells (bottom) with equal SPOPL and chromosome 2 centromeric signals, while cancerous cells (top) show complete loss of SPOPL signals, consistent with homozygous deletion. Similar findings in a separate field of cancerous cells is shown in the right panel.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of screening for the presence of metastatic castrate resistant prostate cancer (CRPC) in a sample from a subject, comprising
(a) contacting a biological sample from a subject with a reagent that specifically detects a mutation or level of expression in one or more genes selected from the group consisting of: v-ets erythroblastosis virus E26 oncogene homolog 2 (avian) (ETS2), Myeloid/lymphoid or mixed-lineage leukemia (MLL), Myeloid/lymphoid or mixed-lineage leukemia 3 (MLL3), Myeloid/lymphoid or mixed-lineage leukemia 5 (MLL5), Myeloid/lymphoid or mixed-lineage leukemia 2 (MLL2), Forkhead box A1 (FOXA1), Lysine (K)-specific demethylase 6A (UTX), and Additional sex combs like 2 (Drosophila) (ASXL1); and
(b) detecting the presence of a mutation in one more genes selected from the group consisting of the level of expression of ETS2, MLL, MLL3, MLL5, MLL2, FOXA1, UTX, and ASXL1 using an in vitro assay,
wherein the presence of said mutation is indicative of CRCP in said subject.

2. The method of claim 1, wherein the sample is selected from the group consisting of tissue, blood, plasma, serum, urine, urine supernatant, urine cell pellet, semen, prostatic secretions and prostate cells.

3. The method of claim 1, wherein detection is carried out utilizing a method selected from the group consisting of a sequencing technique, a nucleic acid hybridization technique, a nucleic acid amplification technique, and an immunoassay.

4. The method of claim 3, wherein the nucleic acid amplification technique is selected from the group consisting of polymerase chain reaction, reverse transcription polymerase chain reaction, transcription-mediated amplification, ligase chain reaction, strand displacement amplification, and nucleic acid sequence based amplification.

5. The method of claim 1, wherein said reagent is selected from the group consisting of a pair of amplification oligonucleotides and an oligonucleotide probe.

6. The method of claim 1, wherein said mutation is a loss of function mutation.

7. The method of claim 6, wherein said ETS2 mutation is R437c.

8. The method of claim 6, wherein said MLL mutation is Q1815fp.

9. The method of claim 6, wherein said MLL3 mutation is selected from the group consisting of R1742fs and F4463fs.

10. The method of claim 6, wherein said MLL5 mutation is E1397fs.

11. The method of claim 6, wherein said ASXL2 mutation is selected from the group consisting of Y1163*, Q1104*, Q172*, P749fs, L2240V and R2248*.

12. The method of claim 6, wherein said FOXA1 mutation is selected from the group consisting of S453fs and F400I.

13. A method of screening for the presence of metastatic castrate resistant prostate cancer (CRPC) in a sample from a subject, comprising
(a) contacting a biological sample from a subject with a reagent that specifically detects a deletion of ETS2; and
(b) detecting the presence of a deletion of ETS2 using an in vitro assay,
wherein the presence of said deletion is indicative of CRCP in said subject.

14. The method of claim 13, wherein the sample is selected from the group consisting of tissue, blood, plasma, serum, urine, urine supernatant, urine cell pellet, semen, prostatic secretions and prostate cells.

15. The method of claim 13, wherein detection is carried out utilizing a method selected from the group consisting of a sequencing technique, a nucleic acid hybridization technique, a nucleic acid amplification technique, and an immunoassay.

16. The method of claim 15, wherein the nucleic acid amplification technique is selected from the group consisting of polymerase chain reaction, reverse transcription polymerase chain reaction, transcription-mediated amplification, ligase chain reaction, strand displacement amplification, and nucleic acid sequence based amplification.

17. The method of claim 13, wherein said reagent is selected from the group consisting of a pair of amplification oligonucleotides and an oligonucleotide probe.

18. A method of screening for the presence of prostate cancer in a sample from a subject, comprising
(a) contacting a biological sample from a subject with a reagent that specifically detects a deletion of SPOPL; and
(b) detecting the presence of a deletion of SPOPL using an in vitro assay,
wherein the presence of said deletion is indicative of prostate cancer in said subject.

19. The method of claim 18, wherein said prostate cancer is an ETS fusion negative prostate cancer.

20. The method of claim 17, wherein said reagent is selected from the group consisting of a pair of amplification oligonucleotides and an oligonucleotide probe.
